SEQUENCE LISTING (1 sequence)

SEQ ID NO: 1 - DNA, 30 bp, synthetic construct
  source 1..30: mol_type = other DNA
ttttttttttggctccactaaatagacgca

(19) United States
(12) Patent Application Publication
(10) Pub. No.: US 2024/0218437 A1
(43) Pub. Date: Jul. 4, 2024

(54) METHODS AND COMPOSITIONS FOR ASSESSING PERFORMANCE

(71) Applicant: 10x Genomics, Inc., Pleasanton, CA (US)

(72) Inventors: Zahra Kamila BELHOCINE, Pleasanton, CA (US); Zachary W. BENT, Pleasanton, CA (US); Rajiv BHARADWAJ, Pleasanton, CA (US); Alexander GAGNON, Pleasant Hill, CA (US); Qiang GONG, Pleasanton, CA (US); Ashley HAYES, Oakland, CA (US); Benjamin HINDSON, Pleasanton, CA (US); Yuwei LI, Belmont, CA (US); Benjamin PRUITT, Cambridge, MA (US); Daniel P. RIORDAN, Hillsborough, NC (US); Hiroshi SASAKI, Dublin, CA (US); Weiyi TANG, Dublin, CA (US)

(21) Appl. No.: 18/542,038

(22) Filed: Dec. 15, 2023

Related U.S. Application Data
(60) Provisional application No. 63/433,386, filed on Dec. 16, 2022.

(51) Int. Cl. C12Q 1/6844; C12Q 1/6816

(52) U.S. Cl. CPC C12Q 1/6846; C12Q 1/6816

(57) ABSTRACT

The present disclosure relates in some aspects to methods and compositions for assessing system performance for in situ analyte detection. In some aspects, performance of an individual instrument can be assessed, or the performance of two or more instruments can be assessed and optionally compared. In some aspects, disclosed herein is a method comprising using rolling circle amplification products (RCPs) deposited on a cell-free and tissue-free quality control (QC) slide to assess performance of instrument workflow, where an instrument is used to decode signals associated with the RCPs on the QC slide. Quality metrics associated with the decoding (e.g., a percentage of RCPs successfully decoded to genes) can be used to qualify a system comprising the instrument and reagents for in situ analyte detection in cells or tissue samples, e.g., using in situ probe hybridization or in situ sequencing performed on the instrument.

### CROSS-REFERENCE TO RELATED APPLICATION

This application claims priority to U.S. Provisional Application No. 63/433,386, filed Dec. 16, 2022, entitled “Methods and Compositions for Assessing Performance,” which is herein incorporated by reference in its entirety for all purposes.

### REFERENCE TO AN ELECTRONIC SEQUENCE LISTING

The contents of the electronic sequence listing (202412018300SEQLIST.xml; Size: 1,948 bytes; and Date of Creation: Dec. 15, 2023) is herein incorporated by reference in its entirety.

### FIELD

The present disclosure relates in some aspects to methods, compositions, kits, and systems for assessing performance of an instrument and assay components (e.g., reagents in/on the instrument) for analyzing biological molecules in situ in a sample.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. For example, in situ assays are important tools for understanding the molecular basis of cell identity and developing treatment for diseases. Improved instruments, systems, and workflows for performing in situ assays are needed. The present disclosure addresses such and other needs.

### SUMMARY

Assays detecting analytes in situ (e.g., using in situ probe hybridization or in situ sequencing) can involve complex equipment, reagents, and procedures, and may be prone to instrument error. In some aspects, provided herein is a performance test for assessing a workflow performed using an instrument for in situ analyte detection, including methods, assays, workflows, reagents, kits, and systems for qualifying the instrument workflow for use in in situ analysis of a cell or tissue sample.

In some aspects, the instrument performance test provided herein facilitates assessment and/or comparison of the performance of one or more instruments. In some aspects, the assessment is quantitative and/or qualitative. In some aspects, the instrument performance test is performed in vitro on a test slide with known nucleic acid molecules to allow a user to confidently assess whether the instrument, system, and assay components (e.g., reagents provided on-instrument) performs as expected, identify errors relating to instrument functions (e.g., functions of a fluidics module, reagents, an optics module, and a system controller for reagent delivery, imaging, and decoding), and/or assess instrument-to-instrument variations. In some aspects, the performance test utilizes a standardized qualification slide having nucleic acid molecules (e.g., rolling circle amplification products, RCPs) produced using a standardized protocol and independent of a cell or tissue sample. In some embodiments, the methods and compositions provided herein are used to troubleshoot the performance of an instrument (e.g., as described in Section III).

In some aspects, the RCPs collectively comprise a standardized set of identifier sequences (e.g., barcode sequences) that are decodable using a predetermined set of reagents (e.g., intermediate probes and fluorescently labeled probes) that are delivered using the instrument to the qualification slide in sequential cycles. For instance, different sets of reagents are contacted with the qualification slide in a predetermined order in order to decode the identifier sequences (e.g., barcode sequences) in the RCPs. In some aspects, the performance test is carried without using any cell or tissue sample. In some embodiments, the performance of an instrument in decoding RCPs on the standardized qualification slide is assessed, and one or more performance metrics relating to decoding the RCPs to signal code sequences in a codebook are used to qualify the instrument. In some aspects, the performance test produces expected results if instrument performance is satisfactory. In some aspects, if instrument performance is not optimal and/or is unsatisfactory, the performance test produces results that fail one or more performance metrics. In some embodiments, an instrument that passes a performance test disclosed herein is used for analyte detection in situ in a cell or tissue sample, e.g., for in situ analysis of RNA (e.g., a subset of a transcriptome), cDNA, genomic DNA, and/or non-nucleic acid analytes.

In some aspects, provided herein is a kit comprising: a rolling circle amplification product (RCP); and a solid support comprising a functional group for immobilizing the RCP on the solid support. In some aspects, the kit further comprises a particle.

In some aspects, provided herein is a kit comprising: a plurality of rolling circle amplification products (RCPs); and a solid support comprising functional groups for immobilizing the plurality of RCPs on the solid support. In some aspects, the kit further comprises a plurality of particles. In some embodiments, the solid support comprising functional groups for immobilizing the plurality of RCPs and/or the plurality of particles on the solid support. In some embodiments, the plurality of RCPs are immobilized on the solid support, and/or the plurality of particles are immobilized on the solid support. In some embodiments, the plurality of RCPs are directly immobilized on the solid support. In some embodiments, the plurality of RCPs are directly immobilized on the solid support via the functional groups on the solid support. In some embodiments, the plurality of RCPs are directly or indirectly immobilized on the plurality of particles which are deposited on the solid support. In some embodiments, the plurality of particles are directly or indirectly immobilized on the solid support.

In some embodiments, the plurality of particles comprise beads (e.g., hydrogel beads or latex beads) coupled to detectable labels. In some embodiments, the size and/or shape of an individual bead is comparable to that of a nucleus in a cell or tissue sample. In some embodiments, the diameters of the beads is between about 0.5 μm and about 20 μm. In some embodiments, the mean diameter of the beads is between about 0.5 μm and about 3 μm. In some embodiments, the mean diameter of the beads is between about 3 μm and about 16 μm, such as about 10 μm. In some embodiments, the diameters of the beads is between about 5 μm and about 15 μm. In some embodiments, the mean diameter of the beads is about 2 μm. In some embodiments, a particle of the plurality of particles can have a size and/or shape mimicking that of a cell, e.g., a cell in a tissue section of analysis using a microscope. In some embodiments, the plurality of particles comprises round beads and/or oval beads (e.g., beads coupled to fluorescent moieties such as a blue fluorescent dye). In some embodiments, the signal intensity of detectable labels on an individual bead is comparable to the signal intensity of a nucleus detected in a cell or tissue sample.

In some embodiments, the detectable labels in the particles have an excitation wavelength between about 300 nm and about 400 nm. In some embodiments, the detectable labels have a maximum excitation wavelength between about 325 and 375 nm. In some embodiments, the detectable labels have a maximum excitation wavelength of about 350 nm. In some embodiments, the detectable labels have a maximum excitation wavelength of about 360 nm. In some embodiments, the detectable labels have an emission wavelength between about 400 nm and about 600 nm. In some embodiments, the detectable labels have a maximum emission wavelength of about 460 nm. In some embodiments, the detectable labels in the particles are non-autofluorescent. In some embodiments, the detectable labels in the particles are substantially nonfluorescent under excitation wavelengths of about 490 nm, about 530 nm, about 590 nm, and/or about 650 nm. In some embodiments, the detectable labels in the particles comprise a fluorescent dye. In some embodiments, the detectable labels in the particles are or comprise a blue fluorescent dye. In some embodiments, the particles (e.g., beads) are functionalized to attach to the solid support. In some embodiments, the particles (e.g., beads) comprising tethering moieties. In some embodiments, the detectable labels comprise a blue fluorescent dye. In some embodiments, the beads are modified with tethering moieties. In some embodiments, the beads are amine-modified and/or comprise a biotin or a derivative or analog thereof. In some embodiments, the beads are hydrogel beads or latex beads. In some embodiments, the beads comprise hydrogel beads. In some embodiments, the beads are amine-modified polystyrene beads. In some embodiments, the ratio between the number of the particles and the number of RCPs is about 1:5 or lower, about 1:10 or lower, about 1:1,00 or lower, about 1:500 or lower, about 1:1,000 or lower, or about 1:5,000 or lower.

In some embodiments, any one or more of the RCPs are generated outside a cell or tissue sample. In some embodiments, any one or more of the RCPs are generated outside a cell or tissue sample, generated in a solution, and/or generated in vivo. In some embodiments, the RCPs are not in a cell or tissue. In some embodiments, the RCPs are in solution, dried, or lyophilized. In some embodiments, the RCPs are in solution. In some embodiments, the RCPs are dried. In some embodiments, the RCPs are lyophilized. In some embodiments, the kit comprises one or more vials and each vial comprises RCPs in solution, dried RCPs, or lyophilized RCPS. In some embodiments, the kit comprises one or more vials and each vial comprises RCPs in solution. In some embodiments, the kit comprises one or more vials and each vial comprises dried RCPs. In some embodiments, the kit comprises one or more vials and each vial comprises lyophilized RCPs. In some embodiments, the RCPs are generated on an additional particle. In some embodiments, the additional particle is functionalized. For example, the additional particle is functionalized with an acrydite moiety. In some embodiments, the additional particle is a bead (e.g., a gel bead). In some cases, the additional particle comprises a plurality of oligonucleotides and each of the plurality of oligonucleotides comprises a primer sequence complementary to a sequence of the circularized probe. In some embodiments, the RCPs are generated on a functionalized additional particle using the oligonucleotide as primer and a circular or circularized probe as template. In some embodiments, the RCPs generated on the additional particle are deposited on the solid support. In some embodiments, the additional particle has a diameter between about 1 μm and about 50 μm. In some embodiments, the additional particle has a diameter of about 10 μm.

In some embodiments, any one or more of the RCPs are generated in solution. In some embodiments, any one or more of the RCPs are generated in vitro. In some embodiments, the RCPs are not in a cell or tissue. In some embodiments, any one or more of the RCPs are in solution. In some embodiments, any one or more of the RCPs are dried. In some embodiments, any one or more of the RCPs are lyophilized. In some embodiments, any one or more of the RCPs are dried. In some embodiments, any one or more of the RCPs are freeze-dried. In some embodiments, the kit comprises one or more vials, each vial comprising RCPs in solution, dried RCPs, or lyophilized RCPs. In some embodiments, the RCPs in different vials are pooled. For instance, contents of a vial containing RCPs in solution are combined with another vial containing RCPs in solution. In other instances, contents of a vial containing lyophilized RCPs are combined with another vial containing lyophilized RCPs. In some embodiments, RCPs in solution can also be pooled with lyophilized RCPs. In other instances, contents of a vial containing dried RCPs are combined with contents of another vial containing dried RCPs.

In some embodiments, each RCP of the plurality of RCPs comprises multiple copies of an identifier sequence. In some embodiments, each RCP (as well as the identifier sequence therein) is associated with an assigned signal code sequence from a codebook. In some embodiments, each different identifier sequence is assigned a different signal code sequence from a codebook. In some embodiments, each different identifier sequence and the RCP comprising it is uniquely identified by a signal code sequence from a codebook. In some embodiments, the identifier sequence are associated with a gene or a product of the gene, and the identifier sequence comprises or is a barcode sequence that corresponds to the gene or product thereof.

In some embodiments, the kit comprises detectably labeled probes configured to hybridize to the identifier sequences in the RCPs. In some embodiments, the kit comprises intermediate probes configured to hybridize to the identifier sequences in the RCPs, and detectably labeled probes configured to hybridize to at least some of the intermediate probes. In some embodiments, each intermediate probe comprises i) a recognition sequence configured to hybridize to one of the identifier sequences, and ii) a hybridization sequence configured to hybridize to one of the detectably labeled probes. In some embodiments, the hybridization sequence is in a 3′ overhang of an intermediate probe. In some embodiments, the hybridization sequence is in a 5′ overhang of an intermediate probe. In some embodiments, the hybridization sequence is in a 3′ overhang and a 5′ overhang of an intermediate probe (e.g., the hybridization sequence is in a split region of the intermediate probe). In some embodiments, the detectably labeled probes are detectable under excitation wavelengths of about 490 nm, about 530 nm, about 590 nm, or about 650 nm. In some embodiments, one or more of the identifier sequences is a sequence of a gene or complement thereof. In some embodiments, one or more of the identifier sequences comprises a barcode sequence, e.g., one that corresponds to a sequence of a gene or complement thereof. In some embodiments, the barcode sequence is an artificial sequence that corresponds to a sequence of a gene or complement thereof. In some embodiments, the number of different identifier sequences in the RCPs is at least 4, at least 6, at least 8, at least 10, at least 12, at least 14, at least 16, or more. In some embodiments, the number of different detectably labeled probes is 3, 4, 5, 6, 7, or 8. In some embodiments, the number of different detectably labeled probes is 4, and the number of different identifier sequences in the RCPs is 9 or more. In some embodiments, each different identifier sequence corresponds to a different reference gene. In some embodiments, a plurality of reference genes and identifier sequences associated therewith are used for qualifying an instrument. In some embodiments, the number of different detectably labeled probes in the kit is at most or about 3, at most or about 4, at most or about 5, at most or about 6, at most or about 7, or at most or about 8. In some embodiments, each different detectably labeled probe has a different nucleic acid sequence (e.g., one that hybridizes to the hybridization sequence of an intermediate probe disclosed herein) and a corresponding different detectable label, e.g., a fluorophore of a different color under a fluorescent microscope. In some embodiments, the number of different detectably labeled probes is 4 and the number of different identifier sequences in the RCPs is 9 or more.

In some embodiments, the solid support comprises a planar substrate. In some embodiments, the solid support comprises a glass or plastic substrate. In some embodiments, the plurality of RCPs and/or the plurality of particles are immobilized in two, three, four, or more discrete regions on the solid support. In some embodiments, one or more of the discrete regions each comprises one or more fiducial markers. In some embodiments, any two or more of the discrete regions are non-overlapping.

In some embodiments, the plurality of RCPs and/or the plurality of particles comprises a functional group configured to react with the functional groups of the solid support, e.g., to form covalent bonds. In some embodiments, the functional group of the plurality of RCPs and/or the plurality of particles comprises amine and the functional groups of the solid support comprise an N-hydroxysuccinimide (NHS) moiety. In some embodiments, the plurality of RCPs comprises modified nucleic acid residues. In some embodiments, amine-modified nucleic acid residues are incorporated during rolling circle amplification (RCA). In some embodiments, the solid support comprises an NHS ester reagent or a sulfo-NHS ester reagent that reacts with primary amines in the RCPs and/or primary amines in the particles, each reaction forming a stable conjugate comprising an amide bond. In some embodiments, the solid support is NHS ester- or sulfo-NHS ester-activated prior to contacting the RCPs and/or the particles.

In some embodiments, the plurality of RCPs comprises modified nucleic acid residues. In some embodiments, nucleic acid residues comprising tethering moieties are incorporated during rolling circle amplification (RCA). In some embodiments, the tethering moieties comprise functional groups (e.g., amines) or a binder of a binding pair, such as a biotin or a derivative or analog thereof, and the solid support comprises a binding partner with the binder (the binding partner and the binder forming the binding pair). In some embodiments, the binding partner comprises streptavidin, avidin, or a derivative or analog thereof. In some embodiments, amine-modified nucleic acid residues are incorporated during RCA. In some embodiments, amine-modified dNTP(s) is/are spiked in the dNTPs used for RCA.

In some embodiments, provided herein is a slide comprising a solid support which has rolling circle amplification products (RCPs) deposited thereon. In some embodiments, the kit comprises: rolling circle amplification products (RCPs) that are not in a cell or tissue; and a plurality of particles. In some aspects, provided herein is a slide comprising a solid support which has deposited thereon: rolling circle amplification products (RCPs), and a plurality of particles. In some embodiments, the RCPs and the particles are randomly deposited in one, two, three, four, or more discrete regions on the solid support.

In some embodiments, provided herein is a method for qualifying a system comprising an instrument, comprising: a) placing a solid support on the instrument, wherein the system comprises: the instrument, reagents comprising fluorescently labeled probes, a fluidics module, an optics module, and a system controller, and wherein the solid support comprises rolling circle amplification products (RCPs) deposited thereon, each RCP comprising multiple copies of an identifier sequence having an assigned signal code sequence from a codebook; b) using the fluidics module to deliver, in sequential cycles, the fluorescently labeled probes to the solid support; c) using the optics module to detect, in the sequential cycles, signals (or absence thereof) associated with the fluorescently labeled probes directly or indirectly bound to the identifier sequences in the RCPs, thereby generating signal code sequences for the RCPs (e.g., the signal code sequences can correspond to the identifier sequences (e.g., gene sequences or barcode sequences)); d) using the system controller to compare the generated signal code sequences to those from the codebook, thereby decoding the identifier sequences in the RCPs; and e) qualifying the system, wherein the system (e.g., operated in a workflow using the instrument and the reagents) is suitable for detecting analytes in a biological sample when the decoding in d) meets one or more pre-defined criteria.

In some embodiments, the identifier sequences in the RCPs comprise: sequences of a set of reference genes or complements thereof, and/or barcode sequences corresponding to a set of reference genes or complements thereof. In some embodiments, the identifier sequences in the RCPs comprise sequences of a set of reference genes or complements thereof. In some embodiments, the identifier sequences in the RCPs comprise barcode sequences corresponding to a set of reference genes or complements thereof. In some embodiments, the codebook comprises signal code sequences each corresponding to a reference gene of the set of reference genes. In some embodiments, the reagents comprise intermediate probes configured to hybridize to the identifier sequences in the RCPs, and the fluorescently labeled probes are configured to hybridize to the intermediate probes. In some embodiments, the number of different identifier sequences in the RCPs is at least 9, the number of fluorescently labeled probes of different sequences is 4, and the number of the sequential cycles is 4.

In some embodiments, the reagents for use on the instrument comprise intermediate probes configured to hybridize to the identifier sequences in the RCPs, and the fluorescently labeled probes are configured to hybridize to the intermediate probes. In some embodiments, the number of different identifier sequences in the RCPs is at least 9, the number of fluorescently labeled probes of different sequences is 4, and the number of the sequential cycles is 4. In some embodiments, the different identifier sequences are overlapping.

The solid support may but does not need to comprise a cell or tissue sample deposited thereon. In some embodiments, the solid support: (i) comprises a plurality of particles deposited thereon; and/or (ii) does not comprise a cell or tissue sample deposited thereon. In some embodiments, the solid support comprises a plurality of particles deposited thereon. In some embodiments, one or more of the plurality of particles are each coupled to one or more fluorescent moieties. In some embodiments, the fluorescent moiety has a maximum excitation wavelength of about 350 nm. In some embodiments, the fluorescent moiety comprises a blue fluorescent dye. In some embodiments, the particles are beads coupled to a blue fluorescent dye. In some embodiments, the RCPs and the plurality of particles are deposited in two, three, four, or more discrete regions on the solid support. In some embodiments, one or more of the discrete regions each comprises one or more fiducial markers.

In some embodiments, the sizes and/or shapes of the particles are comparable to cell nuclei in a cell or tissue sample. In some embodiments, the cell or tissue sample is analyzed using the qualified instrument or is a reference sample that is not analyzed using the qualified instrument. In some embodiments, the mean diameter of the particles is about 2 μm and the particles are round or oval. In some embodiments, the mean diameter of the particles is about 5 μm and the particles are round or oval. In some embodiments, any one or more of the particles is/are of a shape mimicking a cell, such as a round or oval shape. In some embodiments, the signal intensity of an individual particle (e.g., which functions as an artificial nucleus of a virtual cell for qualifying an instrument) on the solid support is comparable to the signal intensity of a nucleus detected in the biological sample (e.g., a cell or tissue sample). In some embodiments, the ratio between the number of the particles and the number of the RCPs is between about 1:5 and about 1:5,000. In some embodiments, the ratio between the number of the particles and the number of the RCPs is about 1:1,000.

In some embodiments, herein is provided a method for qualifying a system comprising an instrument, comprising: a) placing a solid support on the instrument, wherein the system comprises: the instrument, reagents comprising fluorescently labeled probes, a fluidics module, an optics module, and a system controller, and wherein the solid support comprises a plurality of particles deposited thereon, wherein the plurality of particles comprise rolling circle amplification products (RCPs) and each RCP comprises multiple copies of an identifier sequence having an assigned signal code sequence from a codebook; b) using the fluidics module to deliver, in sequential cycles, the fluorescently labeled probes to the solid support; c) using the optics module to detect, in sequential cycles, signals (or absence thereof) associated with the fluorescently labeled probes directly or indirectly bound to the identifier sequences in the RCPs, thereby generating signal code sequences for the RCPs; d) using the system controller to compare the generated signal code sequences to those from the codebook, thereby decoding the identifier sequences in the RCPs; and e) qualifying the system, wherein the system is suitable for detecting analytes in a biological sample when the decoding in d) meets one or more pre-defined criteria.

In some embodiments, the instrument comprises a sample module configured to receive and/or secure the solid support and/or the biological sample. In some embodiments, the instrument comprises an ancillary module configured to facilitate operation of the instrument. In some embodiments, the ancillary module comprises a cooling system and/or a motion calibration system. In some embodiments, the system controller controls operation of the fluidics module, the optics module, the sample module, and/or the ancillary module. In some embodiments, the system controller comprises a processor, a computer, and/or a computing platform. In some embodiments, the processor, the computer, and/or the computing platform are integrated. In some embodiments, the processor, the computer, and/or the computing platform comprises separate components configured to communicate with one another via a network. In some embodiments, the system controller comprises or is configured to communicate with a cloud computing platform. In some embodiments, the system controller is communicatively coupled with a data storage, an input device, a display system, or a combination thereof.

In some embodiments, the plurality of particles comprises a plurality of detectably labeled oligonucleotides. In some embodiments, the detectable labels of the detectably labeled oligonucleotides comprise a fluorescent dye. In some embodiments, the detectable labels comprise a blue fluorescent dye. In some embodiments, the detectably labeled oligonucleotide is hybridized to an anchor sequence of an anchor nucleotide conjugated to the particle. In some embodiments, the plurality of particles comprise DAPI beads encapsulated in a hydrogel. In some embodiments, the diameters of the plurality of particles are between about 1 μm and about 50 μm. In some embodiments, the mean diameter of the plurality of particles is about 10 μm. In some embodiments, the solid support comprises an additional plurality of particles deposited thereon. In some embodiments, the additional plurality of particles comprises beads directly or indirectly coupled to detectable labels. In some embodiments, the detectable labels comprise a fluorescent dye. In some embodiments, the detectable labels comprise a blue fluorescent dye. In some embodiments, the additional plurality of particles comprises a plurality of detectably labeled oligonucleotides. In some embodiments, the detectably labeled oligonucleotide comprises a fluorescent dye. In some embodiments, the detectable labels comprise a blue fluorescent dye. In some embodiments, the detectably labeled oligonucleotide is hybridized to an anchor sequence of an anchor oligonucleotide conjugated to the additional plurality of particles. In some embodiments, the detectably labeled oligonucleotides comprise a region of double stranded DNA dyed by DAPI. In some embodiments, the additional plurality of particles comprise DAPI beads encapsulated in a hydrogel. In some embodiments, the diameters of an additional plurality of particles are between about 1 μm and about 50 μm. In some embodiments, the mean diameter of the additional plurality of particles is about 10 μm. In some embodiments, the plurality of particles and/or the additional plurality of particles comprise hydrogel beads. In some embodiments, the RCPs are tethered to the plurality of particles via an anchor oligonucleotide comprising a primer sequence that has been used to generate the RCPs. In some embodiments, a portion of the anchor oligonucleotide is hybridized to a detectably labeled oligonucleotide. In some embodiments, the plurality of particles comprise functional groups. In some embodiments, the plurality of particles comprise functionalized gel beads comprise acrydite moieties. In some embodiments, a plurality of additional RCPs that are not detectable using detectably labeled probes is tethered to the plurality of particles.

In some embodiments, the RCPs are generated in solution outside a cell or tissue sample. In some embodiments, the identifier sequences in the RCPs comprise sequences of a set of reference genes or complements thereof, and/or barcode sequences corresponding to a set of reference genes or complements thereof. In some embodiments, the codebook comprises signal code sequences each corresponding to a reference gene of the set of reference genes.

In some embodiments, the reagents comprise intermediate probes configured to hybridize to the identifier sequences in the RCPs, and the fluorescently labeled probes are configured to hybridize to the intermediate probes. In some embodiments, the RCPs are hybridized to a detectably labeled oligonucleotide. In some embodiments, the detectably labeled oligonucleotides comprise a fluorescent dye. In some embodiments, the detectable labels comprise a blue fluorescent dye. In some embodiments, the detectably labeled oligonucleotides comprise a region of double stranded DNA dyed by DAPI.

In some embodiments, the instrument comprises a sample module configured to receive (and, optionally, secure) the solid support and/or the biological sample. In some embodiments, the instrument comprises an ancillary module configured to facilitate operation of the instrument. In some embodiments, the ancillary module comprises a cooling system and/or a motion calibration system. In some embodiments, the system controller controls operation of the fluidics module, the optics module, the sample module, and/or the ancillary module. In some embodiments, the system controller comprises a processor, a computer, and/or a computing platform. In some embodiments, the processor, the computer, and/or the computing platform are integrated, or are separate components configured to communicate with one another via a network. In some embodiments, the system controller comprises or is configured to communicate with a cloud computing platform. In some embodiments, the system controller is communicatively coupled with a data storage, an input device, a display system, or a combination thereof. In some embodiments, the biological sample is a cell or tissue sample.

In some embodiments, the biological sample is a cell or tissue sample. In some embodiments, the biological sample is a tissue section. In some embodiments, the biological sample is a matrix-embedded biological sample. In some embodiments, the biological sample is a cleared biological sample.

In some embodiments, the one or more pre-defined criteria comprise that at least or about 80%, at least or about 90%, or at least or about 95% of different identifier sequences in the RCPs are decoded using the instrument. In some embodiments, at least 90% of different identifier sequences in the RCPs are decoded using the instrument, and the instrument is used to detect analytes in the biological sample.

In some embodiments, the one or more pre-defined criteria comprise any one or more of: at least or about 35% of detected RCPs are decoded; the number of detected RCPs per μm² with a quality score of at least 20 is greater than or about 0.0001; the thickness of detected RCPs with a quality score of at least 20 is less than or about 1 μm; the maximum decoding false positive rate is less than or about 20%; the maximum decoding false negative rate is less than or about 80%; the minimal number of detected RCPs that are decoded with a quality score of at least 20 per field of view (FOV) is greater than 0; the quartile coefficient of dispersion of decoded different identifier sequences with a quality score of at least 20 per field of view (FOV) is less than or about 65%; and the number of particles detected is at least or about 400.

In some embodiments, the one or more pre-defined criteria comprises any one or more of: at least or about 40% of detected RCPs are decoded; the number of detected RCPs per μm² with a quality score of at least 20 is greater than or about 0.0003; the thickness of detected RCPs with a quality score of at least 20 is less than or about 0.8 μm; the maximum decoding false positive rate is less than or about 15%; the maximum decoding false negative rate is less than or about 90%; the minimal number of detected RCPs that are decoded with a quality score of at least 20 per field of view (FOV) is 1 or greater; the quartile coefficient of dispersion of decoded different identifier sequences with a quality score of at least 20 per field of view (FOV) is less than or about 65%; and the number of virtual cells detected based on detection of the particles is at least or about 500.

In some embodiments, the one or more pre-defined criteria comprises any one or more of: at least or about 29% of detected RCPs are decoded; the number of detected RCPs per μm² with a quality score of at least 20 is greater than or about 0.0003; the thickness of detected RCPs with a quality score of at least 20 is less than or about 0.8 μm; the maximum decoding false positive rate is less than or about 15%; the maximum decoding false negative rate is less than or about 90%; the minimal number of detected RCPs that are decoded with a quality score of at least 20 per field of view (FOV) is 1 or greater; the quartile coefficient of dispersion of decoded different identifier sequences with a quality score of at least 20 per field of view (FOV) is less than or about 60%; and the number of particles detected is at least or about 500.

In some aspects, provided herein is a method of qualifying a system comprising an instrument, comprising: a) placing a solid support on the instrument, wherein the system comprises: the instrument, reagents comprising intermediate probes and fluorescently labeled probes, a fluidics module, an optics module, and a system controller, and wherein the solid support comprises rolling circle amplification products (RCPs) immobilized thereon, each RCP comprising multiple copies of an identifier sequence having an assigned signal code sequence from a codebook; b) in a first cycle, using the fluidics module to deliver to the solid support a first plurality of intermediate probe/fluorescently labeled probe pairs, wherein the intermediate probe and the fluorescently labeled probe in each pair form a complex comprising the intermediate probe hybridized to an RCP of the plurality of RCPs and the fluorescently labeled probe hybridized to the intermediate probe, wherein the intermediate probe comprises (i) a recognition sequence complementary to the identifier sequence in the RCP and (ii) an overhang sequence, and wherein the fluorescently labeled probe comprises (i) a sequence complementary to the overhang sequence and (ii) a fluorescent label; c) using the optics module to detect first signals (or absence thereof) associated with the fluorescent labels of the first plurality of probe pairs at multiple locations on the solid support, wherein the first signal or absence thereof detected at a particular location corresponds to a first signal code in the signal code sequence assigned to the identifier sequence in the RCP at the particular location; d) in a second cycle, using the fluidics module to deliver to the solid support a second plurality of intermediate probe/fluorescently labeled probe pairs, wherein the intermediate probe and the fluorescently labeled probe in each pair form a complex comprising the intermediate probe hybridized to an RCP of the plurality of RCPs and the fluorescently labeled probe hybridized to the intermediate probe, wherein the intermediate probe comprises (i) a recognition sequence complementary to the identifier sequence in the RCP and (ii) an overhang sequence, and wherein the fluorescently labeled probe comprises (i) a sequence complementary to the overhang sequence and (ii) a fluorescent label; e) using the optics module to detect second signals (or absence thereof) associated with the fluorescent labels of the second plurality of probe pairs at multiple locations on the solid support, wherein the second signal or absence thereof detected at a particular location corresponds to a second signal code in the signal code sequence assigned to the identifier sequence in the RCP at the particular location, thereby generating a signal code sequence comprising at least the first signal code and the second signal code at each of the multiple locations; f) using the system controller to compare the generated signal code sequences for the RCPs at the multiple locations to those from the codebook, thereby decoding the identifier sequences in the RCPs; and g) qualifying the system for detecting analytes in a biological sample based on the decoding in f).

In some embodiments, in the first cycle, a first pool of intermediate probes and a universal pool of fluorescently labeled probes are delivered to the solid support, wherein each intermediate probe in the first pool of intermediate probes comprises (i) a recognition sequence complementary to one of the different identifier sequences in the RCPs, and (ii) a hybridization sequence complementary to a fluorescently labeled probe of the universal pool. In some embodiments, in the second cycle, a second pool of intermediate probes and the universal pool of fluorescently labeled probes are delivered to the solid support, wherein each intermediate probe in the second pool of intermediate probes comprises (i) a recognition sequence complementary to one of the different identifier sequences in the RCPs, and (ii) a hybridization sequence complementary to a fluorescently labeled probe of the universal pool. In some embodiments, the number of different identifier sequences in the RCPs is at least 9 and the number of fluorescently labeled probes of different sequences in the universal pool is 4.

In some embodiments, each fluorescently labeled probe of a different sequence in the universal pool is labeled with a fluorophore of a different color. In some embodiments, the signal code sequence comprises the first signal code, the second signal code, a third signal code corresponding to a third cycle, and a fourth signal code corresponding to a fourth cycle. In some embodiments, the signal code sequence comprises a dark signal code corresponding to the absence of signal in the corresponding cycle.

In some embodiments, when at least or about 90% of the different identifier sequences in the RCPs are decoded using the instrument, the system is determined to pass the qualification test as suitable for decoding analytes in situ in the biological sample. In some embodiments, the method comprises using the qualified system (e.g., instrument and reagents provided thereon) to decode analytes in situ in the biological sample.

In some aspects, the RCPs are attached to a plurality of gel beads and the gel beads are immobilized on the solid support. In some embodiments, the plurality of gel beads comprise a primer oligonucleotide for generating the RCPs.

In some embodiments, provided herein is a method for producing a slide, comprising: a) separately generating rolling circle amplification products (RCPs) of each circular template of a plurality of different circular templates, wherein the RCPs of each circular template comprise multiple copies of a different identifier sequence which is assigned a different signal code sequence from a codebook; b) pooling RCPs of the plurality of different circular templates; c) disposing the pooled RCPs on a solid support, thereby producing the slide comprising the solid support and the pooled RCPs thereon. In some embodiments, the method comprises disposing a plurality of particles on the solid support prior to, concurrently with, or after disposing the pooled RCPs on the solid support. In some embodiments, the particles comprise latex beads or hydrogel beads coupled to a blue fluorescent dye.

In some embodiments, the RCPs of each circular template are generated outside a cell or tissue sample, generated in solution, and/or generated in vitro. In some embodiments, the RCPs are generated on a plurality of gel beads and the step of disposing the pooled RCPs on a solid support, thereby producing the slide comprising the solid support and the pooled RCPs thereon, comprises disposing the plurality of gel beads on the solid support. In some embodiments, two or more different circular templates are amplified in the same solution using rolling circle amplification. In some embodiments, the plurality of gel beads are disposed on the solid support in a buffer comprising a surfactant. In some embodiments, each one of a plurality of different circular templates are amplified in a separate solution, and the separate solutions can be pooled. In some embodiments, the surfactant is an anionic surfactant. In some embodiments, the buffer comprises sodium lauroyl sarcosinate.

In some embodiments, the method comprises providing or generating each circular template separately in a solution. In some embodiments, the circular template is generated by hybridizing a circularizable probe or probe set to a splint oligonucleotide, and circularizing the circularizable probe or probe set using the splint oligonucleotide as a template. In some embodiments, the circularizing comprises ligation templated on the splint oligonucleotide, with or without gap filling prior to the ligation. In some embodiments, the solution comprising the circular template is diluted prior to rolling circle amplification of the circular template. In some embodiments, the RCPs of each circular template comprise modified nucleic acid residues. In some embodiments, nucleic acid residues comprising tethering moieties are incorporated during rolling circle amplification. In some embodiments, the nucleic acid residues are amine-modified or comprise a biotin or a derivative or analog thereof.

In some embodiments, any one of more of the circular templates are amplified using rolling circle amplification in a solution and outside a cell or tissue sample. In some embodiments, two or more different circular templates are amplified in the same solution using rolling circle amplification. In some embodiments, each one of a plurality of different circular templates are amplified in a separate solution, and the separate solutions are pooled. In some embodiments, the method comprises providing or generating each circular template separately in a solution. In some embodiments, the circular template is generated by hybridizing a circularizable probe or probe set to a splint oligonucleotide, and circularizing the circularizable probe or probe set using the splint oligonucleotide as a template. In some embodiments, the circularizing comprises ligation templated on the splint oligonucleotide, with or without gap filling prior to the ligation. In some embodiments, the solution comprising the circular template is diluted prior to rolling circle amplification of the circular template.

In some embodiments, the RCPs of each circular template comprise modified nucleic acid residues. In some embodiments, one or more nucleic acid residues comprising tethering moieties are incorporated during rolling circle amplification. In some embodiments, one or more amine-modified nucleic acid residues are incorporated during rolling circle amplification.

In some embodiments, the solid support comprises functional groups configured to bind to or react with the tethering moieties, thereby immobilizing the RCPs on the solid support. In some embodiments, the solid support comprises functional groups configured to bind to or react with one or more modified nucleic acid residues, thereby immobilizing RCPs comprising modified nucleic acid residues on the solid support. In some embodiments, the functional groups comprise an N-hydroxysuccinimide (NHS) moiety configured to react with amines in the RCPs and/or in the particles.

In some embodiments, the ratio between the number of the particles and the number of the RCPs is between about 1:5 and about 1:5,000. In some embodiments, the ratio between the number of the particles and the number of the RCPs is about 1:1,000.

In some embodiments, the RCPs are pooled outside a cell or tissue. In some embodiments, the pooled RCPs are not in a cell or tissue. In some embodiments, any two or more of the RCPs are pooled in solution, dried (e.g., freeze-dried), or in lyophilized form. In some embodiments, the pooled RCPs are deposited in two, three, four, or more discrete regions on the solid support. In some embodiments, one or more of the discrete regions each comprises one or more fiducial markers. In some embodiments, the solid support comprises one or more fiducial markers. In some embodiments, any two or more of the RCPs are pooled in solution. In some embodiments, any two or more of the RCPs are pooled as dried (e.g., freeze-dried) RCPs. In some embodiments, any two or more of the RCPs are pooled in lyophilized form. In some embodiments, any two or more of the RCPs are pooled in dried form. In some embodiments, the pooled RCPs are not in a cell or tissue. In some embodiments, the RCPs are pooled and deposited on a solid support that is free of cells. In some embodiments, the pooled and deposited RCPs are not in a cell or tissue.

In some embodiments, the pooled RCPs are deposited in any one or more discrete regions at any location on the solid support, for instance, at or near a corner or at or near the center of the solid support. In some embodiments, the pooled RCPs are deposited in two, three, four, or more discrete regions on the solid support, for instance, at the four corners of a surface area on the solid support. In some embodiments, the solid support comprise one or more fiducial markers.

In some embodiments, provided herein is a kit, comprising: a particle comprising: i) a rolling circle amplification product (RCP) and ii) a fluorescent dye; and a solid support comprising a functional group for immobilizing the particle on the solid support.

In some embodiments, provided herein is a kit, comprising: a plurality of particles, wherein a particle of the plurality of particles comprises: i) a rolling circle amplification product (RCP) and ii) a fluorescent dye; and a solid support comprising functional groups for immobilizing the plurality of particles on the solid support. In some embodiments, the fluorescent dye is a blue fluorescent dye. In some embodiments, the plurality of particles comprises gel beads. In some embodiments, the plurality of particles comprises beads coupled to a plurality of detectably labeled oligonucleotides comprising the blue fluorescent dye. In some embodiments, the blue fluorescent dye has an excitation wavelength between about 300 nm and about 400 nm. In some embodiments, a detectably labeled oligonucleotide of the plurality of detectably labeled oligonucleotides is hybridized to an anchor sequence of an anchor oligonucleotide conjugated to a particle of the plurality of particles. In some embodiments, a detectably labeled oligonucleotide of the detectably labeled oligonucleotides comprises a region of double stranded DNA dyed by DAPI. In some embodiments, the RCP is hybridized to a detectably labeled oligonucleotide comprising the blue fluorescent dye. In some embodiments, the RCP hybridized to the detectably labeled oligonucleotide is dyed with DAPI. In some embodiments, the anchor oligonucleotide comprises the primer sequence used for generating the RCP. In some embodiments, the diameters of the plurality of particles are between about 1 μm and about 50 μm. In some embodiments, the mean diameter of the plurality of particles is about 10 μm. In some embodiments, the plurality of particles comprise DAPI beads encapsulated in a hydrogel.

In some embodiments, provided herein is a kit, comprising: a plurality of particles, wherein a particle of the plurality of particles comprises a rolling circle amplification product (RCP); an additional plurality of particles comprising a fluorescent dye, and a solid support comprising functional groups for immobilizing the plurality of particles and the additional plurality of particles on the solid support. In some embodiments, the fluorescent dye is a blue fluorescent dye. In some embodiments, the additional plurality of particles comprises gel beads. In some embodiments, the blue fluorescent dye has an excitation wavelength between about 300 nm and about 400 nm. In some embodiments, a particle of the additional plurality of particles comprises a plurality of oligonucleotides conjugated to the blue fluorescent dye. In some embodiments, a particle of the additional plurality of particles comprises beads coupled to a plurality of detectably labeled oligonucleotides comprising the blue fluorescent dye. In some embodiments, the detectably labeled oligonucleotides are hybridized to an anchor sequence of an anchor oligonucleotide conjugated to the additional plurality of particles. In some embodiments, the additional plurality of particles comprise DAPI beads encapsulated in a hydrogel. In some embodiments, the diameters of the additional plurality of particles are between about 1 μm and about 50 μm. In some embodiments, the mean diameter of the additional plurality of particles is about 10 μm. In some embodiments, the detectably labeled oligonucleotides comprise a region of double stranded DNA dyed by DAPI. In some embodiments, the plurality of particles and/or the additional plurality of particles comprise hydrogel beads. In some embodiments, the RP is generated in solution outside a cell or tissue sample. In some embodiments, the functional group is attached to a primer oligonucleotide for generating the RCP. In some embodiments, the plurality of particles comprise functionalized gel beads comprising acrydite moieties.

In some embodiments, the RCP comprises multiple copies of an identifier sequence and the identifier sequence in the RCPs corresponds to a reference gene or complements thereof. In some embodiments, the kit further comprises a plurality of intermediate probes configured to hybridize to the identifier sequences in the RCPs. In some embodiments, the kit further comprises a plurality of fluorescently labeled probes configured to hybridize to the intermediate probes. In some embodiments, the functional groups bind to or react with tethering moieties in the plurality of particles and/or the additional plurality of particles, thereby immobilizing the RCPs and/or the fluorescent dyes on the solid support. In some embodiments, a plurality of additional RCPs that are not detectable using detectably labeled probes is tethered to the plurality of particles.

### BRIEF DESCRIPTION OF THE DRAWINGS

The drawings illustrate certain features and advantages of this disclosure. These embodiments are not intended to limit the scope of the appended claims in any manner.

FIG. 1 is an example of a workflow of using an opto-fluidic instrument, according to various embodiments. The workflow can be used for in situ analysis and/or an instrument performance test disclosed herein. The sample can be a QC slide disclosed herein for assessing instrument performance or a biological sample (e.g., a cell or tissue sample for in situ analyte detection).

FIG. 2 is an example of a workflow of an instrument performance test. Synthetic constructs such as rolling circle amplification (RCA) products (RCPs) comprising barcode sequences can be generated in vitro and deposited on a slide, together with particles (e.g., beads conjugated to a blue fluorescent dye) that mimic nuclei stained with DAPI, in order to generate a QC slide. The RCPs can be generated using pairs of circularizable probe (e.g., padlock (“PD”) probe shown in figure) and splint oligonucleotide (e.g., “DNA oligo” shown in figure). An instrument can be used to cycle detectably labeled oligonucleotides for detecting the synthetic constructs on the QC slide, and the barcode sequences in the RCPs can be decoded based on the signals detected in sequential probe hybridization cycles. Metrics associated with decoding the QC slide can be used to evaluate performance of the instrument and the assay.

FIG. 3A shows an example QC slide with RCPs deposited in regions of a solid support and a codebook for decoding Genes 1-9 through sequence probe hybridization Cycles 1-4. FIG. 3B shows a representative fluorescent microscopy image of the QC slide, showing RCPs were decoded as genes (e.g., transcripts) and fluorescently labeled particles (e.g., beads conjugated with a blue fluorescent dye) were segmented as cells. Based on the detection of signals associated with the RCPs and signals associated with nuclei-mimicking nuclei, the RCPs on the QC slide were decoded. FIG. 3C shows gene counts of Genes 1-9 detected on the QC slide, as compared to a negative control (“dummy”).

FIG. 4 is an example of a workflow for generating synthetic constructs such as rolling circle amplification (RCA) products (RCPs) in vitro on gel beads and deposited on a slide, in order to generate a QC slide. A bead can be functionalized with an oligonucleotide (e.g., TTTTTTTTTTGGCTCCACTAAATAGACGCA, SEQ ID No: 1) comprising an acrydite moiety.

FIG. 5 shows signals detected from RCPs generated on gel beads deposited on a slide compared to signals detected from RCPs generated in cell pellet sample comprising cells from two different cell lines.

FIG. 6 shows a plot of RCP count (“transcript count”) detected at various concentrations of circularized probes associated with a panel of reference genes.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

Profiling analytes in biological samples in situ (e.g., transcriptomic profiling) using microscopic imaging provides valuable information regarding analyte abundance and localization in situ. There is a need for methods to assess performance of in situ analysis platforms (e.g., methods, assays, workflows, and/or instruments for in situ analyte detection) in various scenarios. For example, the performance of an instrument (e.g., one that comprises a microscope) might be assessed as a quality control measure to ensure that the reagents, fluidics, optics, processors, and/or other modules, as well as the instrument as a whole, are working as expected and producing reliable data. In another example, performance of multiple instrument configurations is compared. For instance, instruments comprising different optics modules and/or fluidics modules may perform differently, and a particular configuration of fluidics may work better in conjunction with a particular configuration of optics than with other optics module configurations. However, assessing and/or comparing the performance of instrument configurations can be challenging, in part because the results are dependent on the sample used for the assessment, and biological samples such as cells and tissue sections can have high sample-to-sample variance.

For instance, an in situ workflow may comprise many steps upstream of steps performed on an instrument using components of the instrument. These upstream steps can include sample collection, shipping, storage, sectioning, fixing (e.g., for fresh frozen samples), deparaffinization (e.g., for FFPE samples), permeabilization, crosslinking/de-crosslinking, probe hybridization, washing (e.g., stringency washes), probe ligation, primer hybridization, amplification (e.g., RCA), and/or binding of detection agents (e.g., staining and/or antibody binding). Tissue samples can present particular challenges when used for quality control (QC) of instrument performance, since the QC tissue samples are typically subjected to various upstream steps. Tissue intrinsic variations (e.g., due to different tissue types or cellular heterogeneity even in the same block of tissue), as well as variations introduced by any one or more of the upstream steps before the QC tissues are imaged and decoded, may make it difficult to interpret QC results and provide reliable assessment of instrument performance.

In some aspects, provided herein are in vitro methods, compositions, kits, and systems for QC of an instrument prior to analyzing cells or tissues using the instrument. In some embodiments, provided herein is a test slide (e.g., a decoding QC slide) having in vitro synthesized nucleic acid molecules (e.g., RCPs) that mimic products of library preparation performed in situ in a cell or tissue sample. As such, the test slide is independent of library preparation in biological samples and can be cell-free and tissue-free. For instance, the test nucleic acid molecules (e.g., RCPs) can be synthesized in solution using circular or circularizable probes and artificial target nucleic acids of the probes, and immobilized on a slide to generate the test slide. In some embodiments, the test nucleic acid molecules (e.g., RCPs) are synthesized on a functionalized particle (e.g., a gel bead) using circular or circularized probes and deposited on a slide to generate the test slide. Using the test slide can decouple instrument performance from upstream steps (e.g., probe hybridization, ligation, and/or RCA) which may be necessary if cell or tissue samples are used as QC test samples, and from biologically variable sample types, providing a short, simplified and reliable workflow for assessing instrument performance.

The test slides (e.g., decoding QC slides) can be produced in large numbers and stored for long periods of time compared to QC slides having cell or tissue samples. In some aspects, the test slides are used to produce reproducible assay results. In some embodiments, synthetic constructs described herein provided on a slide for detection are generated in vitro and then attached to a slide, reducing preparation time compared to RCPs generated on the slide. In some embodiments, synthetic constructs described herein require reduced preparation and/or resources compared to RCPs generated in a cell or tissue sample. In some embodiments, synthetic constructs that are or act as RCPs are provided on a slide for detection and decoding on an instrument using the standard reagents and workflow, and if it passes instrument QC, the instrument is used for in situ analyte detection in a cell or tissue sample. In some embodiments, if the instrument fails QC, one or more configurations of the instrument, various modules of the instrument, reagents provided in/on the instrument, and/or methods of operating the instrument are adjusted, and the adjusted instrument is assessed for one or more times until it passes QC. This way, the QC tests can be performed efficiently and reliably.

### II. Quality Control (Qc) Slides, Kits, and Manufacturing Methods

In some aspects, provided herein are quality control slides comprising a plurality of nucleic acid molecules such as RCPs that function as standardized test molecules to be detected and decode using an instrument; a plurality of particles (e.g., fluorescently labeled beads) that function as artificial nuclei; and a solid support comprising functional groups for immobilizing the plurality of nucleic acid molecules and/or the plurality of particles on the solid support. In some aspects, provided herein are quality control slides comprising a plurality of nucleic acid molecules such as RCPs that function as standardized test molecules suitable for training slides for performing an in situ assay as described in Section V.

In some aspects, provided herein are compositions and kits comprising any one of the quality control slides disclosed herein. In some embodiments, a kit disclosed herein comprises instructions for performing any one of the methods disclosed herein for analyzing performance of an instrument for in situ analyte detection.

In some embodiments, the kit comprises reagents and/or consumables required for performing one or more steps of the provided methods. In some aspects, the kit comprises any one of the quality control slides disclosed herein. In some embodiments, the kit comprises reagents, such as enzymes and buffers for ligation and/or amplification, such as ligases and/or polymerases. In some aspects, the kit can also comprise any one of the reagents described herein, e.g., wash buffer and ligation buffer. In some embodiments, the kit comprises reagents for detection, such as detectably labeled probes and intermediate probes disclosed herein. In some embodiments, the kit comprises other components, for example nucleic acid primers, enzymes and reagents, buffers, nucleotides, and reagents for additional assays. In some embodiments, the kit consists of a quality control slide disclosed herein and packaging and optionally instruments for using the quality control slide, and other components such as probes, enzymes, and buffers are not provided with the kit. In some aspects, various components (e.g., reagents, enzymes and buffers for ligation and/or amplification, probes for detection) are provided separately from the kit and the quality control slide(s) therein. The various components of the kit may be present in separate containers or certain compatible components may be pre-combined into a single container. In some embodiments, the kits further contain instructions for using the components of the kit to practice the provided methods.

### A. QC Slides and Kits

In some aspects, provided herein is a kit comprising: a plurality of synthetic constructs such as rolling circle amplification products (RCPs); a plurality of particles; and a solid support comprising functional groups for immobilizing the plurality of synthetic constructs and/or the plurality of particles on the solid support. In some embodiments, the plurality of synthetic constructs and/or the plurality of particles are immobilized on the solid support. In some embodiments, instead of providing the plurality of synthetic constructs as components of the kit, the kit comprises reagents for generating the synthetic constructs such as RCPs. Likewise, in some embodiments, instead of providing the plurality of particles as components of the kit, the kit comprises reagents for generating the particles such as fluorescently labeled beads. In some embodiments, the kit comprises instructions for depositing (e.g., immobilizing) the synthetic constructs and/or particles on the solid support.

The synthetic constructs that are or act as RCPs can be coupled to the solid support (e.g., a QC slide) using a variety of attachment methods using chemical and/or enzymatic interactions/reactions. The RCPs can be synthesized, for example, using circularizable probes (e.g., padlock probes) or engineered plasmids that do not require templates. Synthetic constructs having around the same size as an RCP and contains binding sites for intermediate probes (e.g., L-shaped probes) that correspond to a gene in a codebook can be used. In some embodiments, the RCPs are embedded within a hydrogel on the QC slide. In some cases, the synthetic constructs that are or act as RCPs are generated on and coupled to a bead (e.g., a gel bead), and beads with the synthetic constructs generated thereon are deposited on a surface of a substrate, such as a slide.

In some embodiments, a kit comprises a plurality of particles, wherein a particle of the plurality of particles comprises a rolling circle amplification product (RCP); an additional plurality of particles comprising a fluorescent dye, and a solid support comprising functional groups for immobilizing the plurality of particles and the additional plurality of particles on the solid support. In some embodiments, the additional plurality of particles comprises a fluorescent dye blue fluorescent dye. In some embodiments, a kit comprises a plurality of particles, wherein a particle of the plurality of particles comprises a rolling circle amplification product (RCP); an additional plurality of particles comprising a blue fluorescent dye, and a solid support comprising functional groups for immobilizing the plurality of particles and the additional plurality of particles on the solid support. In some embodiments, the kit comprises a plurality of particles each comprising an RCP and an additional plurality of particles each comprising a blue fluorescent dye, and the plurality of particles and the additional plurality of particles are different particles. In some embodiments, the additional plurality of particles are dyed beads.

### (i) Synthetic Constructs

In some embodiments, any one or more of the synthetic constructs (e.g., RCPs) are generated outside a cell or tissue sample. In some embodiments, any one or more of the synthetic constructs are generated in solution. In some embodiments, any one or more of the synthetic constructs are generated in vitro. In some embodiments, the synthetic constructs are not in a cell or tissue. In some embodiments, any one or more of the synthetic constructs are in solution. In some embodiments, any one or more of the synthetic constructs are lyophilized. In some embodiments, any one or more of the synthetic constructs are dried. In some embodiments, the kit comprises one or more vials, each vial comprising synthetic constructs in solution or lyophilized synthetic constructs. In some embodiments, the synthetic constructs in different vials are pooled. For instance, contents of a vial containing synthetic constructs in solution can be combined with another vial containing synthetic constructs in solution. In other instances, contents of a vial containing lyophilized synthetic constructs are combined with another vial containing lyophilized synthetic constructs. Synthetic constructs in solution can also be pooled with lyophilized or dried synthetic constructs.

In some embodiments, each synthetic construct of the plurality of synthetic constructs comprises multiple copies of an identifier sequence. In some embodiments, each synthetic construct (as well as the identifier sequence therein) is associated with an assigned signal code sequence from a codebook. In some embodiments, each different identifier sequence is assigned a different signal code sequence from a codebook. In some embodiments, each different identifier sequence and the synthetic construct comprising it are uniquely identified by a signal code sequence from a codebook. In some embodiments, the identifier sequence is associated with a gene or a product of the gene, and the identifier sequence comprises or is a barcode sequence that corresponds to the gene or product thereof.

In some embodiments, the synthetic construct comprises multiple copies of the identifier sequence. In some embodiments, the synthetic construct is a concatemeric synthetic construct with multiple copies of the identifier sequence. In some embodiments, the synthetic construct comprises between about 5 and about 50, between about 10 and about 100, between about 100 and about 1,000, between about 1,000 and about 5,000, between about 5,000 and about 10,000, or more than 10,000 copies of the identifier sequence. In some embodiments, the synthetic construct comprises between about 1,000 and about 5,000, between about 5,000 and about 10,000, or more than 10,000 copies of the identifier sequence.

In some embodiments, the synthetic construct is in the form of a nanoball. In some embodiments, the nanoball has a diameter of between about 0.1 μm and about 3 μm, e.g., between about 0.1 μm and about 0.5 μm (e.g., between about 0.2 μm and about 0.3 μm, or between about 0.3 μm and about 0.4 μm), between about 0.5 μm and about 1 μm, between about 0.8 μm and about 1.3 μm, or between about 1 μm and about 1.5 μm. In some embodiments, the nanoball has a diameter of between about 0.5 μm and about 1 μm, between about 0.8 μm and about 1.3 μm, or between about 1 μm and about 1.5 μm.

In some embodiments, the synthetic construct is between about 1 and about 15 kilobases, between about 15 and about 25 kilobases, between about 25 and about 35 kilobases, between about 35 and about 45 kilobases, between about 45 and about 55 kilobases, between about 55 and about 65 kilobases, between about 65 and about 75 kilobases, or more than 75 kilobases in length, e.g., between about 45 and about 70 kilobases. In some embodiments, the synthetic construct is between about 25 and about 35 kilobases, between about 35 and about 45 kilobases, between about 45 and about 55 kilobases, between about 55 and about 65 kilobases, between about 65 and about 75 kilobases, or more than 75 kilobases in length, e.g., between about 45 and about 70 kilobases.

In some embodiments, the synthetic construct is an amplification product. In some embodiments, the synthetic construct comprises an RCP, e.g., an RCP generated according to any one of the described methods, for instance an RCP of a circular or circularized probe. In some embodiments, the identifier sequence is a barcode sequence or complement thereof of the RCP.

In some embodiments, the RCA for generating the RCP of the synthetic construct is performed for about 10 minutes, about 15 minutes, about 20 minutes, about 30 minutes, about 45 minutes, about one hour, about two hours, about three hours, or longer.

In some aspects, one or more of the identifier sequences comprises one or more barcode(s), e.g., at least two, three, four, five, six, seven, eight, nine, ten, or more barcodes. Barcodes can be used to spatially resolve molecular components found in biological samples, for example, within a cell or a tissue sample, as well as synthetic constructs comprising barcodes, for example, RCPs provided on a QC slide disclosed herein. In some aspects, a barcode comprises about 4, 5, 6, 7, 8, 9, 10, 11, 12, 13, 14, 15, 16, 17, 18, 19, 20, 21, 22, 23, 24, 25, 26, 27, 28, 29, 30, or more than 30 nucleotides.

In some embodiments, a barcode comprises two or more sub-barcodes that together function as a single barcode. For example, a polynucleotide barcode can include two or more polynucleotide sequences (e.g., sub-barcodes) that are separated by one or more non-barcode sequences. In some embodiments, the one or more barcode(s) can also provide a platform for targeting functionalities, such as oligonucleotides, oligonucleotide-antibody conjugates, oligonucleotide-streptavidin conjugates, modified oligonucleotides, affinity purification, detectable moieties, enzymes, enzymes for detection assays or other functionalities, and/or for detection and identification of the polynucleotide.

In some embodiments, barcodes or complements thereof (e.g., barcode sequences or complements thereof comprised by the RCPs herein) are detected by sequential hybridization and detection with a plurality of probes (e.g., detectably labeled probes, or intermediate probes and detectably labeled probes targeting the intermediate probes) described herein. In some embodiments, barcode sequences are detected for identification of other molecules including nucleic acid molecules (DNA or RNA) longer than the barcode sequences themselves, as opposed to direct (e.g., base-by-base) sequencing of the longer nucleic acid molecules. In some embodiments, a N-mer barcode sequence comprises 4N complexity given a sequencing read of N bases, and a much shorter sequencing read may be required for molecular identification compared to non-barcode sequencing methods such as direct sequencing. For example, 1024 molecular species may be identified using a 5-nucleotide barcode sequence (45=1024), whereas 8 nucleotide barcodes can be used to identify up to 65,536 molecular species, a number greater than the total number of distinct genes in the human genome. In some embodiments, the barcode sequences contained in the probes or products thereof (e.g., RCPs) are detected, rather than endogenous sequences, which can be an efficient read-out in terms of information per cycle of sequencing. Because the barcode sequences are pre-determined, they can also be designed to feature error detection and correction mechanisms, see, e.g., U.S. Pat. Pub. 20190055594 and 20210164039, which are hereby incorporated by reference in their entirety.

In some embodiments, a synthetic construct (e.g., RCP) disclosed herein comprises 1, 2, 3, 4, 5, 6, 7, 8, 9, 10, 11, 12, 13, 14, 15 or more, 20 or more, 32 or more, 40 or more, or 50 or more different barcode sequences. The barcode sequences may be positioned anywhere within the synthetic construct. If more than one barcode sequences are present, the barcode sequences may be positioned next to each other, and/or interspersed with other sequences. In some embodiments, two or more of the barcode sequences may also at least partially overlap. In some embodiments, two or more of the barcode sequences in the same synthetic construct do not overlap. In some embodiments, all of the barcode sequences in the same synthetic construct are separated from one another by at least a phosphodiester bond (e.g., they may be immediately adjacent to each other but do not overlap), such as 1, 2, 3, 4, 5, 6, 7, 8, 9, 10 or more nucleotides apart.

The barcode sequences, if present, may be of any length. If more than one barcode sequence is used, the barcode sequences may independently have the same or different lengths, such as at least 5, at least 10, at least 15, at least 20, at least 25, at least 30, at least 35, at least 40, at least 50 nucleotides in length. In some embodiments, the barcode sequence is no more than 120, no more than 112, no more than 104, no more than 96, no more than 88, no more than 80, no more than 72, no more than 64, no more than 56, no more than 48, no more than 40, no more than 32, no more than 24, no more than 16, or no more than 8 nucleotides in length. Combinations of any one of these are also possible, e.g., the barcode sequence may be between 5 and 10 nucleotides, between 8 and 15 nucleotides, etc.

The barcode sequence may be arbitrary or random. In certain cases, the barcode sequences are chosen so as to reduce or minimize homology with other components on the QC slide, e.g., such that the barcode sequences do not themselves bind to or hybridize with other nucleic acids suspected of being on a QC slide. In some embodiments, between a particular barcode sequence and another sequence (e.g., other barcode sequences in different RCPs on a QC slide), the homology is less than 10%, less than 8%, less than 7%, less than 6%, less than 5%, less than 4%, less than 3%, less than 2%, or less than 1%. In some embodiments, the homology is less than 20, 19, 18, 17, 16, 15, 14, 13, 12, 11, 10, 9, 8, 7, 6, 5, 4, 3, or 2 bases, and in some embodiments, the bases are consecutive bases.

In some embodiments, the number of distinct barcode sequences in a population of synthetic constructs is less than the number of distinct targets (e.g., different genes encoded by different signal code sequences in a codebook), and yet the distinct targets may still be uniquely decoded and identified from one another, e.g., by encoding each barcode sequence with a different combination of intermediate probes and detectably labeled probes targeting the intermediate probes.

The synthetic constructs can be deposited in any region on the solid support. In some embodiments, the synthetic constructs are randomly deposited in one, two, three, four, or more regions on the solid support.

In some embodiments, the plurality of synthetic constructs (e.g., RCPs) comprise modified nucleic acid residues. In some embodiments, amine-modified nucleic acid residues are incorporated during rolling circle amplification (RCA). For example, amine-modified dNTP(s) can be spiked in the dNTPs used for RCA.

### (ii) Particles

In some embodiments, the plurality of particles comprises beads coupled to detectable labels. In some embodiments, the plurality of particles comprise beads coupled to oligonucleotides for generating synthetic constructs that are or act as RCPs as described in Section II.B. In some embodiments, the generated synthetic constructs (e.g., RCPs) are coupled to the bead. Each bead can be coupled to one or more molecules of the same detectable label or different detectable labels. In some embodiments, each particle is independently coupled to 2, 3, 4, 5, 6, 7, 8, 9, 10, or more molecules of the same fluorophore or different fluorophores.

In some embodiments, the size and/or shape of an individual particle is comparable to that of a nucleus in a cell or tissue sample. In some embodiments, the diameters of the particles is between about 0.5 μm and about 20 μm. In some embodiments, the diameter(s) of any one or more of the particles is/are about 0.5 μm, about 0.6 μm, about 0.7 μm, about 0.8 μm, about 0.9 μm, about 1.0 μm, about 1.1 μm, about 1.2 μm, about 1.3 μm, about 1.4 μm, about 1.5 μm, about 1.6 μm, about 1.7 μm, about 1.8 μm, about 1.9 μm, about 2.0 μm, about 2.1 μm, about 2.2 μm, about 2.3 μm, about 2.4 μm, about 2.5 μm, about 2.6 μm, about 2.7 μm, about 2.8 μm, about 2.9 μm, or about 3.0 μm. In some embodiments, at least 50%, at least 60%, at least 70%, at least 80%, at least 90%, at least 99%, or all of the particles have diameters between about 1.5 μm and about 2.5 μm. In some embodiments, the mean diameter of the particles is about 1.8 μm, about 2 μm, or about 2.2 μm.

In some embodiments, the size and/or shape of an individual particle is comparable to that of a cell. In some embodiments, the diameters of the particles are between about 1 μm and about 20 μm, between about 1 μm and about 50 μm, between about 1 μm and about 100 μm, between about 5 μm and about 50 μm, or between about 5 μm and about 20 μm. In some embodiments, the diameters of the particles are between about 2 μm and about 20 μm. In some embodiments, the diameter(s) of any one or more of the particles is about 5 μm, about 6 μm, about 7 μm, about 8 μm, about 9 μm, or about 10 μm. In some embodiments, at least 50%, at least 60%, at least 70%, at least 80%, at least 90%, at least 99%, or all of the particles have diameters between about 1 μm and about 50 μm. In some embodiments, at least 50%, at least 60%, at least 70%, at least 80%, at least 90%, at least 99%, or all of the particles have diameters between about 5 μm and about 15 μm. In some embodiments, the mean diameter of the particles is about 8 μm. In some embodiments, the mean diameter of the particles is about 10 μm. In some embodiments, the mean diameter of the particles is about 12 μm. In some embodiments, the diameter(s) of any one or more of the particles is about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, or about 20 μm. In some embodiments, at least 50%, at least 60%, at least 70%, at least 80%, at least 90%, at least 99%, or all of the particles have diameters between about 5 μm and about 15 μm. In some embodiments, the mean diameter of the particles is about 8 μm, about 10 μm, or about 12 μm.

In some embodiments, a particle of the plurality of particles can have a size and/or shape mimicking that of a cell, e.g., a cell in a tissue section of analysis using a microscope. In some embodiments, the plurality of particles comprise round beads and/or oval beads, e.g., beads coupled to fluorescent moieties such as blue fluorescent dyes. In some embodiments, the signal intensity of detectable labels on an individual bead is comparable to the signal intensity of a nucleus detected in a cell or tissue sample. In some embodiments, the size, shape, and/or signal intensity of an individual particle is such that the particle is segmented using microscopy as an individual nucleus of a virtual cell. In some embodiments, a cell segmentation algorithm is applied on the detected particles. In some cases, the cell segmentation comprises performing nuclear expansion based on the detected blue fluorescent dye associated with beads. For example, boundaries are expanded from each particle (e.g., beads coupled to blue fluorescent dye) until expanded boundaries of neighboring particle touch (e.g., to mimic cell boundaries). In some aspects, any suitable cell segmentation algorithm such as cellpose (cellpose.org) or omnipose (Culter et al., Nature Methods volume 19, pages 1438-1448 (2022) incorporated herein by reference) can be used.

In some embodiments, the detectable labels in the particles can have an excitation wavelength between about 300 nm and about 800 nm. In some embodiments, the detectable labels in the particles can have an excitation wavelength between about 300 nm and about 400 nm. In some embodiments, the detectable labels in the particles can have an excitation wavelength of about 310 nm, about 320 nm, about 330 nm, about 340 nm, about 350 nm, about 360 nm, about 370 nm, about 380 nm, or about 390 nm. In some embodiments, the detectable labels have a maximum excitation wavelength of about 360 nm. In some embodiments, the detectable labels in the particles are non-autofluorescent. In some embodiments, the detectable labels in the particles are substantially nonfluorescent under excitation wavelengths greater than about 420 nm. In some embodiments, the detectable labels in the particles are substantially nonfluorescent under excitation wavelengths between about 450 nm and about 750 nm. In some embodiments, the detectable labels in the particles are substantially nonfluorescent under excitation wavelengths of about 490 nm, about 530 nm, about 590 nm, and/or about 650 nm.

In some embodiments, the detectable labels are labels that fluoresce in a detectable wavelength in the UV-far red spectrum. A blue fluorophore can be used and in some embodiments is selected for convenience.

In some embodiments, the detectable labels in the particles can have an emission wavelength between about 400 nm and about 600 nm. In some embodiments, the detectable labels in the particles can have an emission wavelength of about 410 nm, about 420 nm, about 430 nm, about 440 nm, about 450 nm, about 460 nm, about 470 nm, about 480 nm, about 490 nm, about 510 nm, about 520 nm, about 530 nm, about 540 nm, about 550 nm, about 560 nm, about 570 nm, about 580 nm, or about 590 nm. In some embodiments, the detectable labels have a maximum emission wavelength of about 460 nm.

In some embodiments, the detectable labels in the particles can mimic a DNA stain, such as a fluorescent DNA stain. In some embodiments, the detectable labels in the particles are or comprise a blue fluorescent dye. In some embodiments, the detectable labels in the particles can mimic a nuclear stain such as DAPI.

In some embodiments, the particles are functionalized to attach to the solid support. In some embodiments, the beads in the particles are amine-modified. In some embodiments, the beads comprise latex beads. In some embodiments, the beads comprise hydrogel beads. In some embodiments, the beads are amine-modified polystyrene beads.

In some embodiments, the ratio between the number of the particles and the number of synthetic constructs is about 1:100 or lower. In some embodiments, the ratio between the number of the particles and the number of synthetic constructs is about 1:50 or lower. In some embodiments, the ratio between the number of the particles and the number of synthetic constructs is about 1:10 or lower. In some embodiments, the ratio between the number of the particles and the number of synthetic constructs is about 1.5 or lower. In some embodiments, the ratio between the number of the particles and the number of synthetic constructs is between about 1:5 to about 1:5000. In some embodiments, the ratio between the number of the particles and the number of synthetic constructs is between about 1:5 to about 1:500. In some embodiments, the ratio between the number of the particles and the number of synthetic constructs is between about 1:10 to about 1:50. In some embodiments, the ratio between the number of the particles and the number of synthetic constructs is about 1:5 or lower.

In some embodiments, the ratio between the number of the particles and the number of synthetic constructs is between about 1:5 and about 1:5,000. In some embodiments, the ratio between the number of the particles and the number of synthetic constructs is between about 1:5 and about 1:500. In some embodiments, the ratio between the number of the particles and the number of synthetic constructs is between about 1:5 and about 1:100.

In some embodiments, the solid support comprises a planar substrate. In some embodiments, the solid support comprises a glass or plastic substrate. In some embodiments, the plurality of synthetic constructs and/or the plurality of particles are immobilized in multiple regions on the solid support. In some embodiments, the multiple regions do not overlap with each other. In some embodiments, two or more of the multiple regions partially overlap with each other. In some embodiments, one or more of the multiple regions each independently comprises one or more fiducial markers. In some embodiments, two or more or all of the multiple regions are discrete regions. In some embodiments, the plurality of synthetic constructs and/or the plurality of particles are immobilized in two, three, four, or more discrete regions on the solid support.

In some embodiments, the plurality of synthetic constructs and/or the plurality of particles comprise a functional group configured to react with the functional groups of the solid support, e.g., to form covalent bonds. In some embodiments, the functional group of the plurality of synthetic constructs and/or the plurality of particles comprises amine and the functional groups of the solid support comprise an N-hydroxysuccinimide (NHS) moiety. For example, the solid support comprises an NHS ester reagent or a sulfo-NHS ester reagent that reacts with primary amines in the synthetic constructs and/or primary amines in the particles, each reaction forming a stable conjugate comprising an amide bond. The solid support can be NHS ester- or sulfo-NHS ester-activated prior to contacting the synthetic constructs and/or the particles.

A wide variety of different solid supports can be used, as long as the solid support is compatible with the synthetic constructs deposited thereon, probe hybridization, and signal detection (e.g., optical imaging such as fluorescence microscopy). A solid support can comprise any suitable support material and is generally transparent. In some embodiments, a glass slide is used. In some embodiments, a cover slip is used. The solid support can include, but are not limited to, glass, modified and/or functionalized glass, hydrogels, films, membranes, plastics, nylon, ceramics, resins, Zeonor, silica or silica-based materials including silicon and modified silicon, carbon, metals, inorganic glasses, optical fiber bundles, and polymers, such as polystyrene, cyclic olefin copolymers (COCs), cyclic olefin polymers (COPs), polypropylene, polyethylene and polycarbonate. The solid support can also correspond to a flow cell. In some embodiments, the solid support is between about 0.01 mm and about 5 mm, e.g., between about 0.05 mm and about 3 mm, between about 0.1 mm and about 2.5 mm, between about 0.2 mm and about 2 mm, between about 0.5 mm and about 1.5 mm, or about 1 mm in thickness. In some embodiments, the first substrate is or is about 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, or 2.0 mm in thickness, or of a thickness in between any one of the aforementioned values.

In some embodiments, the solid support for synthetic constructs for qualifying an instrument and the solid support for a biological sample (e.g., a cell or tissue sample) that is analyzed using the instrument for in situ analyte detection are the same or different. For instance, the solid support of an instrument QC slide can be of the same material(s) or different materials compared to the solid support of a cell or tissue sample slide.

(iii) Detectably Labeled Probes and Intermediate Probes

In some embodiments, the methods and kits herein comprise detectably labeled probes configured to hybridize to the identifier sequences in the synthetic constructs such as RCPs. In some embodiments, the methods and kit comprise intermediate probes configured to hybridize to the identifier sequences in the synthetic constructs, and detectably labeled probes configured to hybridize to at least some of the intermediate probes.

In some embodiments, each intermediate probe comprises i) a recognition sequence configured to hybridize to one of the identifier sequences, and ii) a hybridization sequence configured to hybridize to one of the detectably labeled probes. In some embodiments, the hybridization sequence is in a 3′ overhang of an intermediate probe. In some embodiments, the hybridization sequence is in a 5′ overhang of an intermediate probe. In some embodiments, the hybridization sequence is in a 3′ overhang and a 5′ overhang of an intermediate probe (e.g., the hybridization sequence is in a split region of the intermediate probe). In some embodiments, the detectably labeled probes are detected under excitation wavelengths of about 490 nm, about 530 nm, about 590 nm, or about 650 nm. In some embodiments, one or more of the identifier sequences can comprise a sequence of a gene or complement thereof.

In some embodiments, one or more of the identifier sequences can comprise a barcode sequence. In some embodiments, the barcode sequence is an artificial sequence that corresponds to a sequence of a gene or complement thereof. In some embodiments, the number of different identifier sequences in the synthetic constructs (e.g., RCPs) is at least 4, at least 6, at least 8, at least 10, at least 12, at least 14, at least 16, or more. In some embodiments, the number of different identifier sequences in the synthetic constructs is 9. In some embodiments, each different identifier sequence corresponds to a different reference gene. In some embodiments, a plurality of reference genes and identifier sequences associated therewith are used for qualifying an instrument. In some embodiments, the number of different detectably labeled probes in the kit is at most or about 3, at most or about 4, at most or about 5, at most or about 6, at most or about 7, or at most or about 8. In some embodiments, each different detectably labeled probe has a different nucleic acid sequence (e.g., one that hybridizes to the hybridization sequence of an intermediate probe disclosed herein) and a corresponding different detectable label, e.g., a fluorophore of a different color under a fluorescent microscope. In some embodiments, the number of different detectably labeled probes is 4 and the number of different identifier sequences in the synthetic constructs is 9 or more.

In some aspects, provided herein is a method of qualifying an instrument, comprising: a) placing a solid support on the instrument, wherein the instrument comprises: reagents comprising intermediate probes and fluorescently labeled probes, a fluidics module, an optics module, and a system controller, and wherein the solid support comprises rolling circle amplification products (RCPs) immobilized thereon, each RCP comprising multiple copies of an identifier sequence having an assigned signal code sequence from a codebook; b) in a first cycle, using the fluidics module to deliver to the solid support a first plurality of intermediate probe/fluorescently labeled probe pairs, wherein the intermediate probe and the fluorescently labeled probe in each pair form a complex comprising the intermediate probe hybridized to an RCP of the plurality of RCPs and the fluorescently labeled probe hybridized to the intermediate probe, wherein the intermediate probe comprises (i) a recognition sequence complementary to the identifier sequence in the RCP and (ii) an overhang sequence, and wherein the fluorescently labeled probe comprises (i) a sequence complementary to the overhang sequence and (ii) a fluorescent label; c) using the optics module to detect first signals (or absence thereof) associated with the fluorescent labels of the first plurality of probe pairs at multiple locations on the solid support, wherein the first signal or absence thereof detected at a particular location corresponds to a first signal code in the signal code sequence assigned to the identifier sequence in the RCP at the particular location; d) in a second cycle, using the fluidics module to deliver to the solid support a second plurality of intermediate probe/fluorescently labeled probe pairs, wherein the intermediate probe and the fluorescently labeled probe in each pair form a complex comprising the intermediate probe hybridized to an RCP of the plurality of RCPs and the fluorescently labeled probe hybridized to the intermediate probe, wherein the intermediate probe comprises (i) a recognition sequence complementary to the identifier sequence in the RCP and (ii) an overhang sequence, and wherein the fluorescently labeled probe comprises (i) a sequence complementary to the overhang sequence and (ii) a fluorescent label; e) using the optics module to detect second signals (or absence thereof) associated with the fluorescent labels of the second plurality of probe pairs at multiple locations on the solid support, wherein the second signal or absence thereof detected at a particular location corresponds to a second signal code in the signal code sequence assigned to the identifier sequence in the RCP at the particular location, thereby generating a signal code sequence comprising at least the first signal code and the second signal code at each of the multiple locations; f) using the system controller to compare the generated signal code sequences for the RCPs at the multiple locations to those from the codebook, thereby decoding the identifier sequences in the RCPs; and g) qualifying the instrument for detecting analytes in a biological sample based on the decoding in f). In some embodiments, the instrument is qualified using one or more pre-defined criteria described herein, for instance, if at least 90% of different identifier sequences in the RCPs are decoded to genes in a set of reference genes using the instrument, then the instrument is qualified for detecting analytes in situ in a biological sample.

In some embodiments, the synthetic constructs comprise sequences that do not hybridize to the detectably labeled probes or intermediate probes. In some embodiments, the synthetic constructs comprise sequences that are not detectable. In some embodiments, circular or circularized probes used for RCA comprise sequences that are not detectable. In some aspects, a single particle comprises synthetic constructs that are detectable and synthetic constructs that are not detectable (e.g., do not hybridize to the detectably labeled probes or intermediate probes). In some cases, the synthetic constructs that are not detectable (e.g., do not hybridize to the detectably labeled probes or intermediate probes) lowers density of signals detected at a particular location. In some embodiments, with the RCPs comprising identifier sequences, a plurality of additional RCPs that are not detectable using detectably labeled probes is tethered to the plurality of particles.

In some embodiments, in the first cycle, a first pool of intermediate probes and a universal pool of fluorescently labeled probes are delivered to the solid support, wherein each intermediate probe in the first pool of intermediate probes comprises (i) a recognition sequence complementary to one of the different identifier sequences in the RCPs, and (ii) a hybridization sequence complementary to a fluorescently labeled probe of the universal pool. In some embodiments, in the second cycle, a second pool of intermediate probes and the universal pool of fluorescently labeled probes are delivered to the solid support, wherein each intermediate probe in the second pool of intermediate probes comprises (i) a recognition sequence complementary to one of the different identifier sequences in the RCPs, and (ii) a hybridization sequence complementary to a fluorescently labeled probe of the universal pool. In some embodiments, the number of different identifier sequences in the RCPs is at least 9 and the number of fluorescently labeled probes of different sequences in the universal pool is 4.

In some embodiments, each fluorescently labeled probe of a different sequence in the universal pool is labeled with a fluorophore of a different color. In some embodiments, the signal code sequence comprising the first signal code, the second signal code, a third signal code corresponding to a third cycle, and a fourth signal code corresponding to a fourth cycle. In some embodiments, the signal code sequence can comprises a dark signal code corresponding to the absence of signal in the corresponding cycle.

The recognition sequences of the intermediate probes may be of any length. If more than one recognition sequence is used, the recognition sequences may independently have the same or different lengths. In some embodiments, the recognition sequence is at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the recognition sequence is no more than 48, no more than 40, no more than 32, no more than 24, no more than 16, no more than 12, no more than 10, no more than 8, or no more than 6 nucleotides in length. Combinations of any one of these are also possible, e.g., the recognition sequence may have a length of between 5 and 8, between 6 and 12, or between 7 and 15 nucleotides, etc. In one embodiment, the recognition sequence is of the same length as a barcode sequence or complement thereof in an RCP that the intermediate probe detects. In some embodiments, the recognition sequence is at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to the barcode sequence or complement thereof.

Likewise, the hybridization sequences of the intermediate probes may be of any length. If more than one hybridization sequence is used, the hybridization sequences may independently have the same or different lengths. In some embodiments, the hybridization sequence is at least 4, at least 5, least 6, least 7, least 8, least 9, at least 10, least 11, least 12, least 13, least 14, at least 15, least 16, least 17, least 18, least 19, at least 20, at least 25, at least 30, at least 35, at least 40, or at least 50 nucleotides in length. In some embodiments, the hybridization sequence is no more than 48, no more than 40, no more than 32, no more than 24, no more than 16, no more than 12, no more than 10, no more than 8, or no more than 6 nucleotides in length. Combinations of any one of these are also possible, e.g., the recognition sequence may have a length of between 5 and 8, between 6 and 12, or between 7 and 15 nucleotides, etc. In some embodiments, the hybridization sequence is at least 50%, at least 60%, at least 70%, at least 75%, at least 80%, at least 85%, at least 90%, at least 92%, at least 94%, at least 95%, at least 96%, at least 97%, at least 98%, at least 99%, or at least 100% complementary to a fluorescently labeled probe.

### B. Methods of Manufacturing QC Slides

In some embodiments, the methods provided herein comprise generating a synthetic construct disclosed herein. In some embodiments, a synthetic construct is an amplification product of a circular probe or a circularizable probe or probe set. In some embodiments, the method comprises circularizing a circularizable probe or probe set that hybridizes to a splint oligonucleotide in a solution. In some embodiments, the probe or probe set is circularizable by ligation using the splint oligonucleotide as a template.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any one of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9º N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, and the ends can be separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides is “filled” by one or more “gap” (oligo)nucleotide(s) which are complementary to a splint, circularizable probe or probe set (e.g., padlock probe), or target nucleic acid. The gap may be a gap of 1 to 60 nucleotides or a gap of 1 to 40 nucleotides or a gap of 3 to 40 nucleotides, or any integer (or range of integers) of nucleotides in between the indicated values. In some embodiments, the gap is of 1, 2, 3, 4, 5, 6, 7, 8, 9, or 10 or more nucleotides. In some embodiments, the gap between said terminal regions is filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

In some embodiments, a synthetic construct (e.g., RCP) is generated using a primer that is a single-stranded nucleic acid sequence having a 3′ end that is used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers are used to prime RNA synthesis and vice versa (e.g., RNA primers are used to prime DNA synthesis). Primers can vary in length. For example, primers are about 6 bases to about 120 bases. For example, primers can include up to about 25 bases. A primer, in some cases, refers to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, the method comprises performing amplification of circularizable probes or probe sets (e.g., following circularization of the probes or probe sets) outside a cell or tissue sample, e.g., in a solution. In some embodiments, the method comprises performing amplification of circular probes. In some embodiments, the amplification is performed at a temperature between or between about 20° C. and about 60° C. In some embodiments, the amplification is performed at a temperature between or between about 30° ° C. and about 40° C. In some aspects, the amplification step, such as the rolling circle amplification (RCA) is performed at a temperature between at or about 25° C. and at or about 50° C., such as at or about 25° C., 27° ° C., 29° C., 31ºC, 33ºC, 35° ° C., 37° C., 39ºC, 41ºC, 43° ° C., 45° C., 47ºC, or 49° C.

In some embodiments, upon addition of a DNA polymerase in the presence of appropriate dNTP precursors and other cofactors, a primer is elongated to produce multiple copies of the circular template (e.g., a circular or circularized probe or probe set). This amplification step can utilize isothermal amplification or non-isothermal amplification. In some embodiments, after the formation of the hybridization complex and association of the amplification probe, the hybridization complex is rolling-circle amplified to generate a cDNA nanoball containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 November 15; 49(11): 2540-2550; Schweitzer et al. Proc. Natl Acad. Sci. USA 97:101 13-119, 2000; Faruqi et al, BMC Genomics 2:4, 2000; Nallur et al, Nucl. Acids Res. 29:e118, 2001; Dean et al. Genome Res. 11:1095-1099, 2001; Schweitzer et al, Nature Biotech. 20:359-365, 2002; U.S. Pat. Nos. 6,054,274, 6,291,187, 6,323,009, 6,344,329 and 6,368,801). Examples of polymerases for use in RCA comprise DNA polymerase such phi29 (q29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics are employed. In some embodiments, the polymerase is phi29 DNA polymerase.

In some aspects, amplification of circular or circularized probes or probe sets is performed on a particle (e.g., a bead). The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be a solid particle (e.g., a metal-based particle including but not limited to iron oxide, gold or silver) covered with a coating comprising one or more polymers.

A gel bead may be a hydrogel bead. A gel bead may be formed from molecular precursors, such as a polymeric or monomeric species. A bead may be a semi-solid bead. In some embodiments, the bead may be a liposomal bead. A bead may be a solid bead. In some embodiments, the bead comprises metals including iron oxide, gold, and silver. In some cases, the bead is a silica bead. In some cases, the particle, e.g., a bead, is rigid. In other cases, the particle, e.g., a bead, is flexible and/or compressible. In some embodiments, the bead is a polyacrylamide bead.

In some cases, disulfide linkages are formed between molecular precursor units (e.g., monomers, oligomers, or linear polymers) or precursors incorporated into a bead and nucleic acid molecules (e.g., oligonucleotides). Cystamine (including modified cystamines), for example, is an organic agent comprising a disulfide bond that may be used as a crosslinker agent between individual monomeric or polymeric precursors of a bead. Polyacrylamide may be polymerized in the presence of cystamine or a species comprising cystamine (e.g., a modified cystamine) to generate polyacrylamide gel beads comprising disulfide linkages (e.g., chemically degradable beads comprising chemically-reducible cross-linkers). The disulfide linkages may permit the bead to be degraded (or dissolved) upon exposure of the bead to a reducing agent.

In some cases, chitosan, a linear polysaccharide polymer, is crosslinked with glutaraldehyde via hydrophilic chains to form a bead. Crosslinking of chitosan polymers may be achieved by chemical reactions that are initiated by heat, pressure, change in pH, and/or radiation.

In some cases, the particle (e.g., bead) comprises an acrydite moiety, which in certain aspects may be used to attach one or more nucleic acid molecules (e.g., an oligonucleotide comprising a primer sequence). In some embodiments, the particle (e.g., bead) is functionalized with an oligonucleotide (e.g., TTTTTTTTTTGGCTCCACTAAATAGACGCA, SEQ ID No: 1) comprising an acrydite moiety and/or an amine functional group. In some embodiments, the particle (e.g., bead) is functionalized with an oligonucleotide comprising an acrydite moiety and an amine functional group. In some embodiments, the particle (e.g., bead) is functionalized with an oligonucleotide comprising an acrydite moiety and an amine functional group coupled to a fluorescent dye. In some embodiments, the particle (e.g., bead) is functionalized with an oligonucleotide comprising an acrydite moiety and an amine functional group coupled to a blue fluorescent dye. In some embodiments, the substrate comprises functional groups comprise an N-hydroxysuccinimide (NHS) moiety that react with amines in the oligonucleotide (e.g., anchor oligonucleotide) attached to the particle. In some embodiments, the particle (e.g., bead) comprises an oligonucleotide labeled with a dye (e.g., a blue fluorescent dye such as DAPI), where the oligonucleotide bind either to an RCP or a common sequence on an anchor oligonucleotide.

In some cases, an acrydite moiety can refer to an acrydite analogue generated from the reaction of acrydite with one or more species, such as, the reaction of acrydite with other monomers and cross-linkers during a polymerization reaction. Acrydite moieties may be modified to form chemical bonds with a species to be attached, such as a nucleic acid molecule (e.g., an oligonucleotide). Acrydite moieties may be modified with thiol groups capable of forming a disulfide bond or may be modified with groups already comprising a disulfide bond. The thiol or disulfide (via disulfide exchange) may be used as an anchor point for a species to be attached or another part of the acrydite moiety may be used for attachment. In some cases, attachment is reversible, such that when the disulfide bond is broken (e.g., in the presence of a reducing agent), the attached species is released from the bead. In other cases, an acrydite moiety can comprise a reactive hydroxyl group that may be used for attachment.

Functionalization of beads for attachment of nucleic acid molecules (e.g., anchor oligonucleotides) may be achieved through a wide range of different approaches, including activation of chemical groups within a polymer, incorporation of active or activatable functional groups in the polymer structure, or attachment at the pre-polymer or monomer stage in bead production. In some embodiments, after bead generation, functionalization of the beads (e.g., functionalizing gel beads for attachment of nucleic acid molecules or other functional groups) uses one or more click reactions, and functional groups that participate in a click reaction comprise, but are not limited to, azido/alkynyl; azido/cyclooctynyl; tetrazine/dienophile; thiol/alkynyl; cyano/1,2-amino thiol; and, nitrone/cyclooctynyl.

For example, precursors (e.g., monomers, cross-linkers) that are polymerized to form a bead may comprise acrydite moieties, such that when a bead is generated, the bead also comprises acrydite moieties. The acrydite moieties can be attached to a nucleic acid molecule (e.g., anchor oligonucleotide) that comprises one or more functional sequences, such as a primer sequence (e.g., a nucleic acid primer sequence complementary to a sequence of a circular or circularized probe and/or for amplifying a target nucleic acid sequence) that is useful for incorporation into the bead. The one or more functional sequences may include sequences that are the same for all nucleic acid molecules coupled to a given bead and/or sequences that are different across all nucleic acid molecules coupled to the given bead. The nucleic acid molecule may be incorporated into the bead.

In some embodiments, a plurality of oligonucleotides (e.g., anchor oligonucleotides) attached to the particle each comprises a universal sequence used to capture a plurality of different circular or circularized probes. In some embodiments a plurality of different circular or circularized probes (e.g., corresponding to different reference genes) are mixed together prior to being captured on the particle. In some embodiments, a plurality of different circular or circularized probes is captured on a single particle. In some embodiments, a plurality of different RCPs comprising barcode sequences associated with two or more different reference genes is generated on a single particle. In some embodiments, a plurality of oligonucleotides (e.g., anchor oligonucleotides) attached to the particle each comprises a common sequence for binding a detectably labeled oligonucleotide (e.g., coupled to a blue dye). In some embodiments, a plurality of oligonucleotides are attached to the particle wherein subsets of the oligonucleotides comprise different functional sequences that are used to capture subsets of corresponding circular or circularized probes. In some embodiments, a plurality of particles includes a first population of particles for binding a first population of circular or circularized probes and a second population of particles for binding a second population of circular or circularized probe, wherein the first and second population of circular or circularized probes correspond to different genes or different groups of genes. In some embodiments, a synthetic construct (e.g., RCP) is generated using oligonucleotides attached to the particle as a primer. For example, oligonucleotides attached to the particle are single-stranded nucleic acid sequences having a 3′ end that is used as a substrate for a nucleic acid polymerase in a nucleic acid extension reaction.

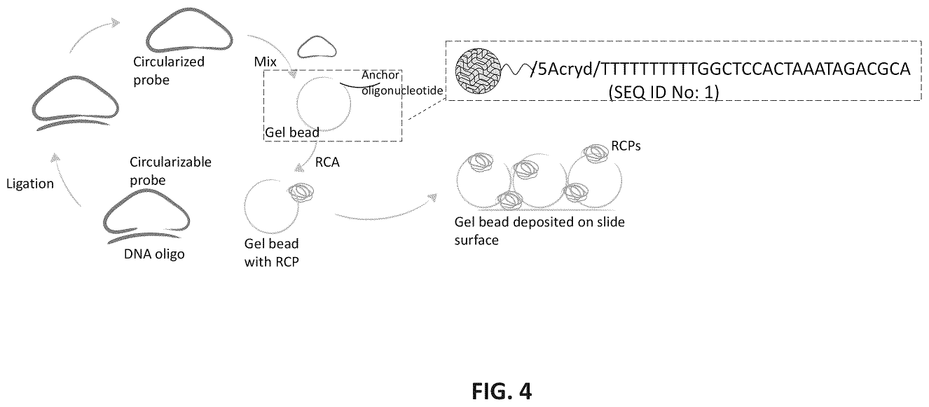

FIG. 4 illustrates an example of an oligonucleotide carrying particle (e.g., bead). In some embodiments, a nucleic acid molecule such as anchor oligonucleotide, is coupled to a functionalized bead. In some instances, a bead is coupled to one or more other nucleic acid molecules. In some instances, the oligonucleotide comprises a sequence that is used as a primer for downstream amplification reaction. In some embodiments, the oligonucleotides attached to a particle are used as a primer sequence to perform rolling circle amplification (optionally using a phi29 polymerase) of a template bound to the particle via the oligonucleotide. In some cases, the sequence of an oligonucleotide is bead-specific such that it is common to all nucleic acid molecules coupled to the same bead. In this case, a plurality of circular or circularized probes can bind to the same bead using the common sequence of the oligonucleotide. Alternatively or in addition, the bead may be coupled to a plurality of oligonucleotides comprising different sequences for binding different circular or circularized probes on the same bead to generate two or more different RCPs comprising different sequences for detection. In some embodiments, the sequence for binding a circular or circularized probe (e.g., anchoring sequence) is specific for each circular or circularized probe. In some embodiments, the oligonucleotide comprises an amplification primer sequence. In some aspects, ligation of the probes or probe sets is performed in solution prior to being contacted with a particle (e.g., a bead). In some aspects, ligation of the probes or probe sets is performed after being contacted with a particle (e.g., a bead). In some aspects, ligation of the probes or probe sets is performed in solution using a separate splint molecule to template the ligation. As illustrated, amplification of the circularized probes or probe sets is performed on the particle (e.g., a gel bead). After RCP generation, the gel beads with the attached generated RCPs are deposited on to the surface of a solid support (e.g., the slide surface).

In some embodiments, a plurality of particles comprises beads (e.g., gel beads) coupled to detectable labels and an oligonucleotide that comprises a sequence that is used as a primer for downstream amplification reaction. In some embodiments, a bead is coupled to detectable labels and a plurality of synthetic constructs (e.g., RCPs) generated on the bead. In some embodiments, the particle (e.g., bead) is functionalized with an oligonucleotide comprising an acrydite moiety and an amine functional group. In some embodiments, the particle (e.g., bead) is functionalized with an oligonucleotide comprising an acrydite moiety and an amine functional group. In some embodiments, the particle (e.g., bead) is coupled to a first plurality of oligonucleotides comprising a free 3′ end for extension (e.g., a primer oligonucleotide) and a second plurality of oligonucleotides conjugated to a fluorescent dye. In some embodiments, the ratio of first plurality of oligonucleotides to second plurality of oligonucleotides is tuned to achieve the desired concentration of RCPs and fluorescent dye-conjugated oligonucleotides attached to a single particle. In some embodiments, the second plurality of oligonucleotides comprise oligonucleotides conjugated to a fluorescent dye at the 3′ end. In some embodiments, the second plurality of oligonucleotides comprise oligonucleotides conjugated to a fluorescent dye at the 3′ end using an amine functional group. In some embodiments, the second plurality of oligonucleotides comprise oligonucleotides conjugated to a blue fluorescent dye at the 3′ end.

In some cases, precursors comprising a functional group that is reactive or capable of being activated such that it becomes reactive are polymerized with other precursors to generate gel beads comprising the activated or activatable functional group. The functional group may then be used to attach additional species (e.g., disulfide linkers, primers, other oligonucleotides, etc.) to the gel beads. For example, some precursors comprising a carboxylic acid (COOH) group can co-polymerize with other precursors to form a gel bead that also comprises a COOH functional group. In some cases, acrylic acid (a species comprising free COOH groups), acrylamide, and bis(acryloyl)cystamine are co-polymerized together to generate a gel bead comprising free COOH groups. The COOH groups of the gel bead can be activated (e.g., via 1-Ethyl-3-(3-dimethylaminopropyl)carbodiimide (EDC) and N-Hydroxysuccinimide (NHS) or 4-(4,6-Dimethoxy-1,3,5-triazin-2-yl)-4-methylmorpholinium chloride (DMTMM)) such that they are reactive (e.g., reactive to amine functional groups where EDC/NHS or DMTMM are used for activation). The activated COOH groups can then react with an appropriate species (e.g., a species comprising an amine functional group where the carboxylic acid groups are activated to be reactive with an amine functional group) comprising a moiety to be linked to the bead.

Beads comprising disulfide linkages in their polymeric network may be functionalized with additional species via reduction of some of the disulfide linkages to free thiols. The disulfide linkages may be reduced via, for example, the action of a reducing agent (e.g., DTT, TCEP, etc.) to generate free thiol groups, without dissolution of the bead. In some embodiments, the beads do not comprise disulfide linkages, Free thiols of the beads can then react with free thiols of a species or a species comprising another disulfide bond (e.g., via thiol-disulfide exchange) such that the species can be linked to the beads (e.g., via a generated disulfide bond). In some cases, free thiols of the beads may react with any other suitable group. For example, free thiols of the beads may react with species comprising an acrydite moiety. The free thiol groups of the beads can react with the acrydite via Michael addition chemistry, such that the species comprising the acrydite is linked to the bead. In some cases, uncontrolled reactions are prevented by inclusion of a thiol capping agent such as N-ethylmalieamide or iodoacetate.

Activation of disulfide linkages within a bead can be controlled such that only a small number of disulfide linkages are activated. Control may be exerted, for example, by controlling the concentration of a reducing agent used to generate free thiol groups and/or concentration of reagents used to form disulfide bonds in bead polymerization. In some cases, a low concentration (e.g., molecules of reducing agent: gel bead ratios of less than or equal to about 1:100,000,000,000, less than or equal to about 1:10,000,000,000, less than or equal to about 1:1,000,000,000, less than or equal to about 1:100,000,000, less than or equal to about 1:10,000,000, less than or equal to about 1:1,000,000, less than or equal to about 1:100,000, less than or equal to about 1:10,000) of reducing agent is used for reduction. Controlling the number of disulfide linkages that are reduced to free thiols may be useful in ensuring bead structural integrity during functionalization. In some cases, optically-active agents, such as fluorescent dyes, are coupled to beads via free thiol groups of the beads and used to quantify the number of free thiols present in a bead and/or track a bead.

In some embodiments, the particles are diluted to a desired concentration prior to being deposited on the support. In some aspects, particles coupled to a plurality of oligonucleotides may allow for a high density of RCPs to be attached. In some embodiments, the particles are deposited and uniformly spread evenly on the support. In some cases, different pools of particles are pooled and diluted to appropriate concentrations before being deposited on the support. In some embodiments, the particles functionalized with oligonucleotides bound to generated RCPs are mixed with spacer particles that are not functionalized with oligonucleotides bound to generated RCPs. In some examples, the spacer particles are functionalized with a functional group to attach to the solid support surface. In some case, the spacer particles comprise sodalime beads. In some cases, the spacer particles are functionalized with dipodal silane to attach to the solid support. In some cases, the ratio of spacer particle to particles functionalized with oligonucleotides for binding RCPs is tuned. In some aspects, spacer particles are used to deposit particles with tethered RCPs in a more uniform layer and/or to control boundaries of the deposited particles.

In some embodiments, the particles are sonicated after being diluted. In some embodiments, the solid support (e.g., slide) is cleaned before the particles are deposited on the surface. For example, the cleaning comprises ultra sonication in ethanol. In some embodiments, the slides are cleaned and dried before particles are deposited on the surface. In some embodiments, the particles are deposited on the surface in a buffer comprising a surfactant. In some cases, the surfactant is an anionic surfactant. In some embodiments, the particles are deposited on the surface in a buffer comprising sodium lauroyl sarcosinate (e.g., sarkosyl).

In some cases, addition of moieties to a particle (e.g., gel bead) after particle formation is advantageous. For example, addition of an oligonucleotide after gel bead formation may avoid loss of the species during chain transfer termination that can occur during polymerization. Moreover, smaller precursors (e.g., monomers or cross linkers that do not comprise side chain groups and linked moieties) may be used for polymerization and can be minimally hindered from growing chain ends due to viscous effects. In some cases, functionalization after gel bead synthesis can minimize exposure of species (e.g., oligonucleotides) to be loaded with potentially damaging agents (e.g., free radicals) and/or chemical environments. In some cases, the generated gel may possess an upper critical solution temperature (UCST) that can permit temperature driven swelling and collapse of a bead. Such functionality may aid in oligonucleotide (e.g., a primer oligonucleotide) infiltration into the bead during subsequent functionalization of the bead with the oligonucleotide. Post-production functionalization may also be useful in controlling loading ratios of species in beads, such that, for example, the variability in loading ratio is minimized. Species loading may also be performed in a batch process such that a plurality of beads can be functionalized with the species in a single batch.

In some embodiments, the generated and functionalized particles are contacted with a plurality of circular or circularized probes to generate RCPs on the particles. In some cases, the particles are functionalized and attached to a plurality of oligonucleotides (e.g., anchor oligonucleotides) comprising a sequence for binding to the circular or circularized probes. In some embodiments, the oligonucleotide is used as a primer to for an amplification reaction using the circular or circularized probe as template. In some embodiments, the RCPs generated on the particles are deposited on to a support (e.g., a substrate such as a slide). In some embodiments, the RCPs generated on the particles are deposited on to the surface of a solid support. In some cases, a plurality of particles with RCPs attached are deposited on the support. In some embodiments, a support with acrydite moieties is used and RCA is performed on the support. In some aspects, performing amplification of circular or circularized probes or probe sets is performed on a particle (e.g., a bead) before it is deposited on the solid support (e.g., in solution). In some aspects, performing amplification of circular or circularized probes or probe sets is performed on a particle (e.g., a bead) after it is deposited on the solid support.

In some embodiments, the RCPs are labeled with a detectably labeled oligonucleotide. In some embodiments, the RCPs are labeled with a detectably labeled oligonucleotide comprising a blue fluorescent dye. In some embodiments, a sequence of the RCPs is bound to a detectably labeled oligonucleotide comprising a blue fluorescent dye. In some embodiments, a sequence of the RCPs is bound to an oligonucleotide and the double stranded region is dyed with a blue fluorescent dye (e.g., DAPI). In some aspects, beads coupled to RCPs and a blue fluorescence dye mimics DAPI labeled nuclei in a cell with transcripts for detection. In some aspects, each single bead coupled to both RCPs and a blue fluorescence dye mimics a single cell with transcripts for detection. In some embodiments, labeling the same particles that are coupled to RCPs with a blue fluorescent dye can mimic cell nuclei and be used to assist in the decoding of the detect signals associated with the RCPs. In some embodiments, the blue fluorescent dye is used for cell segmentation. In some embodiments, cell segmentation is used for assigning detected signals and associated transcripts (e.g., RCPs are decoded to a gene in the set of reference genes) to cells (e.g., regions identified as cells). In some embodiments, cell segmentation and decoding results are used to cluster and/or compare cells (e.g., regions identified as cells).

In some embodiments, at least two different populations of particles are deposited on the solid support (e.g., slides). In some embodiments, the different populations of particles are mixed together and deposited on the solid support. In some cases, a plurality of particles is coupled to RCPs and an additional plurality of particles is coupled to a blue fluorescent dye. In some cases, the two different populations of particles are the same type of bead (e.g., gel beads). In some cases, the two different populations of particles are different types of bead. In some cases, the two different populations of particles are functionalized with different functional groups. In some cases, the two different populations of particles are functionalized with the same functional groups.

In some embodiments, a plurality of particles comprises beads (e.g., gel beads) directly coupled to detectable labels. In some embodiments, the detectable labels comprise a fluorescent dye. For example, a plurality of particles may comprise a blue fluorescent dye. In some embodiments, the particles are functionalized with a plurality of oligonucleotides conjugated to a fluorescent dye. For example, the particles comprise a plurality of oligonucleotides conjugated with blue fluorescent dyes. In some embodiments, the particles are conjugated to a plurality of oligonucleotides that are bound (e.g., hybridized) to detectably labeled oligonucleotides. For example, the particles comprise a plurality of oligonucleotides bound to detectably labeled oligonucleotides conjugated to a blue fluorescent dye. In some embodiments, the particles are conjugated to a plurality of anchor oligonucleotides that are bound (e.g., hybridized) to oligonucleotides and the double stranded region is dyed with a blue fluorescent dye (e.g., DAPI). In some embodiments, the particles comprise DAPI beads encapsulated in a hydrogel. In some embodiments, the particles labeled directly or indirectly with the fluorescent dye is different from the particles attached to a plurality of oligonucleotides comprising a sequence for binding to the circular or circularized probes and for generating RCPs. In some embodiments, the particles labeled directly or indirectly with the fluorescent dye are also attached to a plurality of oligonucleotides comprising a sequence for binding to the circular or circularized probes and for generating RCPs. In some embodiments, the particles are attached to a plurality of anchor oligonucleotides comprising a sequence for binding to the circular or circularized probes and for generating RCPs and the anchor oligonucleotide is bound to detectably labeled oligonucleotides conjugated to a blue fluorescent dye. In some embodiments, the particles are attached to a plurality of anchor oligonucleotides comprising a sequence for binding to the circular or circularized probes and for generating RCPs and the anchor oligonucleotide is bound to detectably labeled oligonucleotides, wherein the double stranded region is dyed with a blue fluorescent dye (e.g., DAPI). In some embodiments, the RCPs are tethered to the plurality of particles via an anchor oligonucleotide comprising a primer sequence previously used for generating the RCPs. In some embodiments, a plurality of particles are functionalized to attach two or more populations of oligonucleotides on each particle. For example, on a particle (e.g., gel bead), a first population of oligonucleotides are directly or indirectly labeled with a blue fluorescent dye and a second population of oligonucleotides comprise a primer sequence for generating RCPs. In some cases, on a particle (e.g., gel bead), the same population of oligonucleotides are used for multiple functions, e.g., to be labeled with a blue fluorescent dye and to be used as a primer sequence for generating RCPs.

In some embodiments, particles are deposited onto defined one or more regions of a support. In some embodiments, a plurality of beads with attached RCPs are deposited in two, three, four, or more discrete regions on the solid support, for instance, at the four corners of a surface area on the solid support. In some embodiments, particles are deposited onto a support using a nebulizer. In some embodiments, particles are deposited by placing a droplet of particles sandwiched between two slides. In some embodiments, a first slide with a functionalized surface facing up is contacted with a droplet of particles (e.g., particles with RCPs attached) and a second slide is placed with a functionalized surface facing down onto the droplet of particles. In some embodiments, the biological sample on the first substrate does not come into direct contact with the second substrate (e.g., slides) using one or more spacers. In some embodiments, the separation distance between first and second surface is maintained between 100 microns and 1 mm (e.g., between 200 microns and 800 microns, between 200 microns and 700 microns, between 200 microns and 600 microns, between 200 microns and 500 microns, between 200 microns and 400 microns, between 200 microns and 300 microns, between 400 microns and 600 microns), measured in a direction orthogonal to the surface of first or second slide. In some embodiments, the separation distance between first and second substrates is less than 1 mm. In some instances, the distance is 500 μm. In some instances, the distance is 2.5 microns. In some instances, the distance is about 200, 300, 400, 500, 600, 700 or 800 μm. In some embodiments, the two slides with the particles deposited in-between is allowed to dry completely. In some embodiments, the slide with particles deposited and dried is stored in a Mylar bag. In some cases the slides are stored to prevent exposure to light, moisture, and/or gas.

In some embodiments, the particle coated area, particle density on the support, and particle aggregation are evaluated after depositing the particles on the surface of the solid support. In some cases, the particles with RCPs attached are deposited on a substrate (e.g., a glass slide) and the particles are dissolvable after the RCPs are deposited. In some embodiments, once the particle is dissolved, the particles have deposited the RCPs in two-dimensional plane. In some cases, the solid support (e.g., slide) with deposited RCPs is dried before storage and/or use.

In some aspects, during the amplification step, modified nucleotides are added to the reaction to incorporate the modified nucleotides in the amplification product (e.g., nanoball). Examples of modified nucleotides comprise amine-modified nucleotides and/or nucleotides modified with a binder of a binding pair. The solid support can comprise a binding partner that binds to the binder (e.g., streptavidin or avidin that binds to a biotin moiety). In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification. In some embodiments, during the amplification step, the reaction comprises aminoallyl-dUTP and aminoallyl-dUTPs are incorporated in the amplification product (e.g., RCP). In some embodiments, the modified nucleotides (e.g., aminoallyl-dUTPs reacts with functional groups of the solid support (e.g., N-hydroxysuccinimide (NHS) moiety).

In some aspects, the polynucleotides and/or amplification product (e.g., amplicon) are anchored to a polymer matrix. For example, the polymer matrix can be a hydrogel. In some embodiments, the polymer matrix is a particle (e.g., a gel bead). In some embodiments, one or more of the polynucleotide probe(s) is/are modified to contain functional groups that can be used as an anchoring site to attach the polynucleotide probes and/or amplification product to a polymer matrix. Examples of modification and polymer matrix that can be employed in accordance with the provided embodiments comprise those described in, for example, US 2018/0051332, US 2019/0241950, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some aspects, provided herein is a method for producing a slide, comprising: a) separately generating rolling circle amplification products (RCPs) of each circular template of a plurality of different circular templates, wherein the RCPs of each circular template comprise multiple copies of a different identifier sequence which is assigned a different signal code sequence from a codebook; b) pooling RCPs of the plurality of different circular templates; c) disposing the pooled RCPs on a solid support, thereby producing the slide comprising the solid support and the pooled RCPs thereon. In some embodiments, the method comprises disposing a plurality of particles on the solid support prior to, concurrently with, or after disposing the pooled RCPs on the solid support. In some embodiments, the particles comprise latex beads coupled to a blue fluorescence dye (e.g., that mimics DAPI labeled nuclei in a cell). In some embodiments, the particles comprise hydrogel beads comprising a blue fluorescence dye.

In some embodiments, the RCPs of each circular template are generated outside a cell or tissue sample, generated in solution, and/or generated in vitro. Any one of more of the circular templates can be amplified using rolling circle amplification in a solution and outside a cell or tissue sample. In some embodiments, each one of a plurality of different circular templates is amplified in a separate solution, and the separate solutions are pooled. In some embodiments, each separate solution is separately concentrated (e.g, using a vacuum such as SpeedVac™), dried down, and/or lyophilized and then combined. In some embodiments, each one of the plurality of different circular templates is amplified separately to prevent RCPs comprising different identifier sequences (e.g., barcode sequences) from intertwining with one another, thereby facilitating decoding of different RCPs after they are combined and deposited on a solid support. In some embodiments, the method comprises providing or generating each circular template separately in a solution. In some embodiments, the circular template is generated by hybridizing a circularizable probe or probe set to a splint oligonucleotide, and circularizing the circularizable probe or probe set using the splint oligonucleotide as a template. In some embodiments, the circularizing comprises ligation templated on the splint oligonucleotide, with or without gap filling prior to the ligation. In some embodiments, the solution comprising the circular template is diluted prior to rolling circle amplification of the circular template. In some embodiments, the circular template is generated using non-templated ligation, such as click chemistry ligation or enzymatic ligation, e.g., using a ssDNA ligase such as a CircLigase™.

In some embodiments, the RCPs of each circular template can comprise modified nucleic acid residues. In some embodiments, one or more amine-modified nucleic acid residues are incorporated during rolling circle amplification.

In some embodiments, the solid support can comprise functional groups configured to bind to or react with one or more modified nucleic acid residues, thereby immobilizing RCPs comprising modified nucleic acid residues on the solid support. In some embodiments, the functional groups comprise an N-hydroxysuccinimide (NHS) moiety configured to react with amines in the RCPs and/or in the particles.

In some embodiments, the plurality of synthetic constructs (e.g., RCPs) and/or the plurality of particles are immobilized on the solid support via biotin-streptavidin affinity binding. For instance, the RCPs can be labeled with biotin or a derivative or analog thereof, e.g., by incorporating biotin-labeled nucleotides during RCA, and the biotin-labeled RCPs can be bound by streptavidin or avidin on a surface region of a solid support.

In some embodiments, the concentration of the plurality of synthetic constructs (e.g., RCPs) is controlled by titrating the concentration of circular probes or circularized probes used to perform the RCA. In some embodiments, the probes are hybridized to an oligonucleotide conjugated to a particle (e.g., gel bead). In some embodiments, a concentration of about 1 pM, about 5 pM, about 10 pM, about 20 pM, about 30 pM, about 40 pM, about 50 pM, about 60 pM, about 70 pM, about 80 pM, about 90 pM, or about 100 pM of circular probes or circularized probes is used to perform RCA. In some embodiments, a concentration of less than or equal to about 1 pM, less than or equal to about 5 pM, less than or equal to about 10 pM, less than or equal to about 20 pM, less than or equal to about 30 pM, less than or equal to about 40 pM, less than or equal to about 50 pM, less than or equal to about 60 pM, less than or equal to about 70 pM, less than or equal to about 80 pM, less than or equal to about 90 pM, or less than or equal to about 100 pM of circular probes or circularized probes is used to perform RCA. In some embodiments, a concentration of between 1 pM-100 pM, between 1 pM-50 pM, between 1 pM-40 pM, between 1 pM-20 pM, between 1 pM-10 pM, between 1 pM-5 pM, between 10 pM-100 pM, between 10 pM-50 pM, between 10 pM-40 pM, between 10 pM-20 pM, between 5 pM-100 pM, between 5 pM-50 pM, between 5 pM-40 pM, between 5 pM-30 pM, between 5 pM-20 pM, between 5 pM-10 pM, between 20 pM-100 pM, between 20 pM-50 pM, or between 30 pM-50 pM of circular probes or circularized probes is used to perform RCA. In some embodiments, a concentration of about 20 pM of circular probes or circularized probes is used to perform RCA, wherein the circular probe or circularized probe is hybridized to an oligonucleotide conjugated to a particle (e.g., gel bead). In some embodiments, the concentration of the circular probes or circularized probes captured on a particle is titrated such that some proportion of oligonucleotides on a particle is not hybridized to a circular probe or circularized probe.

In some embodiments, the ratio between the number of the particles and the number of the RCPs is between about 1:5 and about 1:5,000. In some embodiments, the ratio between the number of the particles and the number of the RCPs is about 1:30. In some embodiments, the ratio between the number of the particles and the number of the RCPs is about 1:1,000.

In some embodiments, the RCPs are generated in solution or tethered to a particle (e.g., gel bead). In some embodiments, the RCPs are pooled outside a cell or tissue. Any two or more of the RCPs can be pooled in solution. Any two or more of the RCPs can be pooled in lyophilized form. In some embodiments, the pooled RCPs are not in a cell or tissue. In some embodiments, the RCPs are pooled and deposited on a solid support that is free of cells. In some embodiments, the pooled and deposited RCPs are not in a cell or tissue.

The pooled RCPs can be deposited in any one or more regions at any location on the solid support, for instance, at or near a corner or at or near the center of the solid support. In some embodiments, the pooled RCPs are deposited in two, three, four, or more discrete regions on the solid support, for instance, at the four corners of a surface area on the solid support. In some embodiments, the solid support can comprise one or more fiducial markers. In some embodiments, the pooled RCPs are deposited evenly across the surface of a solid support.

### III. Instruments for Analysis of Biological Samples

Target molecules (e.g., nucleic acids, proteins, antibodies, etc.) can be detected in biological samples (e.g., one or more cells or a tissue sample) using an instrument having integrated optics and fluidics modules (an “opto-fluidic instrument” or “opto-fluidic system”). In an opto-fluidic instrument, the fluidics module is configured to deliver one or more reagents (e.g., fluorescent probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more probing cycles. In various embodiments, the captured images are processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (e.g., in situ) within the biological sample. For example, an opto-fluidic instrument may be an in situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection via probe hybridization, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes within the sample) and/or imaging light signals received from the probed sample. The in situ analysis system may also include other ancillary modules configured to facilitate the operation of the opto-fluidic instrument, such as, but not limited to, cooling systems, motion calibration systems, etc.

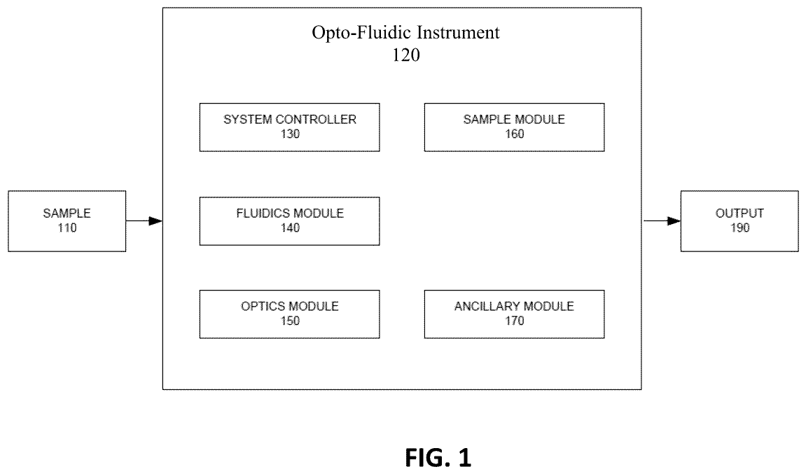

FIG. 1 shows an example workflow of analysis of a biological sample 110 (e.g., cell or tissue sample) using an opto-fluidic instrument 120, according to various embodiments. In various embodiments, the sample 110 is a biological sample (e.g., a tissue) that includes molecules such as DNA, RNA, proteins, antibodies, etc. For example, the sample 110 can be a sectioned tissue that is treated to access the RNA thereof for labeling with circularizable DNA probes. Ligation of the probes may generate a circular DNA probe which can be enzymatically amplified and bound with fluorescent oligonucleotides, which can create bright signal that is convenient to image and has a high signal-to-noise ratio.

In various embodiments, the sample 110 is placed in the opto-fluidic instrument 120 for analysis and detection of the molecules in the sample 110. In various embodiments, the opto-fluidic instrument 120 is a system configured to facilitate the experimental conditions conducive for the detection of the target molecules. For example, the opto-fluidic instrument 120 can include a fluidics module 140, an optics module 150, a sample module 160, and an ancillary module 170, and these modules may be operated by a system controller 130 to create the experimental conditions for the probing of the molecules in the sample 110 by selected probes (e.g., circularizable DNA probes), as well as to facilitate the imaging of the probed sample (e.g., by an imaging system of the optics module 150). In various embodiments, the various modules of the opto-fluidic instrument 120 are separate components in communication with each other, or at least some of them are integrated together.

In various embodiments, the sample module 160 is configured to receive the sample 110 into the opto-fluidic instrument 120. For instance, the sample module 160 may include a sample interface module (SIM) that is configured to receive a sample device (e.g., cassette) onto which the sample 110 can be deposited. That is, the sample 110 may be placed in the opto-fluidic instrument 120 by depositing the sample 110 (e.g., the sectioned tissue) on a sample device that is then inserted into the SIM of the sample module 160. In some instances, the sample module 160 may also include an X-Y stage onto which the SIM is mounted. The X-Y stage may be configured to move the SIM mounted thereon (e.g., and as such the sample device containing the sample 110 inserted therein) in perpendicular directions along the two-dimensional (2D) plane of the opto-fluidic instrument 120. Additional discussion related the SIM can be found in U.S. Provisional Application No. 63/348,879, filed Jun. 3, 2022, titled “Methods, Systems, and Devices for Sample Interface,” which is incorporated herein by reference in its entirety.

The experimental conditions that are conducive for the detection of the molecules in the sample 110 may depend on the target molecule detection technique that is employed by the opto-fluidic instrument 120. For example, in various embodiments, the opto-fluidic instrument 120 is a system that is configured to detect molecules in the sample 110 via hybridization of probes. In such cases, the experimental conditions can include molecule hybridization conditions that result in the intensity of hybridization of the target molecule (e.g., nucleic acid) to a probe (e.g., oligonucleotide) being significantly higher when the probe sequence is complementary to the target molecule than when there is a single-base mismatch. The hybridization conditions include the preparation of the sample 110 using reagents such as washing/stripping reagents, hybridizing reagents, etc., and such reagents may be provided by the fluidics module 140.

In various embodiments, the fluidics module 140 may include one or more components that are used for storing the reagents, as well as for transporting said reagents to and from the sample device containing the sample 110. For example, the fluidics module 140 may include reservoirs configured to store the reagents, as well as a waste container configured for collecting the reagents (e.g., and other waste) after use by the opto-fluidic instrument 120 to analyze and detect the molecules of the sample 110. Further, the fluidics module 140 may also include pumps, tubes, pipettes, etc., that are configured to facilitate the transport of the reagent to the sample device (e.g., and as such the sample 110). For instance, the fluidics module 140 may include pumps (“reagent pumps”) that are configured to pump washing/stripping reagents to the sample device for use in washing/stripping the sample 110 (e.g., as well as other washing functions such as washing an objective lens of the imaging system of the optics module 150).

In various embodiments, the ancillary module 170 is a cooling system of the opto-fluidic instrument 120, and the cooling system may include a network of coolant-carrying tubes that are configured to transport coolants to various modules of the opto-fluidic instrument 120 for regulating the temperatures thereof. In such cases, the fluidics module 140 may include coolant reservoirs for storing the coolants and pumps (e.g., “coolant pumps”) for generating a pressure differential, thereby forcing the coolants to flow from the reservoirs to the various modules of the opto-fluidic instrument 120 via the coolant-carrying tubes. In some instances, the fluidics module 140 comprises one or more returning coolant reservoirs that are configured to receive and store returning coolants, e.g., heated coolants flowing back into the returning coolant reservoirs after absorbing heat discharged by the various modules of the opto-fluidic instrument 120. In such cases, the fluidics module 140 may also include cooling fans that are configured to force air (e.g., cool and/or ambient air) into the returning coolant reservoirs to cool the heated coolants stored therein. In some instance, the fluidics module 140 may also include cooling fans that are configured to force air directly into a component of the opto-fluidic instrument 120 so as to cool said component. For example, the fluidics module 140 may include cooling fans that are configured to direct cool or ambient air into the system controller 130 to cool the same.

As discussed above, the opto-fluidic instrument 120 may include an optics module 150 which include the various optical components of the opto-fluidic instrument 120, such as but not limited to a camera, an illumination module (e.g., LEDs), an objective lens, and/or the like. The optics module 150 may include a fluorescence imaging system that is configured to image the fluorescence emitted by the probes (e.g., oligonucleotides) in the sample 110 after the probes are excited by light from the illumination module of the optics module 150.

In some instances, the optics module 150 comprises an optical frame onto which the camera, the illumination module, and/or the X-Y stage of the sample module 160 is mounted.

In various embodiments, the system controller 130 is configured to control the operations of the opto-fluidic instrument 120 (e.g., and the operations of one or more modules thereof). In some instances, the system controller 130 may take various forms, including a processor, a single computer (or computer system), or multiple computers in communication with each other. In various embodiments, the system controller 130 is communicatively coupled with data storage, set of input devices, display system, or a combination thereof. In some cases, some or all of these components are considered to be part of or otherwise integrated with the system controller 130, are separate components in communication with each other, or are integrated together. In other examples, the system controller 130 is, or is in communication with, a cloud computing platform. In some embodiments, the opto-fluidic instrument is coupled to or configured to be coupled to the cloud (e.g., a cloud computing platform) via a network.

In various embodiments, the opto-fluidic instrument 120 may analyze the sample 110 and may generate the output 190 that includes indications of the presence of the target molecules in the sample 110. For instance, with respect to the example embodiment discussed above where the opto-fluidic instrument 120 employs a hybridization technique for detecting molecules, the opto-fluidic instrument 120 may cause the sample 110 to undergo successive rounds of fluorescent probe hybridization (using two or more sets of fluorescent probes, where each set of fluorescent probes is excited by a different color channel) and be imaged to detect target molecules in the probed sample 110. In such cases, the output 190 may include optical signatures (e.g., a codeword) specific to each gene, which allow the identification of the target molecules.

In some aspects, the opto-fluidic instrument is used for analyzing, e.g., detecting or determining, one or more sequences present in a probe or product thereof, such as identifier sequences (e.g., barcode sequences) in nucleic acid molecules. For an instrument performance test disclosed herein (e.g., in Section IV), the nucleic acid molecules can be synthetic constructs (e.g., RCPs) generated in vitro (e.g., in a solution and outside a cell or tissue sample). For an in situ assay disclosed herein (e.g., in Section V), the nucleic acid molecules (e.g., RCPs) can be can generated in situ in a cell or tissue sample, such as an intact tissue section or a matrix-embedded and cleared biological sample. In some cases, the analysis is performed on one or more images captured, and may comprise processing the image(s) and/or quantifying signals observed. For example, the analysis may comprise processing information of one or more cell types, one or more types of analytes, a number or level of an analytes, and/or a number or level of cells and/or analytes detected in a particular region of the sample. In some embodiments, the analysis comprises detecting a sequence e.g., a barcode present in the sample. In some embodiments, the analysis includes quantification of puncta (e.g., if amplification products are detected). In some embodiments, the obtained information is compared to a positive and negative control, or to a threshold of a feature to determine if the sample exhibits a certain feature or phenotype. In some cases, the information comprises signals from a cell, a region, and/or comprise readouts from multiple detectable labels. In some case, the analysis further includes displaying the information from the analysis or detection step. In some embodiments, software is used to automate the processing, analysis, and/or display of data.

In some aspects, the results from an assay performed using an opto-fluidic instrument are analyzed as described in Section IV for assessment of the assay, for example, for assessing instrument performance. In some embodiments, once the opto-fluidic instrument passes the performance test, it is used for an in situ assay (e.g., disclosed herein in Section V).

### IV. Qualifying an Instrument for In Situ Assays

In some aspects, provided herein is a method for qualifying an instrument, comprising: a) placing a solid support on the instrument, wherein the instrument comprises: reagents comprising fluorescently labeled probes, a fluidics module, an optics module, and a system controller, and wherein the solid support comprises a plurality of synthetic constructs (e.g., RCPs) deposited thereon, each synthetic construct comprising an identifier sequence (e.g., multiple copies of an identifier sequence such as barcode sequences) having an assigned signal code sequence from a codebook; b) using the fluidics module to deliver, in sequential cycles, the fluorescently labeled probes to the solid support; c) using the optics module to detect, in the sequential cycles, signals (or absence thereof) associated with the fluorescently labeled probes directly or indirectly bound to the identifier sequences in the synthetic constructs, thereby generating signal code sequences for the synthetic constructs; d) using the system controller to compare the generated signal code sequences to those from the codebook, thereby decoding the identifier sequences in the synthetic constructs; and e) qualifying the instrument, wherein the instrument is suitable for detecting analytes in a biological sample when the decoding in d) meets one or more pre-defined criteria. In some aspects, provided herein is a method for qualifying an instrument workflow, including operations of the various components of the instrument and reagents on the instrument.

In some embodiments, the synthetic constructs comprise RCPs which can be decoded using the method by decoding the corresponding identifier sequences (e.g., gene sequences or barcode sequences) in the RCPs. In some embodiments, the identifier sequences in the RCPs can comprise sequences of a set of reference genes or complements thereof. In some embodiments, the identifier sequences in the RCPs can comprise barcode sequences corresponding to a set of reference genes or complements thereof. In some embodiments, the codebook can comprise signal code sequences each corresponding to a reference gene of the set of reference genes. In some embodiments, the codebook comprise signal code sequence that are sequences of color codes, arranged in the order of the corresponding signal color detected in sequential cycles of probe hybridization and imaging.

In some embodiments, detection of the identifier sequences (e.g., barcode sequences) in the synthetic constructs (e.g., RCPs generated in vitro) is performed by sequential hybridization of probes to the synthetic constructs and detecting complexes formed by the probes and identifier sequences. In some cases, each identifier sequence (e.g., barcode sequence) is assigned a sequence of signal codes that identifies it (e.g., a temporal signal signature or code that identifies the identifier sequence which in turn identifies the reference gene used for QC), and detecting the identifier sequences in the synthetic constructs can comprise decoding the identifier sequences by detecting the corresponding sequences of signal codes detected from sequential hybridization, detection, and removal of sequential pools of intermediate probes and the universal pool of detectably labeled probes. In some cases, the sequences of signal codes are sequences of fluorescent signals assigned to the corresponding identifier sequences. In some embodiments, the detectably labeled probes are fluorescently labeled. In some embodiments, decoding the identifier sequences (e.g., barcode sequences) is performed by sequential probe hybridization as described in US 2021/0340618, the content of which is herein incorporated by reference in its entirety.

In some embodiments, the detecting step can comprise contacting the QC slide with one or more detectably labeled probes that directly or indirectly hybridize to the identifier sequences (e.g., barcode sequences) in the synthetic constructs (e.g., RCPs generated in vitro), and dehybridizing the one or more detectably labeled probes. In some embodiments, the contacting and dehybridizing steps can be repeated with the one or more detectably labeled probes and/or one or more other detectably labeled probes that directly or indirectly hybridize to the identifier sequences. In some aspects, the method comprises sequential hybridization of detectably labeled probes to create a spatiotemporal signal signature or code that identifies reference gene used for QC.

In some embodiments, the detecting step can comprise contacting the QC slide with one or more first detectably labeled probes that directly hybridize to the identifier sequences (e.g., barcode sequences in RCPs). In some instances, the detecting step can comprise contacting the QC slide with one or more first detectably labeled probes that indirectly hybridize to the identifier sequences (e.g., barcode sequences in RCPs). In some embodiments, the detecting step can comprise contacting the QC slide with one or more second detectably labeled probes that directly or indirectly hybridize to the identifier sequences (e.g., barcode sequences in RCPs).

In some embodiments, the detecting step can comprise contacting the QC slide with one or more intermediate probes that directly or indirectly hybridize to the barcode sequences in the RCPs on the QC slide, wherein the one or more intermediate probes are detectable using one or more detectably labeled probes. In some embodiments, the detecting step can further comprise dehybridizing the one or more intermediate probes and/or the one or more detectably labeled probes from the barcode sequences in the RCPs on the QC slide. In some embodiments, the contacting and dehybridizing steps can be repeated with the one or more intermediate probes, the one or more detectably labeled probes, one or more other intermediate probes, and/or one or more other detectably labeled probes.

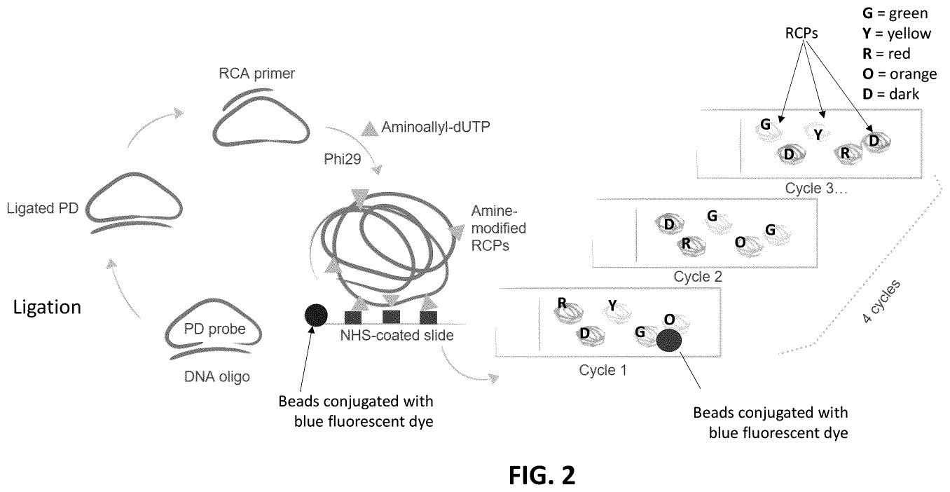

In some embodiments, for instance as shown in FIG. 2, by sequentially cycling detectably labeled probes on a QC slide, a sequence of color codes can be detected at a location in the QC slide by tracking the signal colors at the location through the sequential cycles. FIG. 2 depicts color codes associated with synthetic constructs (e.g., labeled RCPs) detected in each of cycles 1-3. The detected sequence of color codes can be compared to signal code sequences in the codebook, thereby decoding the sequence of color codes and mapping the location (and the RCP at the location) to a reference gene for the instrument performance test.

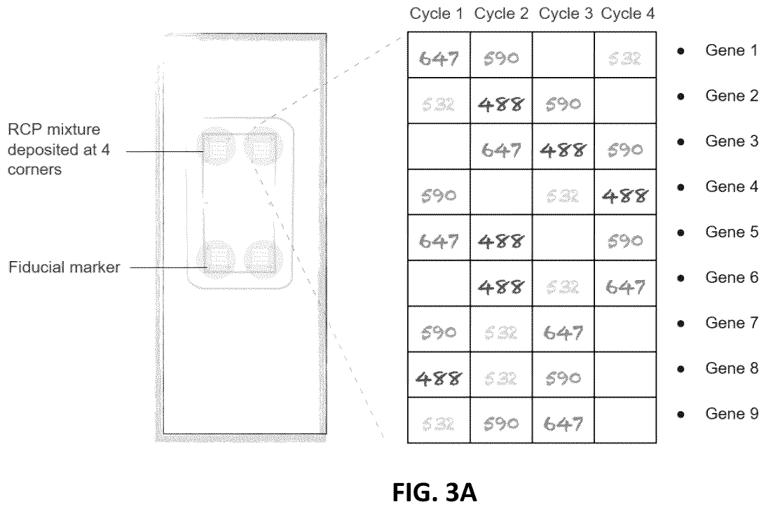

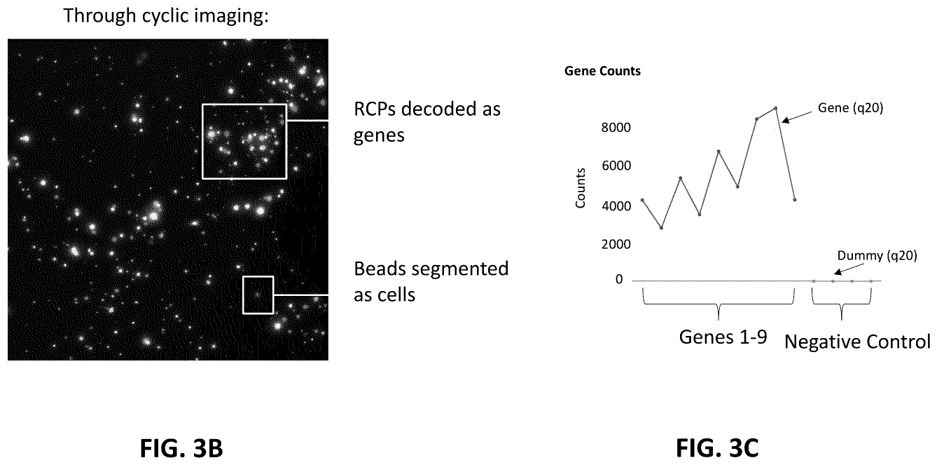

In some embodiments, the solid support can comprise a plurality of particles deposited thereon. In some embodiments, the plurality of particles are coupled to a fluorescent moiety. In some embodiments, the fluorescent moiety has a maximum excitation wavelength of about 350 nm. In some embodiments, the fluorescent moiety comprises a blue fluorescent dye. In some embodiments, the particles are beads coupled to a blue fluorescent dye. In some embodiments, the RCPs and the plurality of particles are deposited in two, three, four, or more discrete regions on the solid support, e.g., as shown in FIG. 3A. In some embodiments, the solid support may but does not need to comprise a cell or tissue sample deposited thereon. In some embodiments, the surface of the solid support is coated with particles and/or RCPs. For example, the area of the surface that can be imaged is coated with particles and/or RCPs. In some aspects, a plurality of FOVs is analyzed. For example, signals (e.g., associated with beads and synthetic constructs described in Section II) and in two or more discrete FOVs are detected and/or analyzed. For instance, any one or more of the FOVs selected can be at or near a corner of the solid support, such as at or near a corner of the whole imageable surface of a slide coated with particles and/or RCPs.

In some embodiments, the reagents for use on the instrument can comprise intermediate probes configured to hybridize to the identifier sequences in the RCPs, and the fluorescently labeled probes are configured to hybridize to the intermediate probes. As shown in FIG. 3A, in each cycle, a pair of an intermediate probe and a fluorescently labeled probe (that hybridizes to the intermediate probe of the pair) can be used to detect an identifier sequence (e.g., a barcode sequence) for one of a set of reference genes. Multiple pairs of intermediate probes and fluorescently labeled probes can be included in probes that are delivered to one or more regions on the QC slide, each pair targeting a different identifier sequence and the corresponding reference gene. In some embodiments, the number of different identifier sequences in the RCPs is at least 9, the number of fluorescently labeled probes of different sequences is 4, and the number of the sequential cycles is 4. In some embodiments, hybridizing probes to the identifier sequences in the RCPs comprises 4 sequential cycles. An example of a codebook is shown in FIG. 3A, where the sequence of color codes (or absence thereof) can be assigned to each of the nine reference genes. Based on the signals detected at various locations through Cycles 1-4, a sequence of color codes (or absence thereof) can be detected at each of the locations, indicating there is an RCP having a barcode sequence that corresponds to the detected sequence of color codes (or absence thereof). The detected sequences of color codes (or absence thereof) at the various locations can be compared to those in the codebook to decode whether the RCPs correspond to the reference genes, and if so, which RCP corresponds to which reference gene. The particles can mimic cell nuclei and be used to assist in the decoding, as shown in FIGS. 3B-3C.

In some embodiments, the reference genes comprise ALDHIA2, AKT1, BEX3, CDKN2A, CD3D, CD3E, CEBPE, DUXAP8, ECI1, ELL3, ENO2, FYB1, GSTM3, GSTP1, GTSF1, LAPTM4A, LEF1, MED28, MTAP, POLR2A, POU2AF1, RABIB, RFC2, SIX6, SLC39A3, SNHG32 and SPIB. In some embodiments, the reference genes comprise POLR2A, DUXAP8, RFC2, CD3D, FYB1, MTAP, AKT1, CDKN2A, and SPIB. In some aspects, provided herein are quality control slides comprising a plurality of RCPs associated with one or more of the following genes: ALDH1A2, AKT1, BEX3, CDKN2A, CD3D, CD3E, CEBPE, DUXAP8, ECI1, ELL3, ENO2, FYB1, GSTM3, GSTP1, GTSF1, LAPTM4A, LEF1, MED28, MTAP, POLR2A, POU2AF1, RAB1B, RFC2, SIX6, SLC39A3, SNHG32 and SPIB. In some aspects, provided herein are quality control slides comprising a plurality of RCPs associated with one or more of the following genes: POLR2A, DUXAP8, RFC2, CD3D, FYB1, MTAP, AKT1, CDKN2A, and SPIB. In some aspects, provided herein are quality control slides comprising a plurality of RCPs associated with a panel of genes comprising POLR2A, DUXAP8, RFC2, CD3D, FYB1, MTAP, AKT1, CDKN2A, and SPIB.

In some embodiments, the sizes and/or shapes of the particles are comparable to nuclei in a cell or tissue sample. The cell or tissue sample can be analyzed using the qualified instrument or can be a reference sample that is not analyzed using the qualified instrument. In some embodiments, the mean diameter of the particles is about 2 μm. In some embodiments, any one or more of the particles can be of a shape mimicking a cell, such as a round or oval shape. In some embodiments, the signal intensity of an individual particle (e.g., which functions as an artificial nucleus of a virtual cell for qualifying an instrument) on the solid support can be comparable to the signal intensity of a nucleus detected in the biological sample (e.g., a cell or tissue sample). In some embodiments, the ratio between the number of the particles and the number of the RCPs can be between about 1:5 and about 1:5,000.

In some embodiments, the instrument can comprise a sample module configured to receive and/or secure the solid support for QC (e.g., comprising particles and synthetic constructs such as RCPs for decoding using the instrument) and/or the biological sample for analyte detection after the QC. In some embodiments, the instrument can comprise an ancillary module configured to facilitate operation of the instrument. In some embodiments, the ancillary module comprises a cooling system and/or a motion calibration system. In some embodiments, the system controller can control operation of the fluidics module, the optics module, the sample module, and/or the ancillary module. In some embodiments, the system controller can comprise a processor, a computer, and/or a computing platform. In some embodiments, the processor, the computer, and/or the computing platform are integrated. In some embodiments, the processor, the computer, and/or the computing platform can comprise separate components configured to communicate with one another via a network. In some embodiments, the system controller can comprise or be configured to communicate with a cloud computing platform. In some embodiments, the system controller is communicatively coupled with a data storage, an input device, a display system, or a combination thereof.

In some embodiments, the biological sample for analysis using the instrument after QC disclosed herein is a cell or tissue sample. In some embodiments, the biological sample is a tissue section. In some embodiments, the biological sample is a matrix-embedded biological sample. In some embodiments, the biological sample is a cleared biological sample.

In some embodiments, the one or more pre-defined criteria can comprise that at least or about 80%, at least or about 85%, at least or about 90%, at least or about 95%, at least or about 99%, or all of the different identifier sequences in the in vitro synthetic RCPs are decoded using the instrument. In some embodiments, the one or more pre-defined criteria can comprise that at least 90% or at least 95% of different identifier sequences in the in vitro synthetic RCPs are successfully decoded using the instrument. In some embodiments, at least 90% of different identifier sequences in the in vitro synthetic RCPs are correctly decoded using the instrument, followed by using the instrument to detect analytes in situ in the biological sample. In some embodiments, each of the different identifier sequences is a barcode sequence that corresponds to a different gene in the set of reference genes used for instrument performance QC.

In some embodiments, if at least at least or about 80%, at least or about 85%, at least or about 90%, at least or about 95%, at least or about 99% of the set of reference genes in the RCPs on the QC slide are decoded using the instrument, the instrument is suitable for analyte detection in situ in a biological sample.

In some aspects, the method comprises determining a calculated score for an instrument performance metric, and in some cases, the score can be a quality score. In some aspects, the quality score is a Phred quality score (Q-score). For example, the quality score can be an indication of the quality of the barcode sequence(s) decoded during the assay. In some aspects, the quality score is an indication of the likelihood that the barcode sequence decoded was correctly identified and was not an error. In some cases, the quality score is a Phred-scaled quality value estimating the probability of incorrect call. In some embodiments, a probabilistic approach to decoding is used where each observed string of intensity in a neighborhood (e.g., a defined distance around a detected object) is matched to its most likely codeword with some probability and the probability is used to calculate a raw Q-score. In some cases, the raw Q-score is then refined by the negative controls for an adjusted Q-score. In some embodiments, a system is used to determine Phred scaled quality scores. The system may comprise a script, file, program, application, set of instructions, or computer-executable code that is configured to enable a computing device to calculate any one of the metrics described herein, e.g., statistics such as mean and median values, quality scores, etc. In some embodiments, the quality score is at least 20.

In some embodiments, the one or more pre-defined criteria can comprise at least or about 20%, at least or about 25%, at least or about 30%, at least or about 35%, at least or about 40%, at least or about 45%, at least or about 50%, at least or about 55%, at least or about 60%, at least or about 65%, or at least or about 70% of detected RCPs are correctly decoded (e.g., to a gene in the set of reference genes). In some embodiments, the one or more pre-defined criteria can comprise at least or about 20%, at least or about 25%, at least or about 30%, at least or about 35%, at least or about 40%, at least or about 45%, at least or about 50%, at least or about 55%, at least or about 60%, at least or about 65%, or at least or about 70% of detected RCPs are decoded (e.g., to a gene in the set of reference genes) with a quality score of at least 20. In some embodiments, the one or more pre-defined criteria can comprise at least 40% of detected RCPs are decoded with a quality score of at least 20. In some embodiments, the one or more pre-defined criteria can comprise at least 29% of detected RCPs are decoded with a quality score of at least 20. In some embodiments, the one or more pre-defined criteria can comprise that the fraction of objects decoded to gene with Q-score >=20 is at least 40%. In some embodiments, the one or more pre-defined criteria can comprise that the fraction of objects decoded to gene with Q-score >=20 is at least 29%. In some embodiments, the one or more pre-defined criteria can comprise at least 10 transcripts (e.g., RCPs are decoded to a gene in the set of reference genes) are decoded per 100 μm² region. In some embodiments, the one or more pre-defined criteria can comprise at least 15 transcripts are decoded per 100 μm² region.

In some embodiments, the one or more pre-defined criteria can comprise that the number of detected RCPs per μm² with a quality score of at least 20 is greater than 0, at least or about 0.0001, at least or about 0.00015, at least or about 0.0002, at least or about 0.00025, at least or about 0.0003, at least or about 0.00035, at least or about 0.0004, at least or about 0.00045, at least or about 0.0005, at least or about 0.00055, at least or about 0.0006, at least or about 0.00065, at least or about 0.0007, at least or about 0.00075, at least or about 0.0008, at least or about 0.00085, at least or about 0.0009, at least or about 0.00095, or at least or about 0.001. In some embodiments, the one or more pre-defined criteria can comprise that the detected gene density with a quality score of at least 20 is greater than 0, at least 0.0001, at least 0.0002, or at least 0.0003, at least 0.0004, at least 0.0005, at least 0.0006, at least 0.0007, at least 0.0008, at least 0.0009, or at least 0.001.

In some embodiments, the one or more pre-defined criteria can comprise that the thickness of detected RCPs with a quality score of at least 20 is less than or about 1.2 μm, less than or about 1.1 μm, less than or about 1.0 μm, less than or about 0.9 μm, less than or about 0.8 μm, less than or about 0.7 μm, less than or about 0.6 μm, less than or about 0.5 μm, or less than or about 0.4 μm.

In some embodiments, the one or more pre-defined criteria can comprise that the maximum decoding false positive rate is less than or about 25%, less than or about 20%, less than or about 15%, less than or about 10%, less than or about 5%, or less than about 2%. In some embodiments, the one or more pre-defined criteria can comprise that the maximum decoding false positive rate is less than 15%.

In some embodiments, the one or more pre-defined criteria can comprise that the maximum decoding false negative rate is less than or about 95%, less than or about 90%, less than or about 85%, less than or about 80%, less than or about 75%, less than or about 70%, less than or about 65%, less than or about 60%, less than or about 55%, less than or about 50%, less than or about 45%, or less than or about 40%. In some embodiments, the one or more pre-defined criteria can comprise that the maximum decoding false negative rate is less than 90%.

In some embodiments, the one or more pre-defined criteria can comprise that the minimal number of detected RCPs that are decoded with a quality score of at least 20 per field of view (FOV) is greater than 0, at least or about 1, at least or about 100, at least or about 1,000, at least or about 2,000, or at least or about 5,000. In some embodiments, the one or more pre-defined criteria can comprise that the minimal number of detected RCPs that are decoded (e.g., to a gene in a set of references genes) with a quality score of at least 20 per field of view (FOV) is between about 1 and about 100, between about 100 and bout 1,000, between about 1,000 and about 2,000, or between about 2,000 and about 5,000.

In some embodiments, the one or more pre-defined criteria can comprise that the quartile coefficient of dispersion of decoded different identifier sequences (e.g., corresponding to different genes in a set of references genes) with a quality score of at least 20 per field of view (FOV) is less than or about 75%, less than or about 70%, less than or about 65%, less than or about 60%, less than or about 55%, less than or about 50%, less than or about 45%, less than or about 40%, less than or about 35%, less than or about 30%, less than or about 25%, less than or about 20%, or less than or about 15%. In some embodiments, the one or more pre-defined criteria can comprise that the quartile coefficient of dispersion of decoded genes with a quality score of at least 20 per field of view (FOV) is less than 60%.

In some embodiments, the one or more pre-defined criteria can comprise that the number of particles detected is at least or about 200, at least or about 300, at least or about 400, at least or about 500, at least or about 600, at least or about 700, at least or about 800, at least or about 900, at least or about 1,000, at least or about 1,100, at least or about 1,200, at least or about 1,300, at least or about 1,400, at least or about 1,500, at least or about 1,600, at least or about 1,700, or at least or about 1,800. In some embodiments, the one or more pre-defined criteria can comprise that the number of particles detected (e.g., mimicking nuclei count) is at least 500.

In some embodiments, the one or more pre-defined criteria can comprise that the percent of transcripts (e.g., RCPs are decoded to a gene in the set of reference genes) that are detected within cells is at least or about 40%, at least or about 45%, at least or about 50%, at least or about 55%, at least or about 60%, at least or about 65%, at least or about 70%, at least or about 75%, at least or about 80%, at least or about 85%, at least or about 90%, or at least or about 95%. In some embodiments, the one or more pre-defined criteria can comprise that the percent of transcripts that are detected within cells is at least or about 80%. In some embodiments, the one or more pre-defined criteria can comprise that the percent of transcripts that are detected within cells is at least or about 95%. In some embodiments, association of a transcript with a cell is based on the particles detected (e.g., mimicking cells).

In some embodiments, the one or more pre-defined criteria can comprise any one or more of: at least or about 30% of detected RCPs are decoded with a quality score of at least 20; the number of detected RCPs per μm² with a quality score of at least 20 is greater than 0; the thickness of detected RCPs with a quality score of at least 20 is less than or about 1.2 μm; the maximum decoding false positive rate is less than or about 25%; the maximum decoding false negative rate is less than or about 85%; the minimal number of detected RCPs that are decoded with a quality score of at least 20 per field of view (FOV) is greater than 0; the quartile coefficient of dispersion of decoded different identifier sequences with a quality score of at least 20 per field of view (FOV) is less than or about 70%; and the number of particles detected is at least or about 300.

In some embodiments, the one or more pre-defined criteria can comprise any one or more of: at least or about 35% of detected RCPs are decoded; the number of detected RCPs per μm² with a quality score of at least 20 is greater than or about 0.0001; the thickness of detected RCPs with a quality score of at least 20 is less than or about 1 μm; the maximum decoding false positive rate is less than or about 20%; the maximum decoding false negative rate is less than or about 80%, the minimal number of detected RCPs that are decoded with a quality score of at least 20 per field of view (FOV) is greater than 0; the quartile coefficient of dispersion of decoded different identifier sequences with a quality score of at least 20 per field of view (FOV) is less than or about 65%; and the number of particles detected is at least or about 400.

In some embodiments, the one or more pre-defined criteria can comprise any one or more of: at least or about 40% of detected RCPs are decoded; the number of detected RCPs per μm² with a quality score of at least 20 is greater than or about 0.0003; the thickness of detected RCPs with a quality score of at least 20 is less than or about 0.8 μm; the maximum decoding false positive rate is less than or about 15%; the maximum decoding false negative rate is less than or about 90%, the minimal number of detected RCPs that are decoded with a quality score of at least 20 per field of view (FOV) is 1 or greater; the quartile coefficient of dispersion of decoded different identifier sequences with a quality score of at least 20 per field of view (FOV) is less than or about 60%; and the number of particles detected is at least or about 500.

In some embodiments, the one or more pre-defined criteria comprises the expected median number of transcripts detected per cell, number of cells detected, and number of transcripts decoded per area (e.g., per 100 μm²). In some embodiments, the one or more pre-defined criteria are to characterize an assay as pass (e.g., satisfactory performance) compared to expected results (e.g., minimum levels for passing). In some cases, if one or more pre-defined criteria are not satisfactory, a warning is provided to the user.

In some embodiments, when at least or about 90% of the different identifier sequences in the RCPs are decoded using the instrument, the instrument can pass the qualification test as suitable for decoding analytes in situ in the biological sample. In some embodiments, the method can comprise using the qualified instrument to decode analytes in situ in the biological sample.

In some aspects, the method comprises detecting particles (e.g., beads) labeled with a blue fluorescent dye. In some aspects, the qualifying of the instrument and workflow performed on the instrument comprises processing and analyzing the detected particles to mimic nuclei count and segmentation of cells. For example, an instrument workflow can be considered satisfactory (e.g., pass) if a plurality of metrics in all regions of interest are met for all targets (e.g., identifier sequence of the synthetic constructs to be determined). In some cases, an instrument workflow performed does not pass the qualifying test if any one of the regions of interest shows that no objects were detected and decoded to identifier sequences with a Q-score of at least 20.

### V. In Situ Assays

In some aspects, an instrument (e.g., subjected to a QC method disclosed herein) can be used to perform any suitable in situ assay that involves the use of various probes for analyte detection in a biological sample. In some embodiments, the RCPs of the QC slides described in Section II are used to represent RCPs in a biological sample that are associated with analytes (e.g., transcripts). In some embodiments, the probes directly or indirectly bind to analytes at locations in the biological sample, and signals associated with the probes can be detected at locations in the biological sample to indicate the locations of the analytes. In some embodiments, a plurality of probes (e.g., detectably labeled probes, and optionally intermediate probes that hybridize to the detectably labeled probes and directly or indirectly bind to analytes or products thereof) can be used for sequential hybridization and detection in order to generate a signal code sequence (e.g., a spatiotemporal signal signature) for analytes at each of multiple locations in the biological sample, and the signal code sequence can be compared to those in a codebook to decode an identifier sequence (e.g., a barcode sequence or an analyte sequence) corresponding to an analyte, thereby identifying analytes at multiple locations in the biological sample. The signal code sequence can comprise signal codes each corresponding to a signal (e.g., signals of different colors correspond to different signal codes) or absence thereof (e.g., dark) at a particular location in the biological sample.

In some aspects, in situ assay can use microscopy as a readout, e.g., nucleic acid sequencing, nucleic acid probe hybridization, or other detection or determination methods involving an optical readout. In some aspects, detection or determination of a sequence of one, two, three, four, five, or more nucleotides of a target nucleic acid is performed in situ in a cell in an intact tissue. In some embodiments, the assay comprises detecting the presence or absence of an amplification product (e.g., an RCP comprising a particular identifier sequence such as a barcode sequence corresponding to a gene). In some embodiments, the present disclosure provides methods for high-throughput profiling of a large number of targets in situ, such as transcripts and/or DNA loci, e.g., for detecting and/or quantifying nucleic acids and/or non-nucleic acid molecules (e.g., proteins) in cells, tissues, organs or organisms. In some embodiments, the hybridization of probes with the sample and/or detection steps during the in situ assay is performed on an instrument that has been subjected to a QC method disclosed herein, e.g., as disclosed in Section III.

A biological sample may comprise one or a plurality of analytes of interest. Methods for performing multiplexed assays to analyze two or more different analytes in a single biological sample are provided. In some aspects, provided herein is a method comprising analyzing biological targets based on in situ hybridization of probes comprising nucleic acid sequences. In some embodiments, the method comprises sequential hybridization of detectably-labelled oligonucleotides to barcoded nucleic acid molecules in a sample. The barcoded nucleic acid molecules can be a primary probe that binds to a biological target; a nucleic acid product (e.g., RCP) of a primary probe; an intermediate probe that directly or indirectly binds to a primary probe or a product thereof; or a nucleic acid product (e.g., RCP) of an intermediate probe.

In some aspects, an in situ hybridization based assay is used to localize and analyze nucleic acid sequences (e.g., a DNA or RNA molecule comprising one or more specific sequences of interest) within a biological sample, e.g., a portion or section of tissue or a single cell. In some embodiments, the in situ assay is used to analyze the presence, absence, an amount or level of mRNA transcripts (e.g., a transcriptome or a subset thereof, or mRNA molecules of interest) in a biological sample, while preserving spatial context. In some embodiments, the present disclosure provides compositions and methods for in situ hybridization using directly or indirectly labeled molecules, e.g., complementary DNA or RNA or modified nucleic acids, as probes that bind or hybridize to a target nucleic acids within a biological sample of interest.

Nucleic acid probes, in some examples, are labelled with radioisotopes, epitopes, hapten, biotin, or fluorophores, to enable detection of the location of specific nucleic acid sequences on chromosomes or in tissues. In some embodiments, probes are locus specific (e.g., gene specific) and bind or couple to specific regions of a chromosome. In alternative embodiments, probes are alphoid or centromeric repeat probes that bind or couple to repetitive sequences within each chromosome. Probes may also be whole chromosome probes (e.g., multiple smaller probes) that bind or couple to sequences along an entire chromosome.

In some embodiments, an in situ assay may comprise a method comprising DNA in situ hybridization to measure and localize DNA, such as cDNA or genomic DNA. In some embodiments, provided herein is a method RNA in situ hybridization to measure and localize RNAs (e.g., mRNAs, lncRNAs, and miRNAs) within a biological sample (e.g., a fixed tissue sample). In some embodiments, RNA in situ hybridization involves single-molecule RNA fluorescence in situ hybridization (FISH). In some embodiments, fluorescently labelled nucleic acid probes are hybridized to pre-determined RNA targets, to visualize gene expression in a biological sample. In some embodiments, a FISH method comprises using a single nucleic acid probe specific to each target, e.g., single-molecule FISH (smFISH). The use of smFISH may produce a fluorescence signal that allows for quantitative measurement of RNA transcripts. In some embodiments, smFISH comprises a set of nucleic acid probes, about 50 base pairs in length, wherein each probe is coupled to a set fluorophores. For example, the set of nucleic acid probes may comprise five probes, wherein each probe coupled to five fluorophores. In some embodiments, said nucleic acid probes are instead each coupled to one fluorophore. For example, a smFISH protocol may use a set of about 40 nucleic acid probes, about 20 base pairs in length, each coupled to a single fluorophore. In some embodiments, the length of the nucleic acid probes varies, comprising 10 to 100 base pairs, such as 30 to 60 base pairs. Alternatively, a plurality of nucleic acid probes targeting different regions of the same RNA transcript may be used. The type of nucleic acid probes, the number of nucleic acid probes, the number of fluorophores coupled to said probes, and the length of said probes, may be varied to fit the specifications of the individual assay.

In further embodiments, smFISH is applied to a multiplexed workflow where consecutive/sequential hybridizations are used (e.g., as in seqFISH or seqFISH+) to impart a temporal barcode on target transcripts. Sequential rounds of fluorescence in situ hybridization may be accompanied by imaging and probe stripping, detecting individual transcripts (e.g., RNA transcripts) within a biological sample of interest (e.g., a tissue sample, a single cell, or extracted RNA). In some embodiments, each round of hybridization comprises a pre-defined set of probes (e.g., between about 10 and about 50 probes such as 24 to 32 probes) that target unique RNA transcripts. In some examples, the pre-defined set of probes is multicolored. Optionally, multiple nucleic acid probes are attached onto the sample, wherein each probe comprises an initiation sequence for amplification, allowing for decreased autofluorescence (e.g., as in single-molecule hybridization chain reaction (smHCR)). In some embodiments, a multiplexed smFISH method described herein may multiplex from 10s to over 10,000 mRNAs, optionally accompanied by imaging, to efficiently and accurately profile the entire transcriptome. In situ hybridization methods may further comprise using two probes to bind target transcripts (e.g., RNA transcripts), that serve as binding targets for amplification primers. In some embodiments, this process results in signal amplification (e.g., as in RNAscope).

In some embodiments, an in situ assay may comprise a multiplexed FISH protocol that is error-robust (e.g., MERFISH). In some embodiments, said protocol comprises primary probes comprising a binding region (e.g., a region that binds to a target such as RNA transcripts) coupled to one or more flanking regions. In some embodiments, each primary probe comprises two flanking regions. The primary probes may hybridize to a transcript (e.g., RNA transcript) within a biological sample (e.g., tissue sample or a single cell), such that florescent readout nucleic acid probes may subsequently serially hybridize to the flanking region(s) of the primary probes. In some embodiments, each round of hybridization comprises successive imaging and probe stripping to quench signals from readout nucleic acid probes from previous rounds. RNAs may be imaged by FISH, and errors accumulated during multiple imaging rounds (e.g., imperfect hybridizations) are detected and/or corrected. In some embodiments, expansion microscopy is employed to increase the number of detected RNA targets without signal overlap. Barcoding may be performed, comprising sequential hybridizations using readout probes coupled to pre-determined colors to generate unique barcodes (e.g., generating pseudocolors from consecutive hybridizations).

In some embodiments, one or more barcodes of a probe are targeted by detectably labeled detection oligonucleotides, such as fluorescently labeled oligonucleotides. In some embodiments, one or more decoding schemes are used to decode the signals, such as fluorescence, for sequence determination. In some embodiments, barcodes (e.g., primary and/or secondary barcode sequences) are analyzed using any suitable methods or techniques, comprising those described herein, such as RNA sequential probing of targets (RNA SPOTs), sequential fluorescent in situ hybridization (seqFISH or seqFISH+), single-molecule fluorescent in situ hybridization (smFISH), or multiplexed error-robust fluorescence in situ hybridization (MERFISH). In some embodiments, the methods provided herein comprise analyzing the barcodes by sequential hybridization and detection with a plurality of labelled probes (e.g., detection oligonucleotides). Examples of decoding schemes are described in Eng et al., “Transcriptome-scale Super-Resolved Imaging in Tissues by RNA SeqFISH+,” Nature 568(7751):235-239 (2019); Chen et al., “Spatially resolved, highly multiplexed RNA profiling in single cells,” Science; 348(6233): aaa6090 (2015); U.S. Pat. No. 10,457,980 B2; US 2016/0369329 A1; WO 2018/026873 A1; US 2021/0017587 A1; and US 2017/0220733 A1, all of which are incorporated by reference in their entirety. In some embodiments, these assays enable signal amplification, combinatorial decoding, and error correction schemes at the same time.

In some embodiments, an in situ assay may comprise linking sequencing information and spatial information of targets within endogenous environments. For example, analysis of nucleic acid sequences may be performed directly on DNA or RNA within a biological sample of interest. In some embodiments, the present disclosure allows for the simultaneous identification and quantification of a plurality of targets, such as 100s, 1000s, or more of transcripts (e.g., mRNA transcripts), in addition to spatial resolution of said transcripts. In some aspects, the spatial resolution of transcripts is subcellular. Optionally, the spatial resolution may be increased using signal amplification strategies described herein.

In some embodiments, circularizable probes or probe sets (e.g., padlock probes)comprise oligonucleotides with ends that are complementary to a target sequence (e.g., target RNA or DNA). Upon hybridization of circularizable probes or probe sets to the target sequence, enzymes may be used to ligate the ends of the circularizable probes or probe sets, and catalyze the formation of circularized probes.

In some aspects, an in situ assay may comprise using nucleic acid probes and/or probe sets and immobilization oligonucleotides that are introduced into a cell or used to otherwise contact a biological sample such as a tissue sample. The probes may comprise any one of a variety of entities that can hybridize to a nucleic acid, typically by Watson-Crick base pairing, such as DNA, RNA, LNA, PNA, etc., depending on the application. The nucleic acid probe(s) and immobilization oligonucleotides typically contains a hybridization region that is able to bind to at least a portion of a target nucleic acid, in some embodiments specifically. The nucleic acid probe may be able to bind to a specific target nucleic acid (e.g., an mRNA, or other nucleic acids as discussed herein). In some embodiments, the nucleic acid probes are detected using a detectable label, and/or by using detectably labeled nucleic acid probes able to bind to the nucleic acid probes or amplification products thereof, directly or via an intermediate probe. In some embodiments, the nucleic acid probes are compatible with one or more biological and/or chemical reactions. For instance, a primary nucleic acid probe disclosed herein can serve as a template or primer for a polymerase (e.g., for rolling circle amplification), a template or substrate for a ligase, a substrate for a click chemistry reaction, and/or a substrate for a nuclease (e.g., endonuclease for cleavage).

In some cases, a probe or probe set is a barcoded probe or probe set. Examples of barcoded probes or probe sets may comprise a circularizable probe or probe set (e.g., based on a padlock probe, a gapped padlock probe, a SNAIL (Splint Nucleotide Assisted Intramolecular Ligation) probe set), a PLISH (Proximity Ligation in situ Hybridization) probe set, a RollFISH probe set, or a PLAYR (Proximity Ligation Assay for RNA) probe set). In some embodiments, a barcoded probe or probe set is not circular or circularizable. Examples of barcoded probes or probe sets include, but are not limited to, L-shaped probes (e.g., a probe comprising a target-hybridizing sequence and a 5′ or 3′ overhang upon hybridization to its target sequence), or U-shaped probes (e.g., a probe comprising a target-hybridizing sequence and a 5′ overhang and a 3′ overhang upon hybridization to its target sequence). The specific probe or probe set design can vary.

In some embodiments, a probe or probe set comprises a probe comprising a 3′ or 5′ overhang upon hybridization to the target nucleic acid (e.g., an L-shaped probe). In some embodiments, the overhang comprises one or more barcode sequences corresponding to the target nucleic acid (e.g., the target RNA transcript). In some embodiments, a plurality of probes are designed to hybridize to the target nucleic acid (e.g., at least 20, 30, or 40 probes can hybridize to the target nucleic acid). In some embodiments, the probe or probe set is a probe comprising a 3′ overhang and a 5′ overhang upon hybridization to the target nucleic acid (a U-shaped probe). In some embodiments, the 3′ overhang and the 5′ overhang each independently comprises one or more detectable labels and/or barcode sequences. In some embodiments, the 3′ and/or 5′ overhang comprises one or more detectable labels and/or barcode sequences.

In some aspects, an in situ assay may comprise analyzing, e.g., detecting or determining, one or more sequences present in the probes or probe sets or products thereof (e.g., rolling circle amplification products thereof). In some embodiments, the detecting is performed at one or more locations in a biological sample. In some embodiments, the locations are the locations of RNA transcripts in the biological sample. In some embodiments, the locations are the locations at which the probes or probe sets hybridize to the RNA transcripts in the biological sample, and are optionally ligated and amplified by rolling circle amplification.

In some embodiments, the detecting comprises a plurality of repeated cycles of hybridization and removal of probes (e.g., detectably labeled probes, or intermediate probes that bind to detectably labeled probes) to the primary probe or probe set hybridized to the target nucleic acid, or to a rolling circle amplification product generated from the probe or probe set hybridized to the target nucleic acid.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting can comprise binding an intermediate probe directly or indirectly to the primary probe or probe set, binding a detectably labeled probe directly or indirectly to a detection region of the intermediate probe, and detecting a signal associated with the detectably labeled probe. In some embodiments, the method comprises detecting a rolling circle amplification product (RCP) generated using a circular or circularized primary probe or probe set as a template. In some embodiments, the method comprises detecting a rolling circle amplification product (RCP) generated using a circular or circularized probe or probe that binds to a primary probe or probe set as a template. In some embodiments, detecting the RCP comprises binding an intermediate probe directly or indirectly to the RCP, binding a detectably labeled probe directly or indirectly to a detection region of the intermediate probe, and detecting a signal associated with the detectably labeled probe. In some embodiments, the method can comprise performing one or more wash steps to remove unbound and/or nonspecifically bound intermediate probe molecules from the primary probes or the products of the primary probes.

In some embodiments, the detecting can comprise: detecting signals associated with detectably labeled probes that are hybridized to barcode regions or complements thereof in the primary probe or probe set or a product thereof (e.g., an RCP); and/or detecting signals associated with detectably labeled probes that are hybridized to intermediate probes which are in turn hybridized to the barcode regions or complements thereof. In some embodiments, the detectably labeled probes are fluorescently labeled.

In some embodiments, the methods comprise detecting the sequence in all or a portion of a primary probe or probe set or an RCP, or detecting a sequence of the primary probe or probe set or RCP, such as one or more barcode sequences present in the primary probe or probe set or RCP. In some embodiments, the sequence of the RCP, or barcode thereof, is indicative of a sequence of the target nucleic acid to which the RCP is hybridized. In some embodiments, the analysis and/or sequence determination comprises detecting a sequence in all or a portion of the nucleic acid concatemer and/or in situ hybridization to the RCP. In some embodiments, the detection step involves sequencing by hybridization, sequencing by ligation, sequencing by synthesis, sequencing by binding, and/or fluorescent in situ sequencing (FISSEQ), and/or hybridization-based in situ sequencing. In some embodiments, the detection step is by sequential fluorescent in situ hybridization (e.g., for combinatorial decoding of the barcode sequence or complement thereof). In some embodiments, the analysis and/or sequence determination comprises detecting a polymer generated by a hybridization chain reaction (HCR) reaction. In some embodiments, the detection or determination comprises hybridizing to the first overhang a detection oligonucleotide labeled with a fluorophore, an isotope, a mass tag, or a combination thereof. In some embodiments, the detection or determination comprises imaging the probe hybridized to the target nucleic acid (e.g., imaging one or more detectably labeled probes hybridized thereto). In some embodiments, the target nucleic acid is an mRNA in a tissue sample, and the detection or determination is performed when the target nucleic acid and/or the amplification product is in situ in the tissue sample. In some embodiments, the target nucleic acid is an amplification product (e.g., a rolling circle amplification product).

In some aspects, the provided methods comprise imaging a detectably labeled probe bound directly or indirectly to the primary probe or probe set or product thereof and detecting the detectable label. In some embodiments, the detectably labeled probe comprises a detectable label that can be measured and quantitated. The label or detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (Max Vision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). Non-limiting examples of techniques and methods applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Examples of fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles are used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence is/are indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Examples of haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some aspects, the detecting involves using detection methods such as flow cytometry; sequencing; probe binding and electrochemical detection; pH alteration; catalysis induced by enzymes bound to DNA tags; quantum entanglement; Raman spectroscopy; terahertz wave technology; and/or scanning electron microscopy. In some aspects, the flow cytometry is mass cytometry or fluorescence-activated flow cytometry. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any one of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECS™), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PS™, photon scanning tunneling microscopy (PS™), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXS™), and intact tissue expansion microscopy (exM).

In some embodiments, the assay comprises in situ sequencing. In situ sequencing typically involves incorporation of a labeled nucleotide (e.g., fluorescently labeled mononucleotides or dinucleotides) in a sequential, template-dependent manner or hybridization of a labeled primer (e.g., a labeled random hexamer) to a nucleic acid template such that the identities (e.g., nucleotide sequence) of the incorporated nucleotides or labeled primer extension products can be determined, and consequently, the nucleotide sequence of the corresponding template nucleic acid. Aspects of in situ sequencing are described, for example, in Mitra et al., (2003) Anal. Biochem. 320, 55-65, and Lee et al., (2014) Science, 343(6177), 1360-1363. In addition, examples of methods and systems for performing in situ sequencing are described in US 2016/0024555, US 2019/0194709, and in U.S. Pat. Nos. 10,138,509, 10,494,662 and 10,179,932, all of which are herein incorporated by reference in their entireties. Examples of techniques for in situ sequencing or in situ sequence detection comprise, but are not limited to, STARmap (described for example in Wang et al., (2018) Science, 361(6499) 5691), MERFISH (described for example in Moffitt, (2016) Methods in Enzymology, 572, 1-49), hybridization-based in situ sequencing (HybISS) (described for example in Gyllborg et al., Nucleic Acids Res (2020) 48(19):e112, and FISSEQ (described for example in US 2019/0032121, the content is herein incorporated by reference in its entirety).

In some embodiments, sequencing is performed by sequencing-by-synthesis (SBS). In some embodiments, a sequencing primer is complementary to sequences at or near the one or more barcode(s). In such embodiments, sequencing-by-synthesis can comprise reverse transcription and/or amplification in order to generate a template sequence from which a primer sequence can bind. Examples of SBS methods comprise those described for example, but not limited to, US 2007/0166705, US 2006/0188901, U.S. Pat. No. 7,057,026, US 2006/0240439, US 2006/0281109, US 2011/0059865, US 2005/0100900, U.S. Pat. No. 9,217,178, US 2009/0118128, US 2012/0270305, US 2013/0260372, and US 2013/0079232, all of which are herein incorporated by reference in their entireties.

In some embodiments, sequencing is performed by sequencing-by-binding (SBB). Various aspects of SBB are described in U.S. Pat. No. 10,655,176 B2, the content of which is herein incorporated by reference in its entirety. In some embodiments, SBB comprises performing repetitive cycles of detecting a stabilized complex that forms at each position along the template nucleic acid to be sequenced (e.g. a ternary complex that includes the primed template nucleic acid, a polymerase, and a cognate nucleotide for the position), under conditions that prevent covalent incorporation of the cognate nucleotide into the primer, and then extending the primer to allow detection of the next position along the template nucleic acid. In the sequencing-by-binding approach, detection of the nucleotide at each position of the template occurs prior to extension of the primer to the next position. Generally, the methodology is used to distinguish the four different nucleotide types that can be present at positions along a nucleic acid template by uniquely labelling each type of ternary complex (e.g., different types of ternary complexes differing in the type of nucleotide it contains) or by separately delivering the reagents needed to form each type of ternary complex. In some instances, the labeling may comprise fluorescence labeling of, e.g., the cognate nucleotide or the polymerase that participate in the ternary complex.

In some embodiments, sequencing is performed by sequencing-by-avidity (SBA). Some aspects of SBA approaches are described in U.S. Pat. No. 10,768,173 B2, the content of which is herein incorporated by reference in its entirety. In some embodiments, SBA comprises detecting a multivalent binding complex formed between a fluorescently-labeled polymer-nucleotide conjugate, and a one or more primed target nucleic acid sequences (e.g., barcode sequences). Fluorescence imaging is used to detect the bound complex and thereby determine the identity of the N+1 nucleotide in the target nucleic acid sequence (where the primer extension strand is N nucleotides in length). Following the imaging step, the multivalent binding complex is disrupted and washed away, the correct blocked nucleotide is incorporated into the primer extension strand, and the sequencing cycle is repeated.

In some embodiments, detection of the barcode sequences is performed by sequential hybridization of probes to the barcode sequences or complements thereof and detecting complexes formed by the probes and barcode sequences or complements thereof. In some cases, each barcode sequence or complement thereof is assigned a sequence of signal codes that identifies the barcode sequence or complement thereof (e.g., a temporal signal signature or code that identifies the analyte), and detecting the barcode sequences or complements thereof can comprise decoding the barcode sequences of complements thereof by detecting the corresponding sequences of signal codes detected from sequential hybridization, detection, and removal of sequential pools of intermediate probes and the universal pool of detectably labeled probes. In some cases, the sequences of signal codes are fluorophore sequences assigned to the corresponding barcode sequences or complements thereof. In some embodiments, the detectably labeled probes are fluorescently labeled. In some embodiments, the barcode sequence or complement thereof is decoded by sequential probe hybridization as described in US 2021/0340618, the content of which is herein incorporated by reference in its entirety.

In some embodiments, the detecting step comprises contacting the biological sample with one or more detectably labeled probes that directly or indirectly hybridize to the barcode sequences or complements thereof (e.g., in amplification products generated using the probes or probe sets), and dehybridizing the one or more detectably labeled probes. In some embodiments, the contacting and dehybridizing steps are repeated with the one or more detectably labeled probes and/or one or more other detectably labeled probes that directly or indirectly hybridize to the barcode sequences or complements thereof. In some aspects, the method comprises sequential hybridization of detectably labeled probes to create a spatiotemporal signal signature or code that identifies the analyte.

In some embodiments, the detecting step comprises contacting the biological sample with one or more first detectably labeled probes that directly hybridize to the plurality of probes or probe sets. In some instances, the detecting step can comprise contacting the biological sample with one or more first detectably labeled probes that indirectly hybridize to the plurality of probes or probe sets. In some embodiments, the detecting step comprises contacting the biological sample with one or more second detectably labeled probes that directly or indirectly hybridize to the plurality of probes or probe sets.

In some embodiments, the detecting step comprises contacting the biological sample with one or more intermediate probes that directly or indirectly hybridize to the barcode sequences or complements thereof (e.g., of the plurality of probes or probe sets or rolling circle amplification product generated using the plurality of probes or probe sets), wherein the one or more intermediate probes are detectable using one or more detectably labeled probes. In some embodiments, the detecting step further comprises dehybridizing the one or more intermediate probes and/or the one or more detectably labeled probes from the barcode sequences or complements thereof (e.g., of the plurality of probes or probe sets or rolling circle amplification product generated using the plurality of probes or probe sets). In some embodiments, the contacting and dehybridizing steps are repeated with the one or more intermediate probes, the one or more detectably labeled probes, one or more other intermediate probes, and/or one or more other detectably labeled probes.

In some embodiments, sequencing is performed using sequencing by ligation. Such techniques utilize DNA ligase to incorporate oligonucleotides and identify the incorporation of such oligonucleotides. The oligonucleotides typically have different labels that are correlated with the identity of a particular nucleotide in a sequence to which the oligonucleotides hybridize. Aspects and features involved in sequencing by ligation are described, for example, in Shendure et al. Science (2005), 309: 1728-1732, and in U.S. Pat. Nos. 5,599,675; 5,750,341; 6,969,488; 6,172,218; and 6,306,597, all of which are herein incorporated by reference in their entireties.

In some embodiments, nucleic acid hybridization is used for sequencing. These methods utilize labeled nucleic acid decoder probes that are complementary to at least a portion of a barcode sequence. Multiplex decoding can be performed with pools of many different probes with distinguishable labels. Non-limiting examples of nucleic acid hybridization sequencing are described for example in U.S. Pat. No. 8,460,865, and in Gunderson et al., Genome Research 14:870-877 (2004), all of which are herein incorporated by reference in their entireties.

In some embodiments, real-time monitoring of DNA polymerase activity is used during sequencing. For example, nucleotide incorporations can be detected through fluorescence resonance energy transfer (FRET), as described for example in Levene et al., Science (2003), 299, 682-686, Lundquist et al., Opt. Lett. (2008), 33, 1026-1028, and Korlach et al., Proc. Natl. Acad. Sci. USA (2008), 105, 1176-1181.

The analysis and/or sequence determination can be carried out at room temperature for best preservation of tissue morphology with low background noise and error reduction. In some embodiments, the analysis and/or sequence determination comprises eliminating error accumulation as sequencing proceeds.

In some embodiments, the analysis and/or sequence determination involves washing to remove unbound polynucleotides, thereafter revealing a fluorescent product for imaging.

A sample disclosed herein for in situ analysis can be derived from any biological sample. The sample may not be limited to any specific source, but may be peripheral blood mononuclear cells, tumors, tissue, bone marrow, biopsies, serum, blood, plasma, saliva, lymph fluid, pleura fluid, cerebrospinal and synovial fluid. The sample may be extracted from a subject. Samples extracted from individuals may be subjected to the methods described herein to identify and evaluate immune responses during cancer and disease or subsequent to immunotherapy.

Methods and compositions disclosed herein may be used for analyzing a biological sample, which may be obtained from a subject using any one of a variety of techniques including, but not limited to, biopsy, surgery, and laser capture microscopy (LCM), and generally includes cells and/or other biological material from the subject. A biological sample can also be obtained from a eukaryote, such as a tissue sample, a patient derived organoid (PDO) or patient derived xenograft (PDX). In some embodiments, the biological sample is obtained from a cell block or a cell pellet, A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components.

Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

In some embodiments, the biological sample corresponds to cells (e.g., derived from a cell culture, a tissue sample, or cells deposited on a surface). In a cell sample with a plurality of cells, individual cells can be naturally unaggregated. For example, the cells can be derived from a suspension of cells (e.g., a body fluid such as blood) and/or disassociated or disaggregated cells from a tissue or tissue section. The number of cells in the biological sample can vary. Some biological samples comprise large numbers of cells, e.g., blood samples, while other biological samples comprise smaller or only a small number of cells or may only be suspected of containing cells, e.g., plasma, serum, urine, saliva, synovial fluids, amniotic fluid, lachrymal fluid, lymphatic fluid, liquor, cerebrospinal fluid and the like.

In some embodiments, a cell-containing biological sample can comprise a body fluid or a cell-containing sample derived from the body fluid, e.g., whole blood, samples derived from blood such as plasma or serum, buffy coat, urine, sputum, lachrymal fluid, lymphatic fluid, sweat, liquor, cerebrospinal fluid, ascites, milk, stool, bronchial lavage, saliva, amniotic fluid, nasal secretions, vaginal secretions, semen/seminal fluid, wound secretions, cell culture and swab samples, or any cell-containing sample derived from the aforementioned samples. In some embodiments, a cell-containing biological sample can be a body fluid, a body secretion or body excretion, e.g., lymphatic fluid, blood, buffy coat, plasma or serum. In some embodiments, a cell-containing biological sample can be a circulating body fluid such as blood or lymphatic fluid, e.g., peripheral blood obtained from a mammal such as human.

The biological sample can include any number of macromolecules, for example, cellular macromolecules and organelles (e.g., mitochondria and nuclei). The biological sample can be obtained as a tissue sample, such as a tissue section, biopsy, a core biopsy, needle aspirate, or fine needle aspirate. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface. In some embodiments, the biological sample may comprises transcripts of antigen receptor molecules.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

A substrate herein can be any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate is coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section is prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) is prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample is prepared using formalin-fixation and paraffin-embedding (FFPE), which are established methods. In some embodiments, cell suspensions and other non-tissue samples are prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any one of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as a circular probe or a circularizable probe or probe set (e.g., a padlock probe). In some embodiments, one or more post-fixing step is performed after a hybridization complex comprising a probe and a target is formed in a sample. In some embodiments, one or more post-fixing step is performed prior to a ligation reaction disclosed herein, such as the ligation to circularize a circularizable probe or probe set (e.g., a padlock probe).

In some embodiments, one or more post-fixing step is performed after contacting a sample with a binding or labelling agent (e.g., an antibody or antigen binding fragment thereof) for a non-nucleic acid analyte such as a protein analyte. The labelling agent can comprise a nucleic acid molecule (e.g., reporter oligonucleotide) comprising a sequence corresponding to the labelling agent and therefore corresponds to (e.g., uniquely identifies) the analyte. In some embodiments, the labelling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any one of a variety of other embedding materials to provide structural substrate to the sample prior to sectioning and other handling steps. In some cases, the embedding material is removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, a sample is stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain is specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample are segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DIR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample is stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample is stained using Romanowsky stain, including Wright's stain, Jenner's stain, Can-Grunwald stain, Leishman stain, and Giemsa stain.

In some embodiments, biological samples are destained. Various methods of destaining or discoloring a biological sample can be used, and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) is isometrically expanded. Isometric expansion methods that can be used include hydration, a preparative step in expansion microscopy, as described in Chen et al., Science 347(6221):543-548, 2015.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto is/are anchored to a polymer matrix. For example, the polymer matrix can be a hydrogel. In some embodiments, one or more of the polynucleotide probe(s) and/or amplification product (e.g., amplicon) thereof is/are modified to contain functional groups that can be used as an anchoring site to attach the polynucleotide probes and/or amplification product to a polymer matrix. In some embodiments, a modified probe comprising oligo dT is used to bind to mRNA molecules of interest, followed by reversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel can include hydrogel subunits, such as, but not limited to, acrylamide, bis-acrylamide, polyacrylamide and derivatives thereof, poly(ethylene glycol) and derivatives thereof (e.g., PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry can be used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labelling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample is permeabilized to facilitate transfer of species (such as probes) into the sample. If a sample is not permeabilized sufficiently, the amount of species (such as probes) in the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample is incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample is permeabilized by non-chemical permeabilization methods. Non-chemical permeabilization methods can be used. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

(ix) Selective Enrichment of RNA or cDNA Species

In some embodiments, where RNA or cDNA is the analyte, one or more RNA or cDNA analyte species of interest is/are selectively enriched. For example, one or more species of RNA or cDNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs or cDNAs of interest can be used to amplify the one or more RNAs or cDNAs of interest, thereby selectively enriching these RNAs or cDNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labelling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include amplification of templated ligation products (e.g., by multiplex PCR).

In some embodiments, the analytes are further enriched for in situ readout by immobilization at a location in the biological sample. In a non-limiting example, the analytes may comprise one or more fragments that are specific to a location in the biological sample.

Alternatively, one or more species of RNA can be down-selected (e.g., removed) using any one of a variety of methods. For example, probes can be administered to a sample that selectively hybridize to ribosomal RNA (rRNA), thereby reducing the pool and concentration of rRNA in the sample. Additionally and alternatively, duplex-specific nuclease (DSN) treatment can remove rRNA (see, e.g., Archer, et al, Selective and flexible depletion of problematic sequences from RNA-seq libraries at the cDNA stage, BMC Genomics, 15 401, (2014), the entire contents of which are incorporated herein by reference). Furthermore, hydroxyapatite chromatography can remove abundant species (e.g., rRNA) (see, e.g., Vandernoot, V. A., cDNA normalization by hydroxyapatite chromatography to enrich transcriptome diversity in RNA-seq applications, Biotechniques, 53(6) 373-80, (2012), the entire contents of which are incorporated herein by reference).

### VI. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide” and “nucleic acid molecule”, used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide is a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such suitable buffers. Hybridization temperatures can be as low as 5° C., but are typically greater than 22° ° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids can be used. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Examples of stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any one of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42ºC, followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° ° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° ° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions are well known to those of skill in the art and are described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

In some embodiments, “primer” used herein is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

“Ligation” may refer, in some embodiments, to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, e.g., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif.; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

“Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXEMPLARY EMBODIMENTS

In some embodiments, the invention is a kit, comprising: a plurality of rolling circle amplification products (RCPs), a plurality of particles, and a solid support comprising functional groups for immobilizing the plurality of RCPs and/or the plurality of particles on the solid support.

In some embodiments, the plurality of RCPs are immobilized on the solid support, and/or the plurality of particles are immobilized on the solid support. In some embodiments, the RCPs are generated outside a cell or tissue sample, generated in solution, and/or generated in vitro. In some embodiments, the RCPs are not in a cell or tissue. In some embodiments, the RCPs are in solution or lyophilized. Optionally, in some embodiments, the kit comprises one or more vials and each vial comprises RCPs in solution or lyophilized RCPs. In some embodiments, each RCP comprises multiple copies of an identifier sequence. In some embodiments, each RCP is associated with an assigned signal code sequence from a codebook. In some embodiments, the ratio between the number of the particles and the number of RCPs is about 1:250 or lower, about 1:500 or lower, about 1:750 or lower, or about 1:1,000 or lower. In some embodiments, the plurality of RCPs comprise modified nucleic acid residues. Optionally, in some embodiments, amine-modified nucleic acid residues are incorporated during rolling circle amplification.

In some embodiments, the plurality of particles comprise beads coupled to detectable labels. In some embodiments, the plurality of particles comprise round beads and/or oval beads. In some embodiments, the beads are amine-modified. In some embodiments, the beads are latex beads. Optionally, in some embodiments, the beads are amine-modified polystyrene beads. In some embodiments, the diameters of the beads are between about 0.5 μm and about 3 μm. In some embodiments, the mean diameter of the beads is about 2 μm. In some embodiments, the size and/or shape of an individual bead is comparable to that of a nucleus in a cell or tissue sample. In some embodiments, the signal intensity of detectable labels on an individual bead is comparable to the signal intensity of a nucleus detected in a cell or tissue sample.

In some embodiments, the detectable labels have an excitation wavelength between about 300 nm and about 400 nm. Optionally, in some embodiments, the detectable labels have a maximum excitation wavelength between about 325 nm and about 375 nm. Optionally, in some embodiments, the detectable labels have a maximum excitation wavelength of about 350 or about 360 nm. In some embodiments, the detectable labels have an emission wavelength between about 400 nm and about 600 nm. Optionally, in some embodiments, the detectable labels have a maximum emission wavelength of about 460 nm. In some embodiments, the detectable labels are non-autofluorescent or substantially nonfluorescent under excitation wavelengths of about 490 nm, about 530 nm, about 590 nm, and/or about 650 nm. In some embodiments, the detectable labels comprise a fluorescent DNA stain. Optionally, in some embodiments, the detectable labels comprise DAPI.

In some embodiments, the solid support comprises a planar substrate. Optionally, in some embodiments, the solid support comprises a glass or plastic substrate. In some embodiments, the plurality of RCPs and/or the plurality of particles are immobilized in two, three, four, or more discrete regions on the solid support. In some embodiments, the plurality of RCPs and/or the plurality of particles comprise a functional group configured to react with the functional groups of the solid support. Optionally, in some embodiments, the functional group of the plurality of RCPs and/or the plurality of particles comprises an amine and the functional groups of the solid support comprise an N-hydroxysuccinimide (NHS) moiety.

In some embodiments, herein is provided a slide comprising a solid support which has disposed thereon: rolling circle amplification products (RCPs) that are not in a cell or tissue, and a plurality of particles. Optionally, in some embodiments, the RCPs and the particles are randomly disposed in one, two, three, four, or more discrete regions on the solid support.

In some embodiments, the kit presented herein comprises detectably labeled probes configured to hybridize to the identifier sequences in the RCPs. In some embodiments, the kit presented herein comprises intermediate probes configured to hybridize to the identifier sequences in the RCPs, and detectably labeled probes configured to hybridize to at least some of the intermediate probes. In some embodiments, each intermediate probe comprises a recognition sequence configured to hybridize to one of the identifier sequences, and a hybridization sequence configured to hybridize to one of the detectably labeled probes. In some embodiments, the hybridization sequence is in a 3′ overhang or 5′ overhang of the intermediate probe. In some embodiments, the detectably labeled probes are detectable under excitation wavelengths of about 490 nm, about 530 nm, about 590 nm, or about 650 nm.

In some embodiments, one or more of the identifier sequences is a sequence of a gene or complement thereof. In some embodiments, one or more of the identifier sequences is a barcode sequence corresponding to a sequence of a gene or complement thereof. In some embodiments, the number of different identifier sequences in the RCPs is at least 4, at least 6, at least 8, at least 10, at least 12, at least 14, at least 16, or more. In some embodiments, the number of different detectably labeled probes is 3, 4, 5, 6, 7, or 8. In some embodiments, the number of different detectably labeled probes is 4, and the number of different identifier sequences in the RCPs is 9 or more. In some embodiments, the identifier sequences in the RCPs comprise sequences of a set of reference genes or complements thereof, and/or barcode sequences corresponding to a set of reference genes or complements thereof. In some embodiments, the codebook comprises signal code sequences each corresponding to a reference gene of the set of reference genes.

In some embodiments, herein is provided a method for qualifying an instrument, comprising placing a solid support on the instrument. In some embodiments, the instrument comprises: reagents comprising fluorescently labeled probes, a fluidics module, an optics module, and a system controller. In some embodiments, the reagents further comprise intermediate probes configured to hybridize to the identifier sequences in the RCPs, and the fluorescently labeled probes are configured to hybridize to the intermediate probes. In some embodiments, the instrument comprises a sample module configured to receive (and, optionally, secure) the solid support and/or the biological sample. In some embodiments, the biological sample is a cell or tissue sample. Optionally, in some embodiments, the biological sample is a tissue section. In some embodiments, the biological sample is a matrix-embedded biological sample. In some embodiments, the biological sample is a cleared biological sample. In some embodiments, the instrument comprises an ancillary module configured to facilitate operation of the instrument. Optionally, in some embodiments, the ancillary module comprises a cooling system and/or a motion calibration system. In some embodiments, the system controller controls operation of the fluidics module, the optics module, the sample module, and/or the ancillary module. In some embodiments, the system controller comprises a processor, a computer, and/or a computing platform. In some embodiments, the processor, the computer, and/or the computing platform are integrated, or are separate components configured to communicate with one another via a network. In some embodiments, the system controller comprises or is configured to communicate with a cloud computing platform. In some embodiments, the system controller is communicatively coupled with a data storage, an input device, a display system, or a combination thereof.

In some embodiments, the solid support comprises rolling circle amplification products (RCPs) disposed thereon, each RCP comprising multiple copies of an identifier sequence having an assigned signal code sequence from a codebook. In some embodiments, the number of different identifier sequences in the RCPs is at least 9, the number of fluorescently labeled probes of different sequences is 4, and the number of the sequential cycles is 4. In some embodiments, the solid support comprises a plurality of particles disposed thereon and does not comprise a cell or tissue sample thereon. In some embodiments, the plurality of particles are coupled to a fluorescent moiety. Optionally, in some embodiments, the fluorescent moiety has a maximum excitation wavelength of about 350 nm. Optionally, in some embodiments, the fluorescent moiety comprises DAPI. In some embodiments, the RCPs and the plurality of particles are disposed in two, three, four, or more discrete regions on the solid support. In some embodiments, the sizes and/or shapes of the particles are comparable to those of cell nuclei in a cell or tissue sample. In some embodiments, the mean diameter of the particles is about 2 μm and the particles are round or oval. In some embodiments, the signal intensity of an individual particle on the solid support is comparable to the signal intensity of a nucleus detected in the biological sample. In some embodiments, the ratio between the number of the particles and the number of the RCPs is between about 1:500 and about 1:5,000. In some embodiments, the ratio between the number of the particles and the number of the RCPs is about 1:1,000.

In some embodiments, the method provided herein further comprises using the fluidics module to deliver, in sequential cycles, the fluorescently labeled probes to the solid support. In some embodiments, the method provided herein further comprises using the optics module to detect, in the sequential cycles, signals (or absence thereof) associated with the fluorescently labeled probes directly or indirectly bound to the identifier sequences in the RCPs, thereby generating signal code sequences for the RCPs. In some embodiments, the method provided herein further comprises using the system controller to compare the generated signal code sequences to those from the codebook, thereby decoding the identifier sequences in the RCPs.

In some embodiments, the method provided herein further comprises qualifying the instrument, wherein the instrument is suitable for detecting analytes in a biological sample when the decoding in the decoding step (i.e., in the step of using the system controller to compare the generated signal code sequences to those from the codebook, thereby decoding the identifier sequences in the RCPs) meets one or more pre-defined criteria.

In some embodiments, the one or more pre-defined criteria comprise that at least or about 80%, at least or about 90%, or at least or about 95% of different identifier sequences in the RCPs are decoded in the decoding step (i.e., in the step of using the system controller to compare the generated signal code sequences to those from the codebook, thereby decoding the identifier sequences in the RCPs). In some embodiments, at least 90% of different identifier sequences in the RCPs are decoded in the decoding step, and the instrument is used to detect analytes in the biological sample.

In some embodiments, the one or more pre-defined criteria comprise any one or more of: at least or about 35% of detected RCPs are decoded; the number of detected RCPs per μm² with a quality score of at least q20 is greater than or about 0.0001; the thickness of detected RCPs with a quality score of at least q20 is less than or about 1 μm, the maximum decoding false positive rate is less than or about 20%; the maximum decoding false negative rate is less than or about 80%; the quartile coefficient of dispersion of decoded different identifier sequences with a quality score of at least q20 per field of view (FOV) is less than or about 65%; and the number of particles detected is at least or about 400.

In some embodiments, the one or more pre-defined criteria comprise any one or more of: at least or about 40% of detected RCPs are decoded; the number of detected RCPs per μm? with a quality score of at least q20 is greater than or about 0.0003; the thickness of detected RCPs with a quality score of at least q20 is less than or about 0.8 μm; the maximum decoding false positive rate is less than or about 15%; the maximum decoding false negative rate is less than or about 90%; the minimal number of detected RCPs that are decoded with a quality score of at least q20 is 1 or greater; the quartile coefficient of dispersion of decoded different identifier sequences with a quality score of at least q20 per field of view (FOV) is less than or about 60%; and the number of particles detected is at least or about 500.

In some embodiments, herein is provided a method of qualifying an instrument, comprising placing a solid support on the instrument. In some embodiments, the instrument comprises: reagents comprising intermediate probes and fluorescently labeled probes, a fluidics module, an optics module, and a system controller. In some embodiments, the solid support comprises rolling circle amplification products (RCPs) immobilized thereon, each RCP comprising multiple copies of an identifier sequence having an assigned signal code sequence from a codebook. In some embodiments, the method provided herein further comprises: in a first cycle, using the fluidics module to deliver to the solid support a first plurality of intermediate probe/fluorescently labeled probe pairs. In some embodiments, the intermediate probe and the fluorescently labeled probe in each pair form a complex comprising the intermediate probe hybridized to an RCP of the plurality of RCPs and the fluorescently labeled probe hybridized to the intermediate probe. In some embodiments, the intermediate probe comprises (i) a recognition sequence complementary to the identifier sequence in the RCP and (ii) an overhang sequence. In some embodiments, the fluorescently labeled probe comprises (i) a sequence complementary to the overhang sequence and (ii) a fluorescent label. In some embodiments, the method provided herein further comprises using the optics module to detect first signals (or absence thereof) associated with the fluorescent labels of the first plurality of probe pairs at multiple locations on the solid support. In some embodiments, the first signal or absence thereof detected at a particular location corresponds to a first signal code in the signal code sequence assigned to the identifier sequence in the RCP at the particular location.

In some embodiments, the method provided herein further comprises: in a second cycle, using the fluidics module to deliver to the solid support a second plurality of intermediate probe/fluorescently labeled probe pairs. In some embodiments, the intermediate probe and the fluorescently labeled probe in each pair form a complex comprising the intermediate probe hybridized to an RCP of the plurality of RCPs and the fluorescently labeled probe hybridized to the intermediate probe. In some embodiments, the intermediate probe comprises (i) a recognition sequence complementary to the identifier sequence in the RCP and (ii) an overhang sequence. In some embodiments, the fluorescently labeled probe comprises (i) a sequence complementary to the overhang sequence and (ii) a fluorescent label. In some embodiments, the method provided herein further comprises using the optics module to detect second signals (or absence thereof) associated with the fluorescent labels of the second plurality of probe pairs at multiple locations on the solid support. In some embodiments, the second signal or absence thereof detected at a particular location corresponds to a second signal code in the signal code sequence assigned to the identifier sequence in the RCP at the particular location, thereby generating a signal code sequence comprising at least the first signal code and the second signal code at each of the multiple locations. In some embodiments, the signal code sequence comprising the first signal code, the second signal code, a third signal code corresponding to a third cycle, and a fourth signal code corresponding to a fourth cycle. Optionally, in some embodiments, the signal code sequence comprises a dark signal code corresponding to the absence of signal in the corresponding cycle.

In some embodiments, the method provided herein further comprises using the system controller to compare the generated signal code sequences for the RCPs at the multiple locations to those from the codebook, thereby decoding the identifier sequences in the RCPs. In some embodiments, the method provided herein further comprises qualifying the instrument for detecting analytes in a biological sample based on the decoding in the decoding step (i.e., the step of using the system controller to compare the generated signal code sequences for the RCPs at the multiple locations to those from the codebook, thereby decoding the identifier sequences in the RCPs). In some embodiments, the method provided herein comprises using the qualified instrument to decode analytes in situ in the biological sample. In some embodiments, when at least or about 90% of the different identifier sequences in the RCPs are decoded in the decoding step (i.e., in the step of using the system controller to compare the generated signal code sequences to those from the codebook, thereby decoding the identifier sequences in the RCPs), the instrument is qualified as suitable for decoding analytes in situ in the biological sample.

In some embodiments, the method provided herein further comprises using the system controller to compare the generated signal code sequences for the RCPs at the multiple locations to those from the codebook, thereby decoding the identifier sequences in the RCPs. In some embodiments, the method provided herein further comprises qualifying the instrument for detecting analytes in a biological sample based on the decoding in the decoding step (i.e., the step of using the system controller to compare the generated signal code sequences for the RCPs at the multiple locations to those from the codebook, thereby decoding the identifier sequences in the RCPs). In some embodiments, the method provided herein comprises using the qualified instrument to decode analytes in situ in the biological sample. In some embodiments, when at least or about 90% of the different identifier sequences in the RCPs are decoded in the decoding step (i.e., in the step of using the system controller to compare the generated signal code sequences to those from the codebook, thereby decoding the identifier sequences in the RCPs), the instrument is qualified as suitable for decoding analytes in situ in the biological sample.

In some embodiments, in the first cycle, a first pool of intermediate probes and a universal pool of fluorescently labeled probes are delivered to the solid support. In some embodiments, each intermediate probe in the first pool of intermediate probes comprises (i) a recognition sequence complementary to one of the different identifier sequences in the RCPs, and (ii) a hybridization sequence complementary to a fluorescently labeled probe of the universal pool. In some embodiments, in the second cycle, a second pool of intermediate probes and the universal pool of fluorescently labeled probes are delivered to the solid support. In some embodiments, each intermediate probe in the second pool of intermediate probes comprises (i) a recognition sequence complementary to one of the different identifier sequences in the RCPs, and (ii) a hybridization sequence complementary to a fluorescently labeled probe of the universal pool. In some embodiments, the number of different identifier sequences in the RCPs is at least 9 and the number of fluorescently labeled probes of different sequences in the universal pool is 4. In some embodiments, each fluorescently labeled probe of a different sequence in the universal pool is labeled with a fluorophore of a different color.

In some embodiments, herein is provided a method for producing a slide, comprising separately generating rolling circle amplification products (RCPs) of each circular template of a plurality of different circular templates. In some embodiments, the RCPs of each circular template comprise multiple copies of a different identifier sequence which is assigned a different signal code sequence from a codebook. In some embodiments, the method for producing a slide provided herein further comprises pooling RCPs of the plurality of different circular templates. In some embodiments, the method for producing a slide provided herein further comprises disposing the pooled RCPs on a solid support, thereby producing the slide comprising the solid support and the pooled RCPs thereon. In some embodiments, the pooled RCPs are not in a cell or tissue. Optionally, in some embodiments, the RCPs are pooled in solution or in lyophilized form. In some embodiments, the pooled RCPs are disposed in two, three, four, or more discrete regions on the solid support. In some embodiments, the solid support comprises one or more fiducial markers. In some embodiments, the method provided herein further comprises disposing a plurality of particles on the solid support prior to, concurrently with, or after disposing the pooled RCPs on the solid support. In some embodiments, the particles are latex beads coupled to DAPI. In some embodiments, the ratio between the number of the particles and the number of the RCPs is between about 1:500 and about 1:1,500. Optionally, in some embodiments, the ratio between the number of the particles and the number of the RCPs is about 1:1,000.

In some embodiments, the RCPs of each circular template are generated outside of a cell or tissue sample, generated in solution, and/or generated in vitro. In some embodiments, the provided method comprises providing or generating each circular template separately in a solution. In some embodiments, the circular template is generated by hybridizing a circularizable probe or probe set to a splint oligonucleotide, and circularizing the circularizable probe or probe set using the splint oligonucleotide as a template. In some embodiments, the circularizing comprises ligation templated on the splint oligonucleotide, with or without gap filling prior to the ligation. In some embodiments, the solution comprising the circular template is diluted prior to rolling circle amplification of the circular template.

In some embodiments, the RCPs of each circular template comprise modified nucleic acid residues. In some embodiments, amine-modified nucleic acid residues are incorporated during rolling circle amplification. In some embodiments, the solid support comprises functional groups configured to bind to or react with the modified nucleic acid residues, thereby immobilizing the RCPs on the solid support. In some embodiments, the functional groups comprise an N-hydroxysuccinimide (NHS) moiety configured to react with amines in the RCPs and/or in the particles.

### EXAMPLES

The following examples are included for illustrative purposes only and are not intended to limit the scope of the present disclosure.

### Example 1: Instrument Performance Test Utilizing In Vitro Synthesized RCPs on Slide

RCPs for decoding nine genes according to a codebook were synthesized in vitro. The RCPs and beads labeled with a blue fluorescent dye were deposited on a solid support to generate a quality control (QC) slide. The QC slide was mounted on an instrument to be assessed by an instrument performance test (IPT), and the instrument was run to deliver intermediate probes and detectably labeled probes to the QC slide, image the QC slide in each probe hybridization cycle, and detect and process signals from locations of RCPs detected in the sequential cycles. Performance metrics of the IPT were calculated and used to assess instrument performance.

For in vitro RCP synthesis, RCPs for each different gene were synthesized in separate tubes. In each tube, a padlock probe and its corresponding splint oligonucleotide were added to a hybridization buffer and a probe dilution buffer and incubated. A ligase and a ligation buffer were added for ligation of the padlock probe in each padlock probe/splint oligonucleotide pair, and the reaction mixture was incubated. After dilution, RCA was carried out in each tube separately using a reaction mixture containing a polymerase, RCA reaction buffer, and dNTPs with aminoallyl-dUTP spiked in. The reaction mixture was incubated at room temperature before incubation on ice. The RCPs from the nine tubes were then pooled together.

For RCP printing and slide preparation, a slide was removed from a Mylar bag and a cassette was added to the slide. The pooled RCA reactions were vortexed to mix and combined with a printing buffer. Beads labeled with the blue fluorescent dye were then added to the printing buffer mixture and mixed. The printing buffer mixture containing RCPs and beads was then spotted onto each of the four corners of the slide next to fiduciary markers. The slide was then incubated at room temperature until the spots were completely dry. The cassette was then placed in a humidity chamber. The slide was then removed and washed with a stripping buffer to remove nonspecific binding. After washing, the slide was dried and the cassette was removed. The QC slide can be stored at −20° C. until use.

For probe hybridization, a cassette was applied to the QC slide. For a cycle of detection, a pool of intermediate probes targeting RCPs of different genes were added and incubated, and fluorescently detected oligonucleotides were added to detect the intermediate probes. Different pools of intermediate probes were cycled on the QC slide and a universal pool of fluorescently detected oligonucleotides were used to detect the intermediate probes in each cycle. Imaging buffer was added to the QC slide for RCP visualization and decoding.

Through the sequential hybridization and detection cycles, fluorescent signals from the RCPs were detected and recorded at locations in various regions of interest (ROIs) in the QC slide. The order of signals (or absence thereof) at a given location through the multiple cycles provided a signal code sequence for the RCP at the location, and the signal code sequence was compared to those in the codebook to identify a corresponding barcode sequence in the RCP and the gene associated therewith.

Beads labeled with a blue fluorescent dye were imaged in the first cycle and used to mimic nuclei count and segmentation of cells. RCPs were decoded as transcripts and gene counts were compared to that from a dummy sample which served as a negative control. These results indicated that the IPT generated biological sample-like decoding data.

Pass/fail metrics for qualifying an instrument were developed using in vitro synthesized RCPs on QC slides. Examples of metrics are listed in Table 1 below. For instance, an instrument passes the IPT and can be used for in situ analysis of a tissue sample if all the targets are met across all the eight ROIs of the IPT. For instance, an instrument does not passes the IPT and cannot be used for in situ analysis of a tissue sample if any FOV shows zero object decoded to genes with Q-score >=20.

TABLE 1
Examples of IPT Pass/Fail Metrics

| | Target |
|---|---|
| Metric Names | Values |
| Fraction of objects decoded to gene with Q-score >=20 | >=40% |
| Gene density with Q-score >=20 (in obj_per_μm \hat{ }2) | >=0.0003 |
| Thickness of decoded objects Q-score >=20 (in μm) | <=0.8 |
| Maximum decoding false positive rate | <=15% |
| Maximum decoding false negative rate | <=90% |
| Minimal objects decoded to gene with Q-score >=20 per | >=1 |
| FOV | |
| Quartile coefficient of dispersion of Q20 genes per FOV | <=60% |
| Number of cells detected | >=500 |

Together, these data demonstrate robust instrument performance tests using in vitro synthesized RCPs on QC slides. Pass/fail metrics can be used to assess an instrument as pass (e.g., satisfactory performance) or fail (e.g., unsatisfactory performance). The instrument performance tests can be employed by a user to confidently assess performance of an instrument and the instrument workflow before in situ analyte detection assays on cell or tissue samples using the instrument.

### Example 2: In Vitro Synthesis of RCPs Attached to Gel Beads

RCPs were synthesized in vitro using particles (e.g., gel beads) generated by a membrane emulsification process, and the particles were functionalized with an oligonucleotide comprising an acrydite moiety. The generated particles comprise a plurality of oligonucleotides comprising a primer sequence (as shown in FIG. 4). The gel beads with the functionalized oligonucleotides were incubated with circularized probes in solution to generate RCPs. The synthesized RCPs on gel beads were then deposited on a solid support to generate a quality control (QC) slide. As described in Example 1, the generated QC slide was then used to assess instrument performance.

To generate the beads, a monomer solution with acrylamide and acrydite-modified oligonucleotides was combined with sodium formate and ammonium persulfate solutions. Particles were generated by priming a porous membrane with oil, loading the aqueous solution and passing through the membrane under constant stirring, creating emulsions in oil. After the entire volume of aqueous solution was passed through the membrane, TEMED was added and mixed into the emulsion layer to initiate polymerization. After polymerization was completed, the emulsion layer was removed from oil and the emulsions were broken. The residual oil was then removed through a series of wash steps, leaving the beads suspended in buffer solution. The membrane pore size, monomer solution formation, and degree of crosslinking were adjusted to achieve the desired size bead of about 10 μm in diameter. A fraction of the generated beads was visualized and quantified to measure and determine the bead concentration and size. From the generation of the gel beads, 10 μm diameter beads were generated which allows for higher RCP density and the RCP shape was closer to that observed within a tissue sample.

During gel bead generation, acrydite oligonucleotides (e.g., anchor oligonucleotides) were incorporated into the functionalized gel beads. The anchor oligonucleotide comprises a sequence for hybridizing to a mixture of different circularized probes associated with a panel of reference genes. For in vitro RCP synthesis, RCA was carried out on the gel beads using a reaction mixture containing a polymerase, RCA reaction buffer, and dNTPs. The generated gel beads with RCPs were then spotted onto each of the four corners of a slide with functional groups. In some cases, a mono layer of gel beads adhered to the slide surface is desired. The slide was then incubated at room temperature until the spots were completely dry. The QC slide can be stored at −20° C. until use. In some cases, the anchor oligonucleotide or the RCP may be used to hybridize a detectably labeled oligonucleotide (e.g., to couple with a blue dye).

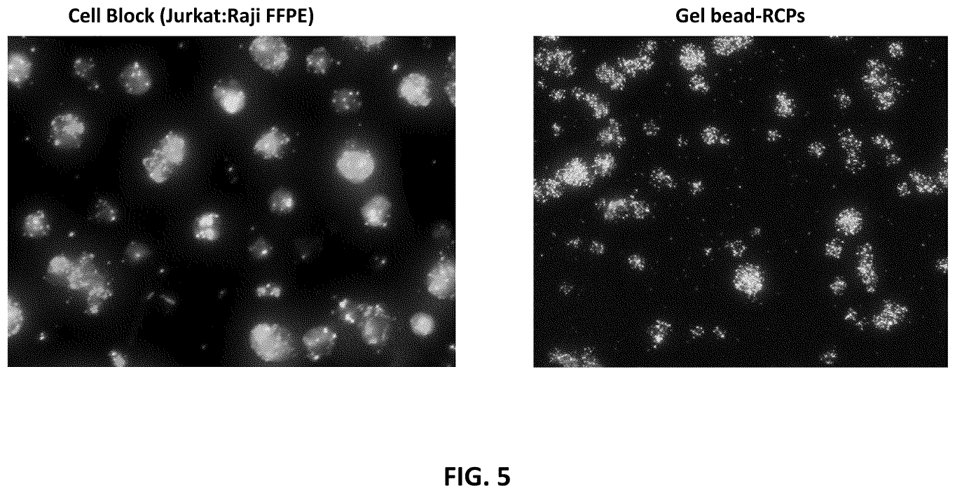

Probe hybridization and detection in sequential cycles was performed, and fluorescent signals from the RCPs were detected and recorded at locations in various regions of interest (ROIs) in the QC slide substantially as described in Example 1. As shown in FIG. 5, RCPs generated and deposited onto the slide with the gel beads showed comparable signals as signals observed from RCPs generated in a sample from a cell block containing two different cell types. In some cases, QC slides with RCPs generated on gel beads allow for higher density which is useful for assessing instrument performance for assays. In some aspects, the diameter of the bead provides a density of RCPs that is detected by imaging a depth (e.g., z-stack) and mimics a cell or tissue sample.

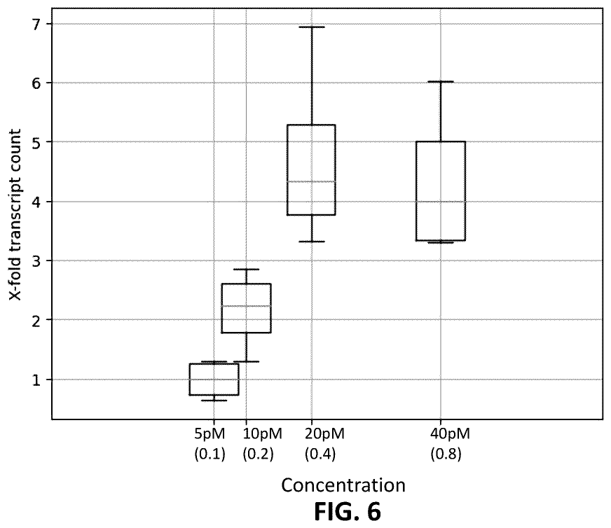

In a separate experiment, gel beads were generated as described, various concentrations (5 pM, 10 pM, 20 pM, and 40 pM) of circularized probes associated with a panel of reference genes were used to generate RCPs, and the gel beads were spotted on slides. Probe hybridization and detection in sequential cycles was performed, and fluorescent signals from the RCPs were detected and recorded at locations in various regions of interest (ROIs) in the QC slide. As shown in FIG. 6, a trend of increasing detected RCP count (“transcript count”) was observed with increased probe concentration.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-153. (canceled)

154. A kit, comprising:
a plurality of rolling circle amplification products (RCPs),
a plurality of beads, and
a solid support comprising functional groups for immobilizing the plurality of RCPs and/or the plurality of beads on the solid support.

155. The kit of claim 154, wherein:
the plurality of RCPs are immobilized on the solid support, and/or
the plurality of beads are immobilized on the solid support.

156. The kit of claim 154, wherein the plurality of beads comprise beads coupled to detectable labels.

157. The kit of claim 156, wherein the diameters of the beads are between about 0.5 μm and about 3 μm.

158. The kit of claim 156, wherein the detectable labels have an excitation wavelength between about 300 nm and about 400 nm.

159. The kit of claim 156, wherein the detectable labels have an emission wavelength between about 400 nm and about 600 nm.

160. The kit of claim 156, wherein the detectable labels are non-autofluorescent or substantially nonfluorescent under excitation wavelengths between 490 nm and 650 nm

161. The kit of claim 157, wherein the beads are hydrogel beads or latex beads.

162. The kit of claim 154, wherein the RCPs are generated in solution outside a cell or tissue sample.

163. The kit of claim 154, wherein each RCP comprises multiple copies of an identifier sequence, and wherein the kit additionally comprises detectably labeled probes configured to hybridize to the identifier sequences in the RCPs, or comprises intermediate probes configured to hybridize to the identifier sequences in the RCPs and detectably labeled probes configured to hybridize to at least some of the intermediate probes.

164. The method of claim 165, wherein the identifier sequences in the RCPs comprise sequences of a set of reference genes or complements thereof, and/or wherein each RCP is associated with an assigned signal code sequence from a codebook.

165. The kit of claim 164, wherein the solid support comprises a planar glass or substrate.

166. The kit of claim 165, wherein the plurality of RCPs and/or the plurality of beads are immobilized in two or more discrete regions on the solid support.

167. The kit of claim 166, wherein the plurality of RCPs and/or the plurality of beads comprise a functional group configured to react with the functional groups of the solid support.

168. The kit of claim 167, wherein a plurality of amine-modified nucleic acid residues is incorporated into the plurality of RCPs during rolling circle amplification.

169. A method for qualifying a system comprising an instrument, comprising:
a) placing a solid support on the instrument,
wherein the system comprises:
the instrument,
reagents comprising fluorescently labeled probes,
a fluidics module,
an optics module, and
a system controller, and
wherein the solid support comprises rolling circle amplification products (RCPs) deposited thereon, each RCP comprising multiple copies of an identifier sequence having an assigned signal code sequence from a codebook, and wherein the solid support comprises a plurality of beads deposited thereon;
b) using the fluidics module to deliver, in sequential cycles, the fluorescently labeled probes to the solid support;
c) using the optics module to detect, in the sequential cycles, signals associated with the fluorescently labeled probes directly or indirectly bound to the identifier sequences in the RCPs, thereby generating signal code sequences for the RCPs;
d) using the system controller to compare the generated signal code sequences to those from the codebook, thereby decoding the identifier sequences in the RCPs; and
e) qualifying the system, wherein the system is suitable for detecting analytes in a biological sample when the decoding in d) meets one or more pre-defined criteria.

170. The method of claim 169, wherein the solid support: does not comprise a cell or tissue sample thereon.

171. The method of claim 169, wherein the plurality of beads is coupled to a blue fluorescent dye.

172. The method of claim 169, wherein the plurality of beads comprise functionalized gel beads or latex beads comprising acrydite moieties.

173. The method of claim 169, wherein the one or more pre-defined criteria comprise any one or more of:
at least or about 35% of detected RCPs are decoded;
the number of detected RCPs per μm² with a quality score of at least 20 is greater than or about 0.0001;
the thickness of detected RCPs with a quality score of at least 20 is less than or about 1 μm;
the maximum decoding false positive rate is less than or about 20%;
the maximum decoding false negative rate is less than or about 80%;
the minimal number of detected RCPs that are decoded with a quality score of at least 20 per field of view (POV) is greater than 0;
the quartile coefficient of dispersion of decoded different identifier sequences with a quality score of at least 20 per field of view (POV) is less than or about 65%; and
the number of particles detected is at least or about 400.
